(19) United States
(12) Patent Application Publication
(10) Pub. No.: US 2014/0314784 A1
(43) Pub. Date: Oct. 23, 2014

(54) ANTI-CXCR4 ANTIBODIES AND METHODS OF USE

(71) Applicants: Vahe Bedian, Waltham, MA (US); Youzhen Wang, Newton, MA (US); Ian Foltz, Burnaby (CA); Palaniswami Rathanaswami, Vancouver (CA); Jaspal Singh Kang, Surrey (CA); Adeela Kamal, Gaithersburg, MD (US); Philipp Steiner, Gaithersburg, MD (US)

(72) Inventors: Vahe Bedian, Waltham, MA (US); Youzhen Wang, Newton, MA (US); Ian Foltz, Burnaby (CA); Palaniswami Rathanaswami, Vancouver (CA); Jaspal Singh Kang, Surrey (CA); Adeela Kamal, Gaithersburg, MD (US); Philipp Steiner, Gaithersburg, MD (US)

(73) Assignee: Medlmmune Limited, Cambridge (GB)

(21) Appl. No.: 14/232,751

(22) PCT Filed: Jul. 19, 2012
(86) PCT No.: PCT/US2012/047370

§ 371 Date: Apr. 23, 2014

Related U.S. Application Data
(60) Provisional application No. 61/509,674, filed on Jul. 20, 2011.

(51) Int. Cl. C07K 16/28; A61K 45/06; A61K 39/395

(52) U.S. Cl. CPC C07K 16/2866; A61K 39/3955; A61K 45/06
USPC 424/144.1; 530/388.22; 536/23.53

(57) ABSTRACT

The disclosure relates to antibodies or antigen binding fragments that specifically bind to CXCR4 and inhibit the biological activity of CXCR4 and uses of such agents. More specifically the disclosure relates to fully human antibodies or antigen binding fragments directed to CXCR4 that specifically bind to CXCR4 and uses of these antibodies. Aspects of the disclosure also relate to hybridomas or other cell lines expressing such antibodies. The disclosed antibodies (including antigen binding fragments) are useful as diagnostics and for the treatment of diseases associated with the activity and/or expression of CXCR4.

### CLAIM OF PRIORITY

This application claims the benefit of prior U.S. Provisional Application No. 61/509,674, filed on Jul. 20, 2011, which is incorporated by reference in its entirety.

### FIELD OF THE DISCLOSURE

The present disclosure relates to anti-CXCR4 (chemokine C-X-C motif receptor 4) antibodies or antigen binding fragments and methods of using them.

### BACKGROUND OF THE DISCLOSURE

CXCR4 is a G-protein coupled receptor that mediates the activity of CXC chemokines. To date, its only identified cognate ligand is CXCL12, also known as Stromal Cell-Derived Factor-1 (SDF-1). CXCR4 plays an important role in mammalian development, mediating the migration and motility of tissue and hematopoietic stem and progenitor cells. Both CXCR4 and SDF-1 knock-out mice show embryonic lethality with essentially identical phenotypes involving tissue and vascular malformations, supporting the hypothesis that CXCR4 is the key receptor for the activity of SDF-1 (CXCR7 is a second known receptor of SDF-1). CXCR4 continues to be broadly expressed in the adult, with high levels detected on bone marrow stem and progenitor cells, various circulating lymphocytes (B-cells, activated T-cells), as well as endothelial precursor cells, and tissue macrophages and fibroblasts.

CXCR4 is likely to play a pleiotropic role in human cancer. Its expression is upregulated in many tumor types, including cancers of the breast, lung, colon, pancreas, brain, prostate, ovary, as well as hematopoietic cancers. Some literature reports suggest that SDF-1 may act through CXCR4 as a growth and/or survival factor for some tumors. In models of metastatic cancer, CXCR4 positive tumors were shown to metastasize to distant sites, and this activity was inhibited by agents that silence the CXCR4 gene or antibodies that block CXCR4 or SDF-1. Consistent with this view, many common sites of metastasis in human cancer, such as bone marrow, lung, lymph node, and liver, express high levels of SDF-1. CXCR4 is expressed on stem cell-like or tumor initiating subpopulations of many tumors, and may mediate the ability of these cells to support the recurrence and metastatic spread of cancers. Furthermore, CXCR4 is expressed on endothelial precursor cells (EPCs), and its activity is required for incorporation of EPCs into functional vessels during angiogenesis. This may make a significant contribution to the vascularization and survival of tumors. CXCR4 signaling can also lead to induction of pro-angiogenic cytokines (e.g. VEGF), as well as integrins, adhesion molecules and matrix degrading enzymes that may mediate invasion by tumor cells. Lastly, CXCR4 expression is detected on tumor infiltrating lymphocytes and fibroblasts, as well as tumor associated macrophages. These cells tend to suppress immune recognition and attack on the tumor, and remodel the tumor microenvironment to encourage tumor growth and metastasis.

The multiple roles of CXCR4 in tumor growth, vascularization, and metastasis, and its broad expression in many common tumor types, make this receptor an attractive target for therapeutic intervention using inhibitory agents. While both peptide and small molecule inhibitors of CXCR4 have been identified and entered into the clinic, their utility has been limited by pharmacokinetic properties and toxicology. At present, the bicyclam AMD3100 is approved for mobilization of hematopoietic precursors from the bone marrow for autologous stem cell transplantation. An agent that is selective, has a long half-life, and is safe would be a desirable agent for use in the treatment of cancers, as well as for the mobilization of stem cells.

### SUMMARY OF THE DISCLOSURE

The present disclosure relates to antibodies or antigen binding fragments that specifically bind to CXCR4 and inhibit the biological activity of CXCR4. Such antibodies or antigen binding fragments, also referred to as anti-CXCR4 antibodies or antigen binding fragments may specifically bind to CXCR4 and thereby inhibit CXCR4 receptor activity. Targeted binding agents may also specifically bind to CXCR4 and thereby inhibit ligand, e.g., SDF-1, induced cell proliferation (for example tumour cell proliferation), ligand, e.g., SDF-1, induced cell survival (for example tumour cell survival) or ligand, e.g., SDF-1, induced cell motility (e.g. stem cell mobilization, tumor cell metastasis, or endothelial precursor cell motility).

In certain embodiments of the disclosure, antibodies or antigen binding fragments specifically bind to CXCR4 and thereby inhibit binding of SDF-1- to CXCR4. In further embodiments, antibodies or antigen binding fragments specifically bind to CXCR4 and thereby inhibit chemotaxis of cells, such as tumor cells, endothelial precursor cells, lymphocytes, monocytes, macrophages, and fibroblasts, expressing CXCR4. In other embodiments, antibodies or antigen binding fragments specifically bind to CXCR4 and thereby inhibit induction of cellular mediators and cytokines such as angiogenic factors, immune modulatory cytokines, integrins, and matrix metalloproteases. Numerous examples of such anti-CXCR4 antibodies are provided herein.

The disclosure contemplates various combinations of any of the features and uses described herein. For example, any of the anti-CXCR4 antibodies or antigen binding fragments of the disclosure, may be used in any of the diagnostic or therapeutic methods described herein and/or may be described based on any one or more (2, 3, 4, 5, 6, 7, 8, 9, etc.) of the functional or structural features described herein. Moreover, any such antibodies or antigen binding fragments may be modified, as described herein, and any of these antibodies may be described using structural or functional characteristics.

### BRIEF DESCRIPTION OF THE FIGURES

FIG. 1 shows line graphs representing dose-response curves for various antibodies in inhibition of Jurkat cell chemotaxis. FIG. 1A provides a graph for the antibody referred to herein as 6C7. FIG. 1B provides a graph for the antibody referred to herein as 4C1. FIG. 1C provides a graph for the antibody referred to herein as 7C8. FIG. 1D provides a graph for the antibody referred to herein as 2A4.

FIGS. 2A and 2B depicts inhibitory activity of antibody 6C7 in U937 (2A) and HSC-F (2B) chemotaxis assays.

FIG. 3 shows the results demonstrating blocking of labeled SDF-1 binding to Namalwa cells by antibody 6C7.

FIG. 4 provides a bar chart (FIG. 4A) and line graph (FIG. 4B) showing inhibition of SDF-1 induced phospho-Erk signal in Jurkat cells.

FIG. 5 provides a bar chart showing inhibition of SDF-1 induced phospho-AKT signal in HSC-F cells.

FIG. 6 provides line graphs depicting Kinexa analysis of on-cell affinity of antibodies to human CXCR4 on Namalwa cells. FIG. 6A depicts the analysis using the 6C7 monoclonal antibody described herein. FIG. 6B depicts analysis using a reference antibody.

FIG. 7 provides line graphs depicting Kinexa analysis of on-cell affinity of antibodies to cynomolgus CXCR4 on HSC-F cells. FIG. 7A depicts the analysis using the 6C7 monoclonal antibody described herein. FIG. 7B depicts analysis using a reference antibody

FIG. 8 is a bar chart showing induction of apoptosis by various antibodies in Ramos cells.

FIG. 9 depicts the results of experiments evaluating the antiangiogenic efficacy of cxcr4 antibodies in a spheroid-based in vivo angiogenesis assay.

FIG. 10 depicts the results of experiments evaluating the activity of an anti-CXCR4 antibody of the disclosure in a model of ovarian cancer.

FIG. 11 depicts the results of experiments evaluating the effects of an anti-CXCR4 antibody of the disclosure on wound healing in a scratch-test wound healing model.

FIG. 12 depicts the results of experiments evaluating the activity of an anti-CXCR4 antibody of the disclosure in a multiple myeloma model.

FIG. 13 depicts the results of experiments evaluating the activity of an anti-CXCR4 antibody of the disclosure in a Burkitt's lymphoma model.

FIG. 14A depicts the results of experiments evaluating the effects of an anti-CXCR4 antibody of the disclosure on ovarian cancer disseminated intravenous model to lungs. FIG. 14B depicts a scatter plot of individual mice lungs ex vivo imaged 33 days after treatment with anti-CXCR4 antibody of the disclosure. FIG. 14C depicts images of lungs of mice treated with control antibody and with anti-CXCR4 antibody of the disclosure.

FIG. 15A depicts the results of experiments evaluating the effects of an anti-CXCR4 antibody of the disclosure in a chronic lymphocytic leukemia (CLL) model. FIG. 15B depicts the results of experiments evaluating the effects of an anti-CXCR4 antibody of the disclosure alone and in combination with Rituxan in a second chronic lymphocytic leukemia (CLL) model.

FIG. 16 shows the results of an epitope mapping experiment. Domain swaps of human CXCR4 with mouse CXCR4 showed that antibody 6C7 binds the second loop of CXCR4. The second loop in human CXCR4 is shorter from mouse CXCR4 by 5 amino acids. Also, the second loop has 7 individual residue differences. The first of these single amino acid differences results in loss of an N-glycosylation consensus sequence that is present in human but not in mouse CXCR4.

### DETAILED DESCRIPTION

### (i) Terminology

Before describing the present disclosure in additional detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. Further, unless otherwise required by context, singular terms shall include pluralities and plural terms shall include the singular. Generally, nomenclatures utilised in connection with, and techniques of, cell and tissue culture, molecular biology, and protein and oligo- or polynucleotide chemistry and hybridisation described herein are those well known and commonly used in the art.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)), which is incorporated herein by reference.

The term “CXCR4” refers to the human CD 184, CD 184 antigen, C-X-C chemokine receptor type 4, CXCR-4, CXCL-12, CXC-R4, D2S201E, FB22, fusin, Fusin, HM89, HSY3RR, LAP3, LCR1, LESTR, Leukocyte-derived seven transmembrane domain receptor, NPY3R, NPYR, NPYRL, NPYY3R, SDF-1 receptor, or Stromal cell-derived factor 1 receptor.

The term “neutralising” or “inhibits” when referring to an antibody or antigen binding fragment of the disclosure, relates to the ability of said antibody or antigen binding fragment to eliminate, reduce, or significantly reduce, the activity of a target antigen, such as CXCR4, for example, by reducing the biological activity of the target antigen in comparison with the biological activity in the absence of an antibody or antigen binding fragment of the disclosure by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, where the reduction of CXCR4 biological activity can be measured, for example, using any one of the in vitro or in vivo assays as described further below and in the Examples, or known to one of ordinary skill in the art.

Accordingly, an “inhibiting” or “neutralising” anti-CXCR4 antibody or antigen binding fragment of the disclosure is capable of eliminating or significantly reducing the biological activity of CXCR4. The biological activity of CXCR4 corresponds to, for example, any one of a number of activities including tumor growth and/or survival, SDF-1 induced cellular metastasis, phosphorylation of phosphor-MAP kinase including Erk1 and Erk2 and/or AKT kinase, SDF-1 induced MAP kinase phosphorylation, cell proliferation (e.g., in response to SDF-1 ligand), cell adhesion or invasion. A neutralising, antagonising or inhibiting antibody that specifically binds CXCR4 may, for example, act by blocking the binding of SDF-1 to the CXCR4 receptor. Ideally, a neutralising antibody against CXCR4 inhibits cell proliferation, cell adhesion and invasion.

The term “selectively hybridise” referred to herein means to detectably and specifically bind. Polynucleotides, oligonucleotides and fragments thereof selectively hybridise to nucleic acid strands under hybridisation and wash conditions that minimise appreciable amounts of detectable binding to nonspecific nucleic acids. High stringency conditions can be used to achieve selective hybridisation conditions as known in the art and discussed herein. Generally, the nucleic acid sequence homology between the polynucleotides, oligonucleotides, or antigen binding fragments and a nucleic acid sequence of interest will be at least 80%, and more typically with preferably increasing homologies of at least 85%, 90%, 95%, 99%, and 100%.

Stringent hybridization conditions include, but are not limited to, hybridization to filter-bound DNA in 6× sodium chloride/sodium citrate (SSC) (0.9 M NaCl/90 mM NaCitrate, pH 7.0) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

In general, cysteine residues in proteins are either engaged in cysteine-cysteine disulfide bonds or sterically protected from the disulfide bond formation when they are a part of folded protein region. Disulfide bond formation in proteins is a complex process, which is determined by the redox potential of the environment and specialized thiol-disulfide exchanging enzymes (Creighton, Methods Enzymol. 107, 305-329, 1984; Houee-Levin, Methods Enzymol. 353, 35-44, 2002). When a cysteine residue does not have a pair in protein structure and is not sterically protected by folding, it can form a disulfide bond with a free cysteine from solution in a process known as disulfide shuffling. In another process known as disulfide scrambling, free cysteines may also interfere with naturally occurring disulfide bonds (such as those present in antibody structures) and lead to low binding, low biological activity and/or low stability.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterises the parent sequence). Examples of art-recognised polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference. Additionally, such methods may be used to make amino acid substitutions or deletions of one or more variable region cysteine residues participating in an intrachain disulfide bond to generate antibody molecules lacking one or more intrachain disulfide bonds.

The term “CDR region” or “CDR” is intended to indicate the hypervariable regions of the heavy and light chains of an antibody which confer the antigen-binding specificity to the antibody. CDRs may be defined according to the Kabat system (Kabat, E. A. et al. (1991) Sequences of Proteins of Immunological Interest, 5th Edition. US Department of Health and Human Services, Public Service, NIH, Washington), and later editions. An antibody typically contains 3 heavy chain CDRs and 3 light chain CDRs. The term CDR or CDRs is used here in order to indicate, according to the case, one of these regions or several, or even the whole, of these regions which contain the majority of the amino acid residues responsible for the binding by affinity of the antibody for the antigen or the epitope which it recognises.

The third CDR of the heavy chain (HCDR3) has a greater size variability (greater diversity essentially due to the mechanisms of arrangement of the genes which give rise to it). It may be as short as 2 amino acids although the longest size known is 26. CDR length may also vary according to the length that can be accommodated by the particular underlying framework. Functionally, HCDR3 plays a role in part in the determination of the specificity of the antibody (Segal et al., PNAS, 71:4298-4302, 1974, Amit et al., Science, 233:747-753, 1986, Chothia et al., J. Mol. Biol., 196:901-917, 1987, Chothia et al., Nature, 342:877-883, 1989, Caton et al., J. Immunol., 144:1965-1968, 1990, Sharon et al., PNAS, 87:4814-4817, 1990, Sharon et al., J. Immunol., 144:4863-4869, 1990, Kabat et al., J. Immunol., 147:1709-1719, 1991).

The term a “set of CDRs” referred to herein comprises CDR1, CDR2 and CDR3. Thus, a set of HCDRs refers to HCDR1, HCDR2 and HCDR3, and a set of LCDRs refers to LCDR1, LCDR2 and LCDR3.

Variants of the VH and VL domains and CDRs of the present disclosure, including those for which amino acid sequences are set out herein, and which can be employed in targeting binding agents and antibodies for CXCR4 can be obtained by means of methods of sequence alteration or mutation and screening for antigen targeting with desired characteristics. Examples of desired characteristics include but are not limited to: increased binding affinity for antigen relative to known antibodies which are specific for the antigen; increased neutralisation of an antigen activity relative to known antibodies which are specific for the antigen if the activity is known; specified competitive ability with a known antibody or ligand to the antigen at a specific molar ratio; ability to immunoprecipitate ligand-receptor complex; ability to bind to a specified epitope; linear epitope, e.g. peptide sequence identified using peptide-binding scan, e.g. using peptides screened in linear and/or constrained conformation; conformational epitope, formed by non-continuous residues; ability to modulate a new biological activity of CXCR4, or downstream molecule; ability to bind and/or neutralise CXCR4 and/or for any other desired property. The techniques required to make substitutions within amino acid sequences of CDRs, antibody VH or VL domains and antigen binding sites are available in the art. Variants of antibody molecules disclosed herein may be produced and used in the present disclosure. Following the lead of computational chemistry in applying multivariate data analysis techniques to the structure/property-activity relationships (Wold, et al. Multivariate data analysis in chemistry. Chemometrics—Mathematics and Statistics in Chemistry (Ed.: B. Kowalski), D. Reidel Publishing Company, Dordrecht, Holland, 1984) quantitative activity-property relationships of antibodies can be derived using well-known mathematical techniques, such as statistical regression, pattern recognition and classification (Norman et al. Applied Regression Analysis. Wiley-Interscience; 3rd edition (April 1998); Kandel, Abraham & Backer, Eric. Computer-Assisted Reasoning in Cluster Analysis. Prentice Hall PTR, (May 11, 1995); Krzanowski, Wojtek. Principles of Multivariate Analysis: A User's Perspective (Oxford Statistical Science Series, No 22 (Paper)). Oxford University Press; (December 2000); Witten, Ian H. & Frank, Eibe. Data Mining: Practical Machine Learning Tools and Techniques with Java Implementations. Morgan Kaufmann; (Oct. 11, 1999); Denison David G. T. (Editor), Christopher C. Holmes, Bani K. Mallick, Adrian F. M. Smith. Bayesian Methods for Nonlinear Classification and Regression (Wiley Series in Probability and Statistics). John Wiley & Sons; (July 2002); Ghose, Amp K. & Viswanadhan, Vellarkad N. Combinatorial Library Design and Evaluation Principles, Software, Tools, and Applications in Drug Discovery). In some cases the properties of antibodies can be derived from empirical and theoretical models (for example, analysis of likely contact residues or calculated physicochemical property) of antibody sequence, functional and three-dimensional structures and these properties can be considered singly and in combination.

This study of sequence-structure relationship can be used for prediction of those residues in an antibody of known sequence, but of an unknown three-dimensional structure, which are important in maintaining the three-dimensional structure of its CDR loops and hence maintain binding specificity. These predictions can be backed up by comparison of the predictions to the output from lead optimisation experiments. In a structural approach, a model can be created of the antibody molecule using any freely available or commercial package, such as WAM. A protein visualisation and analysis software package, such as Insight II (Accelrys, Inc.) or Deep View may then be used to evaluate possible substitutions at each position in the CDR. This information may then be used to make substitutions likely to have a minimal or beneficial effect on activity or confer other desirable properties.

As used herein “antibody” and “antibodies” (immunoglobulins) may be an oligoclonal antibody, a polyclonal antibody, a monoclonal antibody (including full-length monoclonal antibodies), a camelised antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species. Native full length antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The term “variable region” may also be used to describe the variable domain of a heavy chain or light chain. Particular amino acid residues are believed to form an interface between the light and heavy chain variable domains. The variable regions of each light/heavy chain pair form an antibody binding site. Such antibodies may be derived from any mammal, including, but not limited to, humans, monkeys, pigs, horses, rabbits, dogs, cats, mice, etc.

The term “antigen binding fragment” includes binding fragments of the antibodies of the disclosure, exemplary fragments include single-chain Fvs (scFv), single-chain antibodies, single domain antibodies, domain antibodies, Fv fragments, Fab fragments, F(ab′) fragments, F(ab′)2 fragments, antigen binding fragments that exhibit the desired biological activity, disulfide-stabilised variable region (dsFv), dimeric variable region (Diabody), anti-idiotypic (anti-Id) antibodies (including, e.g., anti-Id antibodies to antibodies of the disclosure), intrabodies, linear antibodies, single-chain antibody molecules and multispecific antibodies formed from antigen binding fragments and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass. An “antibody” or “antigen binding fragment” of the invention can, for example, inhibit at least one of the biological activities of CXCR4, as discussed above.

It has been shown that fragments of a whole antibody can perform the function of binding antigens. Examples of binding fragments are (Ward, E. S. et al., (1989) Nature 341, 544-546) the Fab fragment consisting of VL, VH, CL and CH1 domains; (McCafferty et al (1990) Nature 348, 552-554) the Fd fragment consisting of the VH and CH1 domains; (Holt et al (2003) Trends in Biotechnology 21, 484-490) the Fv fragment consisting of the VL and VH domains of a single antibody; (iv) the dAb fragment (Ward, E. S. et al., Nature 341, 544-546 (1989), McCafferty et al (1990) Nature 348, 552-554, Holt et al (2003) Trends in Biotechnology 21, 484-490], which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, (1988) Science 242, 423-426, Huston et al, (1988) PNAS USA, 85, 5879-5883); (viii) bispecific single chain Fv dimers (PCT/US92/09965) and (ix) “diabodies”, multivalent or multispecific fragments constructed by gene fusion (WO94/13804; Holliger, P. (1993) et al, Proc. Natl. Acad. Sci. USA 90 6444-6448). Fv, scFv or diabody molecules may be stabilised by the incorporation of disulphide bridges linking the VH and VL domains (Reiter, Y. et al, Nature Biotech 14, 1239-1245, 1996). Minibodies comprising a scFv joined to a CH3 domain may also be made (Hu, S. et al, (1996) Cancer Res. 56, 3055-3061). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991)). The constant domains are generally not involved directly in antigen binding, but may influence antigen binding affinity and may exhibit various effector functions, such as participation of the antibody in ADCC, CDC, and/or apoptosis.

The term “patient” or “subject” includes human and veterinary subjects.

The term “mAb” refers to monoclonal antibody.

The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, complementarity determining region (CDRs) and framework regions (FR), of an antibody follow, unless otherwise indicated, the Kabat definition as set forth in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or CDR of the variable domain. For example, a heavy chain variable domain may include a single amino acid insertion (residue 52a according to Kabat) after residue 52 of H2 and inserted residues (e.g. residues 82a, 82b, and 82c, etc according to Kabat) after heavy chain FR residue 82. The Kabat numbering of residues may be determined for a given antibody by alignment at regions of homology of the sequence of the antibody with a “standard” Kabat numbered sequence. Maximal alignment of framework residues frequently requires the insertion of “spacer” residues in the numbering system, to be used for the Fv region. In addition, the identity of certain individual residues at any given Kabat site number may vary from antibody chain to antibody chain due to interspecies or allelic divergence.

### (ii) Anti-CXCR4 Antibodies

The present disclosure provides anti-CXCR4 antibodies or antigen binding fragments that specifically bind to human CXCR4 and inhibit one or more activities of human CXCR4. In this section of the specification, functional and structural characteristics of exemplary CXCR4 antibodies or antigen binding fragments of the disclosure are described in detail. It should be understood that antibodies or antigen binding fragments of the disclosure can be described based on any one or more (2, 3, 4, 5, 6, 7, 8, 9, etc.) of the structural and/or functional characteristics described herein. Throughout this portion of the disclosure, when a functional or structural characteristic is described with respect to antibodies of the disclosure, it should be understood that, except where context clearly indicates otherwise, such structural or functional characteristic may similarly be used to describe an antigen binding fragment of the disclosure.

### Effects of Inhibiting CXCR4

Embodiments of the disclosure relate to antibodies that specifically bind to CXCR4 and inhibit a biological activity of CXCR4, such as tumor growth or survival. In one embodiment an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the biological activity then would occur in the absence of an antibody of the disclosure. In one example, an antibody of the disclosure inhibits breast cancer tumor growth in SCID xenograft models. In this example, the antibody of the disclosure, such as 6C7, 4C1, 2A4, 5C9, 5E1, or 7C8 (or an antibody comprising the VH and/or VL domains, the 6 CDRs, or a CDR3 of any of 6C7, 4C1, 2A4, 5C9, 5E1, or 7C8), can reduce tumor growth of MDA-MB-231 by over 50%. In another example, the antibodies of the disclosure can be used to treat ovarian cancer by inhibiting the growth of ovarian tumors. In this example, an antibody of the disclosure, such as 6C7, 4C1, 2A4, 5C9, 5E1, or 7C8 (or an antibody comprising the VH and/or VL domains, the 6 CDRs, or a CDR3 of any of 6C7, 4C1, 2A4, 5C9, 5E1, or 7C8), may reduce tumor growth by, for example, over 40%. In yet another example, an antibody of the disclosure can be used to treat B-cell lymphoma. In this example, an antibody of the disclosure, such as 6C7, 4C1, 2A4, 5C9, 5E1, or 7C8 (or an antibody comprising the VH and/or VL domains, the 6 CDRs, or a CDR3 of any of 6C7, 4C1, 2A4, 5C9, 5E1, or 7C8), can be used to inhibit tumor growth by, for example, over 45%. In certain embodiments, an antibody of the disclosure is an antibody that specifically binds to CXCR4, such as an antibody (or antigen binding fragment) having the heavy and/or light chain CDRs (CDR1, CDR2, CDR3) of any of the antibodies described herein, or an antibody (or fragment) having a VH and/or VL chain amino acid sequence of any of the antibodies described herein.

Embodiments of the disclosure relate to antibodies that specifically bind to human CXCR4 and thereby inhibit human CXCR4 activity. In one embodiment, an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of CXCR4 activity then would occur in the absence of an antibody of the disclosure. In certain embodiments, an antibody of the disclosure is an antibody that specifically binds to CXCR4, such as an antibody (or antigen binding fragment thereof) having the heavy and/or light chain CDRs of any of the antibodies described herein, or an antibody (or antigen binding fragment thereof) having a VH and/or VL chain amino acid sequence of any of the antibodies described herein.

Embodiments of the disclosure relate to antibodies that specifically bind to human CXCR4 and thereby inhibit SDF-1 binding activity. In one embodiment, an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% binding of SDF-1 to human CXCR4 transfected HEK293T cells then would occur in the absence of an antibody of the disclosure. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

Embodiments of the disclosure relate to antibodies that specifically bind to CXCR4 and inhibit SDF-1 induced tumor proliferation mediated via CXCR4. In one embodiment, an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of SDF-1 induced tumor proliferation then would occur in the absence of an antibody of the disclosure. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

Further embodiments of the disclosure relate to antibodies that specifically bind to CXCR4 and thereby inhibit SDF-1 induced tumor cell survival. In one embodiment, an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of SDF-1 induced tumor cell survival then would occur in the absence of an antibody of the disclosure. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

Further embodiments of the disclosure relate to antibodies that specifically bind to CXCR4 and thereby inhibit SDF-1 induced cellular metastasis. In one embodiment, an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of SDF-1 cellular metastasis then would occur in the absence of an antibody of the disclosure. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

Further embodiments of the disclosure relate to antibodies that specifically bind to CXCR4 and thereby inhibit phosphorylation of phosphor-MAP kinase including Erk1 and Erk2 and/or AKT kinase. In one embodiment, an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or 100% of the phosphorylation of Erk and/or AKT kinase then would occur in the absence of an antibody of the disclosure. In one embodiment, in Jurkat cells, an antibody of the disclosure specifically binds to CXCR4 and inhibits SDF-1 induced phosphorylation of Erk1 and/or Erk2 by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% then would occur in the absence of an antibody of the disclosure. In another embodiment, in MDA-MB-231 cells, an antibody of the disclosure specifically binds to CXCR4 and inhibits SDF-1 induced phosphorylation of AKT by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% then would occur in the absence of an antibody of the disclosure. In this embodiment, antibodies of the disclosure do not show significant inhibition of Erk1 or Erk2 phosphorylation in MDA-MB-231 cells. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

In one embodiment, antibodies of the disclosure inhibit SDF-1 induced MAP kinase phosphorylation. In one example, an antibody of the disclosure inhibits SDF-1 induced MAP kinase phosphorylation in Jurkat cells with an IC50 of less than 5 nM, e.g., 4 nM, 3.5 nM, 3.0 nM, 2 nM, or 1 nM. In another example, 6C7 inhibits SDF-1 induced MAP kinase phosphorylation in Jurkat cells with an IC50 of less than 3.5 nM.

Further embodiments of the disclosure relate to antibodies that specifically bind to CXCR4 and thereby inhibit cell proliferation in response to SDF-1 ligand. In one embodiment, an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of cell proliferation that would occur in the absence of an antibody of the disclosure. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

Further embodiments of the disclosure relate to antibodies that specifically bind to CXCR4 and induce apoptosis in cells expressing CXCR4. In one embodiment, an antibody of the disclosure induces at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of cellular apoptosis then would occur in the absence of an antibody of the disclosure. In one embodiment, an antibody of the disclosure induces apoptosis in Ramos cells by between 10-70%, 30-60%, or 20-40%. In one example, 6C7, 4C1, 2A4, 5C9, 5E1, or 7C8 (or an antibody comprising the VH and/or VL domains, the 6 CDRs, or a CDR3 of any of 6C7, 4C1, 2A4, 5C9, 5E1, or 7C8) induces apoptosis in Ramos cells by between 20-40%. In another example, 6C7, 4C1, 2A4, 5C9, 5E1, or 7C8 (or an antibody comprising the VH and/or VL domains, the 6 CDRs, or a CDR3 of any of 6C7, 4C1, 2A4, 5C9, 5E1, or 7C8) includes apoptosis in Ramos cells by between 30-60%. In a third example, 6C7, 4C1, 2A4, 5C9, 5E1, or 7C8 (or an antibody comprising the VH and/or VL domains, the 6 CDRs, or a CDR3 of any of 6C7, 4C1, 2A4, 5C9, 5E1, or 7C8) induces apoptosis in HUVEC cells by between 40-60%. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

Further embodiments of the disclosure relate to antibodies that specifically bind to CXCR4 and inhibit Jurkat chemotaxis. In one embodiment, an antibody of the disclosure inhibits Jurkat chemotaxis at an IC50 concentration (a concentration to achieve 50% inhibition of) of below 10 nM, e.g., 5 nM, 4 nM, 3 nM, 2 nM, 1 nM, 0.6 nM, 0.5 nM, 0.4 nM, 0.3 nM, 0.2 nm, 0.1 nM, 0.09, 0.08, 0.07, 0.06, 0.05, or 0.01 nM. For example, in one embodiment, an antibody of the disclosure inhibits Jurkat chemotaxis at an IC50 concentration (a concentration to achieve 50% inhibition of) of between 0.01 nM to 1 nM. In yet another embodiment, an antibody of the disclosure inhibits Jurkat chemotaxis at an IC50 concentration (a concentration to achieve 50% inhibition of) of below 1500 ng/ml, e.g., 750 ng/ml, 500 ng/ml, 250 ng/ml, 125 ng/ml, 100 ng/ml, 50 ng/ml, 40 ng/ml, 30 ng/ml, 20 ng/ml, or 10 ng/ml. In one embodiment, an antibody of the disclosure inhibits Jurkat chemotaxis at an IC50 of below 185, 150, 90, 80, 70, 60, 50, 40, 30, 20, 19, 18, 17, 16, 15, 14, 13, 12, 11, or 10 ng/ml. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

Further embodiments of the disclosure relate to antibodies that specifically bind to CXCR4 and inhibit migration of HUVECs in a scratch-wound healing assay. In one embodiment, an antibody of the disclosure inhibits HUVEC migration at an IC50 concentration (a concentration to achieve 50% inhibition of) of below 10 nM, e.g., 5 nM, 4 nM, 3 nM, 2 nM, 1 nM, 0.5 nM, 0.1 nM, or 0.01 nM. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

Further embodiments of the disclosure relate to antibodies that curtail a reduction in B-cell counts, for example, an antibody of the disclosure can cause no more than a 60% reduction of B-cell counts when added to a peripheral blood leukocyte cell preparation at a concentration of 10 ug/ml over a period of 16-18 hours. In particular embodiments the antibody can cause no more than a 50% reduction of B-cell counts when added to a peripheral blood leukocyte cell preparation at a concentration of 10 ug/ml over a period of 16-18 hours. In certain embodiments, an antibody of the disclosure is an antibody that specifically binds to CXCR4, such as an antibody (or fragment) having the heavy and/or light chain CDRs of any of the antibodies described herein, or an antibody (or fragment) having a VH and/or VL chain amino acid sequence of any of the antibodies described herein.

### Functional Characteristics of Antibodies

A further embodiment of the disclosure is an antibody which competes for binding to CXCR4 with an antibody of the disclosure. In another embodiment, an antibody of the disclosure competes for binding to CXCR4 with any one of fully human monoclonal antibodies described herein including 6C7, 2A4 or 4C1 or an antibody comprising an amino acid sequence of the VH and VL domains of any of the foregoing antibodies. “Competes” indicates that an antibody competes for binding to CXCR4 with any one of fully human monoclonal antibodies 6C7, 2A4 or 4C1, i.e. competition is unidirectional.

Embodiments of the disclosure include antibodies which cross compete with any one of fully human monoclonal antibodies described herein including 6C7, 2A4, 4C1, 5C9, 5E1 or 7C8) or an antibody comprising an amino acid sequence of the VH and VL domains of any of the foregoing antibodies for binding to CXCR4. “Cross competes” indicates that the antibody competes for binding to CXCR4 with any one of fully human monoclonal antibodies described herein including 6C7, 2A4 or 4C1, and vice versa, i.e. competition is bidirectional. “Cross competes” also refers to, for example, the ability of one anti-CXCR4 antibody or antigen binding fragment to inhibit or neutralize the biological activity of CXCR4, as discussed above, to a similar extent as another anti-CXCR4 antibody or antigen binding fragment.

A further embodiment of the disclosure is an antibody or antigen binding fragment that binds to the same epitope or epitopes on CXCR4 as an antibody of the disclosure. Embodiments of the disclosure also include an antibody or antigen binding fragment that binds to the same epitope or epitopes on CXCR4 as any one of fully human monoclonal antibodies described herein including 6C7, 2A4 or 4C1 or an antibody comprising an amino acid sequence of the VH and VL domains of any of the foregoing antibodies. Certain embodiments of the disclosure include an antibody or antigen binding fragment that binds to overlapping epitope(s) of two or more antibodies of the invention (e.g., 6C7, 4C1, 2A4, 5C9, 5E1, or 7C8).

In one embodiment, the antibody is a bispecific antibody. A bispecific antibody is an antibody that has binding specificity for at least two different epitopes on the same or on different proteins. Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; and EP 03089.)

Embodiments of the disclosure described herein relate to monoclonal antibodies that specifically bind CXCR4 and affect CXCR4 function. Other embodiments relate to fully human antibodies that specifically bind CXCR4 and preparations thereof with desirable properties from a therapeutic perspective, including high binding affinity for CXCR4, high selectivity for inhibition of CXCR4 signaling, low toxicity, the ability to block SDF-1 binding to CXCR4, the ability to inhibit CXCR4-induced proliferative, angiogenic, cell adhesion or invasion-related diseases include neoplastic diseases, and/or the ability to inhibit tumour cell growth in vitro and in vivo. Still other embodiments relate to fully human antibodies that specifically bind CXCR4 and preparations thereof that do not result in a significant Human Anti-Chimeric Antibody (HACA) response, thereby allowing for repeated administration.

### Specificity of CXCR4 Inhibition

Antibodies of the disclosure bind human CXCR4. In some examples, an antibody of the disclosure is cross-reactive with CXCR4 proteins from other species. In one embodiment, an antibody of the disclosure is cross-reactive with CXCR4 from a non-human primate. In one embodiment, an antibody of the disclosure is cross-reactive with a non-human primate such as cynomolgus monkey CXCR4. In another embodiment, an antibody of the disclosure is cross-reactive with a non-human primate such as cynomolgus monkey CXCR4 but is only weakly cross-reactive or shows no cross-reactivity with CXCR4 proteins from other species, e.g., no cross reactivity was detected with native mouse CXCR4. In one embodiment, an antibody of the disclosure binds CXCR4 molecules from non-human primate such as cynomolgus monkey with high affinity, e.g., a Kd of less than 1 nM.

In another embodiment, an antibody of the disclosure is specific for CXCR4 and does not crossreact with other chemokine receptor members. In one example, an antibody of the disclosure does not cross react with CXCR3 and/or CCR4.

In yet another embodiment, an antibody of the disclosure inhibits SDF-1 ligand binding to the CXCR4 receptor. In one example, activity possessed by the antibody can be demonstrated at an IC50 concentration (a concentration to achieve 50% inhibition of) below 10 μM. In another example, an antibody of the disclosure can have an IC50 concentration of less than 50, 40, 30, 20, 10, 5, 4 or 2 nM.

Antibodies described herein can have at least one of the activities as described above. In one embodiment, an antibody of the disclosure can inhibit SDF-1 ligand binding to the CXCR4 receptor by above 70% and further can inhibit Jurkat cell chemotaxis by at least 80% when the assay is run for 24 hours. In another embodiment, an antibody of the disclosure can inhibit SDF-1 ligand binding to the CXCR4 receptor by between 20-60% and further can inhibit Jurkat cell chemotaxis by at least 80% when the assay is run for 24 hours. In another embodiment, an antibody of the disclosure does not inhibit SDF-1 ligand binding to the CXCR4 receptor but can still inhibit Jurkat cell chemotaxis by at least 80%.

A further embodiment is an antibody that specifically binds to CXCR4 and comprises a sequence comprising one or more (1, 2, 3, 4, 5, 6) of the complementarity determining regions (CDR) sequences shown in Table 7 and/or Table 8. Embodiments of the disclosure include an antibody comprising a sequence comprising: any one of a CDR1, a CDR2 or a CDR3 sequence as shown in Table 7. A further embodiment is an antibody that specifically binds to CXCR4 and comprises a sequence comprising any two of the CDR sequences shown in Table 7. In another embodiment, an antibody comprises a sequence comprising a CDR1, a CDR2 and a CDR3 sequence as shown in Table 7. In another embodiment, an antibody comprises a sequence comprising one or more of the CDR sequences shown in Table 8. Embodiments of the disclosure include an antibody comprising a sequence comprising: any one of a CDR1, a CDR2 or a CDR3 sequence as shown in Table 8. In another embodiment the antibody comprises a sequence comprising any two of the CDR sequences shown in Table 8. In another embodiment the antibody comprises a sequence comprising a CDR1, a CDR2 and a CDR3 sequence as shown in Table 8. In another embodiment the antibody may comprise a sequence comprising a CDR1, a CDR2 and a CDR3 sequence as shown in Table 7 and a CDR1, a CDR2 and a CDR3 sequence as shown in Table 8. In certain embodiments, the antibody is a fully human monoclonal antibody. In certain other embodiments, the antibody is a binding fragment of a fully human monoclonal antibody.

A further embodiment is an antibody that specifically binds to CXCR4 and comprises a sequence comprising one of the CDR3 sequences shown in Table 7. A further embodiment is an antibody that specifically binds to CXCR4 and comprises a sequence comprising one of the CDR3 sequences shown in Table 8. In another embodiment, the antibody may comprise a sequence comprising a CDR3 sequence as shown in Table 7 and a CDR3 sequence as shown in Table 8.

A further embodiment is an antibody that specifically binds to CXCR4 and comprises a sequence comprising one of the CDR3 sequences shown in Table 7. In a further embodiment, the antibody further comprises a sequence comprising: a CDR3 sequence as shown in Table 8. In a further embodiment, the antibody further comprises a sequence comprising: a CDR2 and a CDR3 sequence as shown in Table 7. In a further embodiment, the antibody further comprises a sequence comprising: a CDR1, a CDR2 and a CDR3 sequence as shown in Table 7.

A further embodiment is an antibody that specifically binds to CXCR4 and comprises a sequence comprising one of the CDR2 and one of the CDR3 sequences shown in Table 7. In a further embodiment, the antibody further comprises a sequence comprising: a CDR3 sequence as shown in Table 8. In a further embodiment, the antibody further comprises a sequence comprising: a CDR1, a CDR2 and a CDR3 sequence as shown in Table 7.

A further embodiment is an antibody that specifically binds to CXCR4 and comprises a sequence comprising one of the CDR2 and one of the CDR3 sequences shown in Table 7. In a further embodiment, the antibody further comprises a sequence comprising: a CDR1, a CDR2 and a CDR3 sequence as shown in Table 7.

A further embodiment is an antibody that specifically binds to CXCR4 and comprises a sequence comprising one of the CDR2 and one of the CDR3 sequences shown in Table 8. In a further embodiment, the antibody further comprises a sequence comprising: a CDR1, a CDR2 and a CDR3 sequence as shown in Table 8.

It is noted that those of ordinary skill in the art can readily accomplish CDR determinations. See for example, Kabat et al., Sequences of Proteins of Immunological Interest, Fifth Edition, NIH Publication 91-3242, Bethesda Md. (1991), vols. 1-3. Kabat provides multiple sequence alignments of immunoglobulin chains from numerous species antibody isotypes. The aligned sequences are numbered according to a single numbering system, the Kabat numbering system. The Kabat sequences have been updated since the 1991 publication and are available as an electronic sequence database (presently available from the Kabat Database Website; see also Nucleic Acids Research, 2000, 28(1), 214-218). Any immunoglobulin sequence can be numbered according to Kabat by performing an alignment with the Kabat reference sequence. Accordingly, the Kabat numbering system provides a uniform system for numbering immunoglobulin chains.

### Antibody Structure

The basic structural unit of native antibodies is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antigen binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Bispecific antibodies do not exist in the form of fragments having a single binding site (e.g., Fab, Fab′, and Fv).

Typically, a VH domain is paired with a VL domain to provide an antibody antigen-binding site, although a VH or VL domain alone may be used to bind antigen. The VH domain (see Table 7) may be paired with the VL domain (see Table 8), so that an antibody antigen-binding site is formed comprising both a VH and a VL domain.

In certain embodiments of the disclosure, the antibody is a monoclonal antibody. In other embodiments of the disclosure, the antibody is a fully human monoclonal antibody.

Antibodies, monoclonal antibodies and human monoclonal antibodies include the antibodies of the IgG1, IgG2, IgG3 and IgG4 isotypes, for example IgG2. In one embodiment of the disclosure, the antibody is a fully human monoclonal antibody of the IgG2 isotype. This isotype has reduced potential to elicit effector function in comparison with other isotypes, which may lead to reduced toxicity. In another embodiment of the disclosure, the antibody is a fully human monoclonal antibody of the IgG1 isotype. The IgG1 isotype has increased potential to elicit ADCC in comparison with other isotypes, which may lead to improved efficacy. The IgG1 isotype has improved stability in comparison with other isotypes, e.g. IgG4, which may lead to improved bioavailability/ease of manufacture/longer half-life. In one embodiment, the fully human monoclonal antibody of the IgG1 isotype is of the z, za or f allotype. In another embodiment of the disclosure, the antibody is a fully human monoclonal antibody of the IgG1 isotype, with mutations introduced in the Fc region to minimize Fc receptor binding and engagement of effector function. In another embodiment of the disclosure, the antibody has desirable therapeutic properties, selected from one or more of high binding affinity for CXCR4, the ability to inhibit CXCR4 activity in vitro and in vivo, and the ability to inhibit CXCR4-induced cell adhesion, proliferation, motility, invasion, metastasis, tumour growth and angiogenesis.

In one embodiment, the disclosure includes antibodies that specifically bind to CXCR4 with very high affinities (Kd). In some embodiments of the disclosure, the antibody binds CXCR4 with a binding affinity (Kd) of less than 5 nanomolar (nM). In other embodiments, the targeted binding agent binds with a Kd of less than 4 nM, 3 nM, 2.5 nM, 2 nM or 1 nM. In some embodiments of the disclosure, the antibody binds CXCR4 with a Kd of less than 950 picomolar (pM). In some embodiments of the disclosure, the antibody binds CXCR4 with a Kd of less than 900 pM. In other embodiments, the antibody binds CXCR4 with a Kd of less than 800 pM, 700 pM or 600 pM. In some embodiments of the disclosure, the antibody binds CXCR4 with a Kd of less than 500 pM. In other embodiments, the antibody binds CXCR4 with a Kd of less than 400 pM. In still other embodiments, the antibody binds CXCR4 with a Kd of less than 300 pM. In some other embodiments, the antibody binds CXCR4 with a Kd of less than 200 pM. In some other embodiments, the antibody binds CXCR4 with a Kd of less than 100 pM. In some other embodiments, the antibody binds CXCR4 with a Kd of less than 90 pM, 80 pM, 70 pM, 60 pM, 55 pM or 50 pM. In some other embodiments, the antibody binds CXCR4 with a Kd of less than 60 pM. In some other embodiments, the antibody binds CXCR4 with a Kd of less than 55 pM. The Kd may be assessed using a method described herein or known to one of skill in the art (e.g., a BIAcore assay, ELISA) (Biacore International AB, Uppsala, Sweden). In one embodiment, the antibodies of the disclosure bind human CXCR4 with a K_{D} of less than 2.5 nM, 2.0 nM, 1.5 nM, 1 nM, 0.5 nM when measured by FACS binding kinexa analysis. Antibodies of the disclosure have considerably improved binding affinities for CXCR4 in comparison with the antibodies reported in the prior art.

The binding properties of antibodies of the disclosure may also be measured by reference to the dissociation or association rates (k_{off} and kₒₙ respectively).

In one embodiment of the disclosure, an antibody may have an kₒₙ rate (antibody (Ab) +antigen_{(Ag)kw→Ab-Ag) of at least} 10⁴ M⁻¹s⁻¹, at least 5×10⁴ M⁻¹s⁻¹, at least 10⁵ M⁻¹s⁻¹, at least 2×10⁵ M⁻¹s⁻¹, at least 5×10⁵M⁻¹s⁻¹, at least 10⁶ M⁻¹s⁻¹, at least 5×10⁶ M⁻¹s⁻¹, at least 10⁷ M⁻¹s⁻¹, at least 5×10⁷M⁻¹s⁻¹, or at least 10⁸M⁻s⁻¹.

In another embodiment of the disclosure, an antibody may have a k_{off} rate ((Ab—Ag)^{k}^{_{off}}→antibody (Ab)+antigen (Ag)) of less than 5×10⁻¹ s⁻¹, less than 10⁻¹ s⁻¹, less than 5×10⁻² s⁻¹, less than 10⁻² s⁻¹ less than 5×10⁻³ s⁻¹, less than 10⁻³ s⁻¹, less than 5×10⁻⁴ s⁻¹, less than 10⁻⁴ s⁻¹, less than 5×10⁻⁵ s⁻¹, less than 10⁻⁵ s⁻¹, less than 5×10⁻⁶ s⁻¹, less than 10⁻⁶ s⁻¹, less than 5×10⁻⁷ s¹, less than 10⁻⁷ s⁻¹, less than 5×10⁻⁸s⁻¹, less than 10⁻⁸s⁻¹, less than 5×10⁻⁹ s⁻¹, less than 10⁻⁹ s⁻¹, or less than 10⁻¹⁰ s⁻¹.

Embodiments of the disclosure include the antibodies listed below in Table 1. This table reports the identification number of each antibody, along with the SEQ ID number of the variable domain of the corresponding heavy chain and light chain genes and polypeptides, respectively. Each antibody has been given an identification number.

TABLE 1

| mAb ID No.: | Sequence | SEQ ID NO: |
|---|---|---|
| 4C1 | Nucleotide sequence encoding the | 1 |
| | variable region of the heavy chain | |
| | Amino acid sequence encoding the | 2 |
| | variable region of the heavy chain | |
| | Nucleotide sequence encoding the | 3 |
| | variable region of the light chain | |
| | Amino acid sequence encoding the | 4 |
| | variable region of the light chain | |
| 6C7 | Nucleotide sequence encoding the | 5 |
| | variable region of the heavy chain | |
| | Amino acid sequence encoding the | 6 |
| | variable region of the heavy chain | |
| | Nucleotide sequence encoding the | 7 |
| | variable region of the light chain | |
| | Amino acid sequence encoding the | 8 |
| | variable region of the light chain | |
| 2A4 | Nucleotide sequence encoding the | 9 |
| | variable region of the heavy chain | |
| | Amino acid sequence encoding the | 10 |
| | variable region of the heavy chain | |
| | Nucleotide sequence encoding the | 11 |
| | variable region of the light chain | |
| | Amino acid sequence encoding the | 12 |
| | variable region of the light chain | |
| 5C9 | Nucleotide sequence encoding the | 13 |
| | variable region of the heavy chain | |
| | Amino acid sequence encoding the | 14 |
| | variable region of the heavy chain | |
| | Nucleotide sequence encoding the | 15 |
| | variable region of the light chain | |
| | Amino acid sequence encoding the | 16 |
| | variable region of the light chain | |
| 5E1 | Nucleotide sequence encoding the | 17 |
| | variable region of the heavy chain | |
| | Amino acid sequence encoding the | 18 |
| | variable region of the heavy chain | |
| | Nucleotide sequence encoding the | 19 |
| | variable region of the light chain | |
| | Amino acid sequence encoding the | 20 |
| | variable region of the light chain | |
| 7C8 | Nucleotide sequence encoding the | 21 |
| | variable region of the heavy chain | |
| | Amino acid sequence encoding the | 22 |
| | variable region of the heavy chain | |
| | Nucleotide sequence encoding the | 23 |
| | variable region of the light chain | |
| | Amino acid sequence encoding the | 24 |
| | variable region of the light chain | |

### Exemplary Sequences

In one embodiment, an antibody of the disclosure comprises a sequence comprising any one of the heavy chain sequences (VH) listed in Table 1 or shown in Table 7. In another embodiment, the antibody comprises a sequence comprising any one of the heavy chain sequences of antibodies 2A4, 4C1, 5C9, 5E1, 6C7 or 7C8. Light-chain promiscuity is well established in the art, thus, an antibody comprising a sequence comprising any one of the heavy chain sequences of antibodies 2A4, 4C1, 5C9, 5E1, 6C7 or 7C8, may further comprise any one of the light chain sequences (VL) listed in Table 1 or shown in Table 8 or of antibodies 2A4, 4C1, 5C9, 5E1, 6C7 or 7C8. In another embodiment, an antibody of the disclosure comprises a sequence comprising any one of the heavy chain sequences of antibodies 2A4, 4C1, 5C9, 5E1, 6C7 or 7C8 and further comprises the corresponding light chain sequence of antibody 2A4, 4C1, 5C9, 5E1, 6C7 or 7C8. In some embodiments, the antibody is a fully human monoclonal antibody. In some embodiments, the antibody specifically binds to CXCR4 and comprises a heavy chain and a light chain, wherein the heavy chain comprises the amino acid sequence of SEQ ID NO: 2, 6, 10, 14, 18 or 22.

In one embodiment, the antibody comprises a sequence comprising any one of the light chain sequences shown in Table 8. In another embodiment, the antibody comprises a sequence comprising any one of the light chain sequences of antibodies 2A4, 4C1, 5C9, 5E1, 6C7 or 7C8. In some embodiments, the antibody is a fully human monoclonal antibody. In some embodiments, the antibody specifically binds to CXCR4 and comprises a heavy chain and a light chain, wherein the light chain comprises the amino acid sequence of SEQ ID NO: 4, 8, 12, 16, 20 or 24.

In other embodiments, the antibody specifically binds to CXCR4 and comprises a heavy chain comprising the amino acid sequence of SEQ ID NO: 2, 6, 10, 14, 18 or 22, and a light chain comprising the amino acid sequence of SEQ ID NO: 4, 8, 12, 16, 20 or 24.

In another embodiment, the antibody comprises a sequence comprising any the heavy chain sequence of antibody 4C1 and further comprises the light chain sequence of antibody 4C1. In another embodiment, the antibody comprises a sequence comprising any the heavy chain sequence of antibody 2A4 and further comprising the light chain sequence of antibody 2A4. In another embodiment, the antibody comprises a sequence comprising any the heavy chain sequence of antibody 7C8 and further comprising the light chain sequence of antibody 7C8.

In some embodiments, an antibody of the disclosure is any one of the monoclonal antibodies as shown in Table 1. In some embodiments, the antibody is a monoclonal antibody selected from the group consisting of: 4C1, 2A4 and 6C7. In one embodiment, an antibody of the disclosure comprises one or more of fully human monoclonal antibodies 4C1, 2A4 or 6C7. In certain embodiments, the antibody is monoclonal antibody 4C1. In certain other embodiments, the antibody is monoclonal antibody 2A4. In still other embodiments, the antibody is monoclonal antibody 6C7. In additional embodiments, an antibody of the disclosure is derivable from any of the foregoing monoclonal antibodies.

The variable heavy and the variable light chains of antibodies 4C1, 2A4 and 6C7 were deposited in plasmids at the American Type Culture Collection (ATCC) under the designation names of Mab4C1VH, Mab4C1VL, Mab2A4VH, Mab2A4VL, Mab6C7VH and Mab6C7VL.

In another embodiment, an antibody of the disclosure may comprise a sequence comprising any one, two or three of the CDR1, CDR2 or CDR3 of the heavy chain variable domain sequences encoded by a polynucleotide in a plasmid designated Mab4C1VH, Mab2A4VH, and Mab6C7VH, which were deposited at the American Type Culture Collection (ATCC) under number PTA-9626, PTA-9627, or PTA-9630 on Nov. 18, 2008, or a polynucleotide encoding the same amino acid sequence. In another embodiment, an antibody of the disclosure may comprise a sequence comprising any one, two or three of the CDR1, CDR2 or CDR3 of the variable light chain sequences encoded by a polynucleotide in a plasmid designated Mab4C1VL, Mab2A4VL, and Mab6C7VL which were deposited at the American Type Culture Collection (ATCC) under number PTA-9629, PTA-9628, or PTA-9631 on Nov. 18, 2008, or a polynucleotide encoding the same amino acid sequence, or a polynucleotide encoding the same amino acid sequence.

In one embodiment, an antibody of the disclosure comprises a heavy chain variable domain sequence comprising a CDR3 encoded by the polynucleotide in plasmid designated Mab4C1VH which was deposited at the American Type Culture Collection (ATCC) under number PTA-9626 on Nov. 18, 2008, or a polynucleotide encoding the same amino acid sequence.

In one embodiment, an antibody of the disclosure comprises a heavy chain variable domain sequence comprising a CDR3 encoded by the polynucleotide in plasmid designated Mab4C1VH which was deposited at the American Type Culture Collection (ATCC) under number PTA-9626 on Nov. 18, 2008 and a light chain variable domain sequence comprising a CDR3 encoded by the polynucleotide in plasmid designated Mab4C1VL which was deposited at the American Type Culture Collection (ATCC) under number PTA-9629 on Nov. 18, 2008, or a polynucleotide encoding the same amino acid sequence.

In another embodiment, an antibody of the disclosure comprises a heavy chain variable domain sequence comprising at least one, at least two, or at least three of the CDRs encoded by the polynucleotide in plasmid designated Mab4C1VH which was deposited at the American Type Culture Collection (ATCC) under number PTA-9626 on Nov. 18, 2008, or a polynucleotide encoding the same amino acid sequence.

In another embodiment, an antibody of the disclosure comprises a light chain variable domain sequence comprising at least one, at least two, or at least three of the CDRs encoded by the polynucleotide in plasmid designated Mab4C1VL which was deposited at the American Type Culture Collection (ATCC) under number PTA-9629 on Nov. 18, 2008, or a polynucleotide encoding the same amino acid sequence.

In another embodiment, an antibody of the disclosure comprises a heavy chain variable domain sequence comprising at least one, at least two, or at least three of the CDRs encoded by the polynucleotide in plasmid designated Mab4C1VH which was deposited at the American Type Culture Collection (ATCC) under number PTA-9626 on Nov. 18, 2008, or a polynucleotide encoding the same amino acid sequence, and a light chain variable domain sequence comprising at least one, at least two, or at least three of the CDRs encoded by the polynucleotide in plasmid designated Mab4C1VL which was deposited at the American Type Culture Collection (ATCC) under number PTA-9629 on Nov. 18, 2008, or a polynucleotide encoding the same amino acid sequence.

In one embodiment, an antibody of the disclosure comprises a heavy chain variable domain sequence comprising a CDR3 encoded by the polynucleotide in plasmid designated Mab2A4VH which was deposited at the American Type Culture Collection (ATCC) under number PTA-9627 on Nov. 18, 2008, or a polynucleotide encoding the same amino acid sequence.

In one embodiment, an antibody of the disclosure comprises a heavy chain variable domain sequence comprising a CDR3 encoded by the polynucleotide in plasmid designated Mab2A4VH which was deposited at the American Type Culture Collection (ATCC) under number PTA-9627 on Nov. 18, 2008 and a light chain variable domain sequence comprising a CDR3 encoded by the polynucleotide in plasmid designated Mab2A4VL which was deposited at the American Type Culture Collection (ATCC) under number PTA-9628 on Nov. 18, 2008, or a polynucleotide encoding the same amino acid sequence.

In another embodiment, an antibody of the disclosure comprises a heavy chain variable domain sequence comprising at least one, at least two, or at least three of the CDRs of the antibody encoded by the polynucleotide in plasmid designated Mab2A4VH which was deposited at the American Type Culture Collection (ATCC) under number PTA-9627 on Nov. 18, 2008, or a polynucleotide encoding the same amino acid sequence.

In another embodiment, an antibody of the disclosure comprises a light chain variable domain sequence comprising at least one, at least two, or at least three of the CDRs encoded by the polynucleotide in plasmid designated Mab2A4VL which was deposited at the American Type Culture Collection (ATCC) under number PTA-9628 on Nov. 18, 2008, or a polynucleotide encoding the same amino acid sequence.

In another embodiment, an antibody of the disclosure comprises a heavy chain variable domain sequence comprising at least one, at least two, or at least three of the CDRs encoded by the polynucleotide in plasmid designated Mab2A4VH which was deposited at the American Type Culture Collection (ATCC) under number PTA-9627 on Nov. 18, 2008, or a polynucleotide encoding the same amino acid sequence, and a light chain variable domain sequence comprising at least one, at least two, or at least three of the CDRs encoded by the polynucleotide in plasmid designated Mab2A4VL which was deposited at the American Type Culture Collection (ATCC) under number PTA-9628 on Nov. 18, 2008, or a polynucleotide encoding the same amino acid sequence.

In one embodiment, an antibody of the disclosure comprises a heavy chain variable domain sequence comprising a CDR3 encoded by the polynucleotide in plasmid designated Mab6C7VH which was deposited at the American Type Culture Collection (ATCC) under number PTA-9630 on Nov. 18, 2008, or a polynucleotide encoding the same amino acid sequence.

In one embodiment, an antibody of the disclosure comprises a heavy chain variable domain sequence comprising a CDR3 encoded by the polynucleotide in plasmid designated Mab6C7VH which was deposited at the American Type Culture Collection (ATCC) under number PTA-9630 on Nov. 18, 2008, or a polynucleotide encoding the same amino acid sequence, and a light chain variable domain sequence comprising a CDR3 encoded by the polynucleotide in plasmid designated Mab6C7VL which was deposited at the American Type Culture Collection (ATCC) under number PTA-9631 on Nov. 18, 2008, or a polynucleotide encoding the same amino acid sequence.

In another embodiment, an antibody of the disclosure comprises a heavy chain variable domain sequence comprising at least one, at least two, or at least three of the CDRs encoded by the polynucleotide in plasmid designated Mab6C7VH which was deposited at the American Type Culture Collection (ATCC) under number PTA-9630 on Nov. 18, 2008, or a polynucleotide encoding the same amino acid sequence.

In another embodiment, an antibody of the disclosure comprises a light chain variable domain sequence comprising at least one, at least two, or at least three of the CDRs encoded by the polynucleotide in plasmid designated Mab6C7VL which was deposited at the American Type Culture Collection (ATCC) under number PTA-9631 on Nov. 18, 2008, or a polynucleotide encoding the same amino acid sequence.

In another embodiment, an antibody of the disclosure comprises a heavy chain variable domain sequence comprising at least one, at least two, or at least three of the CDRs encoded by the polynucleotide in plasmid designated Mab6C7VH which was deposited at the American Type Culture Collection (ATCC) under number PTA-9630 on Nov. 18, 2008, or a polynucleotide encoding the same amino acid sequence, and a light chain variable domain sequence comprising at least one, at least two, or at least three of the CDRs encoded by the polynucleotide in plasmid designated Mab6C7VL which was deposited at the American Type Culture Collection (ATCC) under number PTA-9631 on Nov. 18, 2008, or a polynucleotide encoding the same amino acid sequence.

In another embodiment, an antibody of the disclosure comprises a heavy chain variable sequence of an antibody encoded by the polynucleotide in plasmid designated Mab4C1VH which was deposited at the American Type Culture Collection (ATCC) under number PTA-9626 on Nov. 18, 2008, or a polynucleotide encoding the same amino acid sequence.

In another embodiment, an antibody of the disclosure comprises a heavy chain variable sequence of an antibody encoded by the polynucleotide in plasmid designated Mab2A4VH which was deposited at the American Type Culture Collection (ATCC) under number PTA-9627 on Nov. 18, 2008, or a polynucleotide encoding the same amino acid sequence.

In another embodiment, an antibody of the disclosure comprises a heavy chain variable sequence of an antibody encoded by the polynucleotide in plasmid designated Mab6C7VH which was deposited at the American Type Culture Collection (ATCC) under number PTA-9630 on Nov. 18, 2008, or a polynucleotide encoding the same amino acid sequence.

In another embodiment, an antibody of the disclosure comprises a variable light chain of an antibody encoded by the polynucleotide in plasmid designated Mab4C1VL which was deposited at the American Type Culture Collection (A TCC) under number PTA-9629 on Nov. 18, 2008, or a polynucleotide encoding the same amino acid sequence.

In another embodiment, an antibody of the disclosure comprises a variable light chain of an antibody encoded by the polynucleotide in plasmid designated Mab2A4VL which was deposited at the American Type Culture Collection (ATCC) under number PTA-9628 on Nov. 18, 2008, or a polynucleotide encoding the same amino acid sequence.

In another embodiment, an antibody of the disclosure comprises a variable light chain of an antibody encoded by the polynucleotide in plasmid designated Mab6C7VL which was deposited at the American Type Culture Collection (A TCC) under number PTA-9631 on Nov. 18, 2008, or a polynucleotide encoding the same amino acid sequence.

In another embodiment, an antibody of the disclosure comprises a heavy chain variable sequence of an antibody encoded by the polynucleotide in plasmid designated Mab4C1VH which was deposited at the American Type Culture Collection (ATCC) under number PTA-9626 on Nov. 18, 2008, or a polynucleotide encoding the same amino acid sequence, and a variable light chain of an antibody encoded by the polynucleotide in plasmid designated Mab4C1VL which was deposited at the American Type Culture Collection (ATCC) under number PTA-9629 on Nov. 18, 2008, or a polynucleotide encoding the same amino acid sequence.

In another embodiment, an antibody of the disclosure comprises a variable light chain of an antibody encoded by the polynucleotide in plasmid designated Mab2A4VL which was deposited at the American Type Culture Collection (ATCC) under number PTA-9628 on Nov. 18, 2008, or a polynucleotide encoding the same amino acid sequence, and a heavy chain variable sequence of an antibody encoded by the polynucleotide in plasmid designated Mab2A4VH which was deposited at the American Type Culture Collection (ATCC) under number PTA-9627 on Nov. 18, 2008, or a polynucleotide encoding the same amino acid sequence.

In another embodiment, an antibody of the disclosure comprises a heavy chain variable sequence of an antibody encoded by the polynucleotide in plasmid designated Mab6C7VH which was deposited at the American Type Culture Collection (ATCC) under number PTA-9630 on Nov. 18, 2008, or a polynucleotide encoding the same amino acid sequence, and a variable light chain of an antibody encoded by the polynucleotide in plasmid designated Mab6C7VL which was deposited at the American Type Culture Collection (A TCC) under number PTA-9631 on Nov. 18, 2008, or a polynucleotide encoding the same amino acid sequence.

In certain embodiments, an antibody of the disclosure may comprise a sequence comprising a heavy chain CDR1 (HCDR1), heavy chain CDR2 (HCDR2) and heavy chain CDR3 (HCDR3) selected from any one of the sequences shown in Table 7. In other embodiments, an antibody of the disclosure may comprise a sequence comprising a light chain CDR1 (LCDR1), light chain CDR2 (LCDR2) and light chain CDR3 (LCDR3) selected from any one of the sequences shown in Table 8. In other embodiments, an antibody of the disclosure may comprise a sequence comprising a HCDR1, HCDR2 and HCDR3 selected from any one of the CDRs of antibodies 2A4, 4C1, 5C9, 5E1, 6C7 or 7C8. In another embodiment, an antibody of the disclosure may comprise a sequence comprising a LCDR1, LCDR2 and LCDR3 selected from any one of the CDRs of antibodies 2A4, 4C1, 5C9, 5E1, 6C7 or 7C8.

In another embodiment, an antibody of the disclosure may comprise a sequence comprising any one of a CDR1, a CDR2 or a CDR3 of any one of the fully human monoclonal antibodies 4C1, 2A4 or 6C7, as shown in Table 7. In another embodiment, an antibody of the disclosure may comprise a sequence comprising any one of a CDR1, a CDR2 or a CDR3 of any one of the fully human monoclonal antibodies 4C1, 2A4 or 6C7, as shown in Table 8. In another embodiment, an antibody of the disclosure may comprise a sequence comprising a CDR1, a CDR2 and a CDR3 of any one of fully human monoclonal antibodies 4C1, 2A4 or 6C7, as shown in Table 7. In another embodiment, an antibody of the disclosure may comprise a sequence comprising a CDR1, a CDR2 and a CDR3 of any one of fully human monoclonal antibodies 4C1, 2A4 or 6C7, as shown in Table 8. In another embodiment, an antibody of the disclosure may comprise a sequence comprising a CDR1, a CDR2 and a CDR3 of any one of fully human monoclonal antibodies 4C1, 2A4 or 6C7, as shown in Table 7, and a CDR1, a CDR2 and a CDR3 sequence of any one of fully human monoclonal antibodies 4C1, 2A4 or 6C7, as shown in Table 8. In some embodiments, the antibody is a fully human monoclonal antibody.

In another embodiment, an antibody of the disclosure comprises a sequence comprising the CDR1, CDR2 and CDR3 sequence of fully human monoclonal antibody 4C1 as shown in Table 7 and the CDR1, CDR2 and CDR3 sequence of fully human monoclonal antibody 4C1 as shown in Table 8. In another embodiment, an antibody of the disclosure comprises a sequence comprising the CDR1, CDR2 and CDR3 sequence of fully human monoclonal antibody 2A4 as shown in Table 7 and the CDR1, CDR2 and CDR3 sequence of fully human monoclonal antibody 2A4 as shown in Table 8. In another embodiment, an antibody of the disclosure comprises a sequence comprising the CDR1, CDR2 and CDR3 sequence of fully human monoclonal antibody 6C7 as shown in Table 7 and the CDR1, CDR2 and CDR3 sequence of fully human monoclonal antibody 6C7 as shown in Table 8. In some embodiments, antibody is a fully human monoclonal antibody.

A further embodiment of the disclosure is antibodies comprising a sequence comprising the contiguous sequence spanning the framework regions and CDRs, specifically from FR1 through FR4 or CDR1 through CDR3, of any one of the sequences as shown in Table 7 or Table 8. A further embodiment of the disclosure is antibodies comprising a sequence comprising the contiguous sequence spanning the framework regions and CDRs, specifically from FR1 through FR4 or CDR1 through CDR3, of any one of the sequences as shown in Table 7 and Table 8. In one embodiment, an antibody of the disclosure comprises a sequence comprising the contiguous sequences spanning the framework regions and CDRs, specifically from FR1 through FR4 or CDR1 through CDR3, of any one of the sequences of monoclonal antibodies 4C1, 2A4 or 6C7, as shown in Table 7 or Table 8. A further embodiment of the disclosure is antibodies comprising a sequence comprising the contiguous sequence spanning the framework regions and CDRs, specifically from FR1 through FR4 or CDR1 through CDR3, of any one of the sequences of monoclonal antibodies 4C1, 2A4 or 6C7 as shown in Table 7 and Table 8. In some embodiments, the antibody is a fully human monoclonal antibody.

One embodiment provides an antibody, or antigen-binding portion thereof, wherein the antibody, or antigen-binding portion thereof, comprises a sequence comprising SEQ ID NO: 2, SEQ ID NO: 4, SEQ ID NO: 6, SEQ ID NO: 8, SEQ ID NO: 10 or SEQ ID NO: 12.

In another embodiment, an antibody of the disclosure, or antigen-binding portion thereof, comprises a heavy chain sequence comprising the sequence of SEQ ID NO: 2. In other embodiments, an antibody of the disclosure, or antigen-binding portion thereof, further comprises a light chain sequence comprising the sequence of SEQ ID NO: 4. In some embodiments, the antibody is a fully human monoclonal antibody.

In another embodiment, an antibody of the disclosure, or antigen-binding portion thereof, comprises a heavy chain variable domain having at least 90% identity to the amino acid of SEQ ID NO: 2 and comprises a light chain variable domain having at least 90% identity to the amino acid sequence of SEQ ID NO: 4.

Another embodiment provides an antibody, or antigen-binding portion thereof, wherein the antibody, or antigen-binding portion thereof, comprises a heavy chain sequence comprising the sequence of SEQ ID NO: 6. In one embodiment, the antibody, or antigen-binding portion thereof, further comprises a light chain sequence comprising the sequence of SEQ ID NO: 8. In some embodiments, the antibody is a fully human monoclonal antibody.

In another embodiment, and antibody of the disclosure, or antigen-binding portion thereof, comprises a heavy chain variable domain having at least 90% identity to the amino acid of SEQ ID NO: 6 and comprises a light chain variable domain having at least 90% identity to the amino acid sequence of SEQ ID NO: 8.

In another embodiment, an antibody of the disclosure, or antigen-binding portion thereof, comprises a heavy chain sequence comprising the sequence of SEQ ID NO: 10. In another embodiment, the antibody, or antigen-binding portion thereof, further comprises a light chain sequence comprising the sequence of SEQ ID NO: 12. In some embodiments, the antibody is a fully human monoclonal antibody.

In another embodiment, an antibody of the disclosure, or antigen-binding portion thereof, comprises a heavy chain variable domain having at least 90% identity to the amino acid of SEQ ID NO: 10 and comprises a light chain variable domain having at least 90% identity to the amino acid sequence of SEQ ID NO: 12.

In other embodiments, an antibody of the disclosure comprises variants or derivatives of the CDRs disclosed herein, the contiguous sequences spanning the framework regions and CDRs (specifically from FR1 through FR4 or CDR1 through CDR3), the light or heavy chain sequences disclosed herein, or the antibodies disclosed herein. Variants include antibodies comprising sequences which have as many as twenty, sixteen, ten, nine or fewer, e.g. one, two, three, four, five or six amino acid additions, substitutions, deletions, and/or insertions in any one or more of the CDR1, CDR2 or CDR3s as shown in Table 7 or Table 8, the contiguous sequences spanning the framework regions and CDRs (specifically from FR1 through FR4 or CDR1 through CDR3) as shown in Table 7 or Table 8, the light or heavy chain sequences disclosed herein, or with the monoclonal antibodies disclosed herein. Variants include antibodies comprising sequences which have one, two or three, amino acid additions, substitutions, deletions, and/or insertions in any one or more of the CDR1, CDR2 or CDR3s as shown in Table 7 or Table 8, the contiguous sequences spanning the framework regions and CDRs (specifically from FR1 through FR4 or CDR1 through CDR3) as shown in Table 7 or Table 8, the light or heavy chain sequences disclosed herein, or with the monoclonal antibodies disclosed herein. Variants include antibodies comprising sequences which have at least about 60, 70, 80, 85, 90, 95, 98 or about 99% amino acid sequence identity with any of the CDR1, CDR2 or CDR3s as shown in Table 7 or Table 8, the contiguous sequences spanning the framework regions and CDRs (specifically from FR1 through FR4 or CDR1 through CDR3) as shown in Table 7 or Table 8, the light or heavy chain sequences disclosed herein, or with the monoclonal antibodies disclosed herein. The percent identity of two amino acid sequences can be determined by any method known to one skilled in the art, including, but not limited to, pairwise protein alignment. In one embodiment, variants comprise changes in the CDR sequences or light or heavy chain sequences disclosed herein that are naturally occurring or are introduced by in vitro engineering of native sequences using recombinant DNA techniques or mutagenesis techniques. Naturally occurring variants include those which are generated in vivo in the corresponding germline nucleotide sequences during the generation of an antibody to a foreign antigen. In one embodiment, the derivative may be a heteroantibody, that is an antibody in which two or more antibodies are linked together. Derivatives include antibodies which have been chemically modified. Examples include covalent attachment of one or more polymers, such as water-soluble polymers, N-linked, or O-linked carbohydrates, sugars, phosphates, and/or other such molecules. The derivatives are modified in a manner that is different from naturally occurring or starting antibody, either in the type or location of the molecules attached. Derivatives further include deletion of one or more chemical groups which are naturally present on the antibody.

In some embodiments of the disclosure, the antibody comprises a sequence comprising SEQ ID NO: 6. In some embodiments of the disclosure, the antibody comprises a sequence comprising SEQ ID NO: 6, wherein SEQ ID NO: 6 comprises any one of the unique combinations of germline and non-germline residues indicated by each row of Table 7. In some embodiments of the disclosure, the antibody comprises a sequence comprising SEQ ID NO: 6, wherein SEQ ID NO: 6 comprises any one, any two, any three, any four or all four of the germline residues as indicated in Table 7.

In some embodiments of the disclosure, the antibody comprises a sequence comprising SEQ ID NO: 8. In some embodiments of the disclosure, the antibody comprises a sequence comprising SEQ ID NO: 8, wherein SEQ ID NO: 8 comprises any one of the unique combinations of germline and non-germline residues indicated by each row of Table 8. In some embodiments of the disclosure, the antibody comprises a sequence comprising SEQ ID NO: 8, wherein SEQ ID NO: 8 comprises any one, any two, or all two of the germline residues as indicated in Table 8.

In some embodiments of the disclosure, the antibody comprises a sequence comprising SEQ ID NO: 12. In some embodiments of the disclosure, the antibody comprises a sequence comprising SEQ ID NO: 12, wherein SEQ ID NO.: 12 comprises any one of the unique combinations of germline and non-germline residues indicated by each row of Table 9. In some embodiments of the disclosure, the antibody comprises a sequence comprising SEQ ID NO: 12, wherein SEQ ID NO: 12 comprises any one, any two, any three, or all three of the germline residues as indicated in Table 9.

Antibodies of the disclosure may also inhibit tumour growth, cell adhesion, motility, invasion, and/or cellular metastasis and, in addition, the targeted binding agents are useful for reducing tumour growth and angiogenesis. Mechanisms by which this can be achieved can include, and are not limited to, inhibiting CXCR4 activity and/or blocking SDF-1 binding to the CXCR4 receptor.

Further embodiments of the disclosure relate to antibodies of the disclosure that inhibit angiogenesis. In one embodiment, an antibody of the disclosure inhibits at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% human vessel formation compared to a control. In one example, 6C7 inhibits human vessel formation by at least 70%. The following provides additional description of functional and structural characteristics of the anti-CXCR4 antibodies (and antigen binding fragments) of the disclosure. The disclosure contemplates that antibodies of the disclosure specifically bind to human CXCR4 and possess any one or more (or any two or more, three or more, four or more, five or more, six or more, etc.) of the structural and/or functional characteristics of CXCR4 antibodies described herein. Throughout this section of the specification, the term antibody or antibodies is used for convenience to refer to CXCR4 antibodies or antigen binding fragments, and thus all descriptions of functional and structural characteristics of antibodies apply, unless contexts indicates otherwise, to antigen binding fragments of the disclosure.

In certain embodiments, the anti-CXCR4 antibodies are isolated and/or purified and/or pyrogen free antibodies. The term “purified” as used herein, refers to other molecules, e.g. polypeptide, nucleic acid molecule that have been identified and separated and/or recovered from a component of its natural environment. Thus, in one embodiment the antibodies of the disclosure are purified antibodies wherein they have been separated from one or more components of their natural environment. The term “isolated antibody” as used herein refers to an antibody which is substantially free of other antibody molecules having different antigenic specificities (e.g., an isolated antibody that specifically binds to CXCR4 is substantially free of antibodies that specifically bind antigens other than CXCR4; however a bi- or multi-specific antibody molecule is an isolated antibody when substantially free of other antibody molecules). Thus, in one embodiment, the antibodies of the disclosure are isolated antibodies wherein they have been separated from antibodies with a different specificity. Typically an isolated antibody is a monoclonal antibody. An isolated antibody that specifically binds to an epitope, isoform or variant of human CXCR4 may, however, have cross-reactivity to other related antigens, e.g., from other species (e.g., CXCR4 species homologs). For example, an antibody of the disclosure may specifically bind to human CXCR4 and specifically bind to cynomolgous CXCR4. Moreover, an isolated antibody of the disclosure may be substantially free of one or more other cellular materials and/or chemicals and is herein referred to as an isolated and purified antibody. In one embodiment of the disclosure, a combination of “isolated” monoclonal antibodies relates to antibodies having different specificities and being combined in a well defined composition. Methods of production and purification/isolation are described below in more detail. This definition similarly applies to antigen binding fragments. In certain embodiments, an antibody of the disclosure may be a humanized antibody, a chimeric antibody or a human antibody.

The isolated antibodies or antigen binding fragments of the present disclosure comprise antibody amino acid sequences disclosed herein encoded by any suitable polynucleotide, or any isolated or formulated antibody. In one embodiment, the anti-CXCR4 antibody binds human CXCR4 and, thereby partially or substantially alters at least one biological activity of CXCR4 Antibody-producing cells encoding antibodies have been placed with the American Type Culture Collection (ATCC, 10801 University Blvd., Manassas, Va. 20110-2209), as described above. These deposits will be maintained under the terms of the Budapest Treaty on the International Recognition of the Deposit of Microorganisms for the Purposes of Patent Procedure. Examples of anti-CXCR4 antibodies of the disclosure are antibodies produced by such cells. Further examples include antibodies or antigen binding fragments that bind the same epitope as any of the deposited antibodies.

The anti-CXCR4 antibodies of the disclosure specifically bind at least one specified epitope specific to the CXCR4 protein, peptide, subunit, fragment, portion or any combination thereof and do not specifically bind to other polypeptides. The at least one epitope can comprise at least one antibody binding region that comprises at least one portion of the CXCR4 protein. The term “epitope” as used herein refers to a protein determinant capable of binding to an antibody. Epitopes usually consist of chemically active surface groupings of molecules such as amino acids or sugar side chains and usually have specific three dimensional structural characteristics, as well as specific charge characteristics. Conformational and non-conformational epitopes are distinguished in that the binding to the former but not the latter is lost in the presence of denaturing solvents.

In certain embodiments, an anti-CXCR4 antibody or antigen binding fragment of the present disclosure binds to the second loop (the second extracellular loop) of human CXCR4. Thus, in certain embodiments, the epitope to which the antibody binds is within the second loop of CXCR4. The second loop of human CXCR4 comprises amino acids 177-200 of human CXCR4. This region of CXCR4 is shorter in humans than in mice.

The amino acid sequence of human CXCR4 is set forth below with residues 177-200 underlined and bolded:

(human CXCR4; SEQ ID NO: 25)
MEGISIYTSDNYTEEMGSGDYDSMKEPCFREENANFNKIFLPTIYSI
IFLTGIVGNGLVILVMGYQKKLRSMTDKYRLHLSVADLLFVITLPFW
AVDAVANWYFGNFLCKAVHVIYTVNLYSSVLILAFISLDRYLAIVHA
TNSQRPRKLLAEKVVYVGVWIPALLLTIPDFIFANVSEADDRYICDR
FYPNDLWVVVFQFQHIMVGLILPGIVILSCYCIIISKLSHSKGHQKR
KALKTTVILILAFFACWLPYYIGISIDSFILLEIIKQGCEFENTVHK
WISITEALAFFHCCLNPILYAFLGAKFKTSAQHALTSVSRGSSLKIL
SKGKRGGHSSVSTESESSSFHSS.

The amino acid sequence of mouse CXCR4 is set forth below with the corresponding residues of the second loop underlined and bolded:

(mouse CXCR4; SEQ ID NO: 26)
MEPISVSIYTSDNYSEEVGSGDYDSNKEPCFRDENVHFNRIFLPTIY
FIIFLTGIVGNGLVILVMGYQKKLRSMTDKYRLHLSVADLLFVITLP
FWAVDAMADWYFGKFLCKAVHIIYTVNLYSSVLILAFISLDRYLAIV
HATNSQRPRKLLAEKAVYVGVWIPALLLTIPDFIFADVSQGDISQGD
DRYICDRLYPDSLWMVVFQFQHIMVGLILPGIVILSCYCIIISKLSH
SKGHQKRKALKTTVILILAFFACWLPYYVGISIDSFILLGVIKQGCD
FESIVHKWISITEALAFFHCCLNPILYAFLGAKFKSSAQHALNSMSR
GSSLKILSKGKRGGHSSVSTESESSSFHSS.

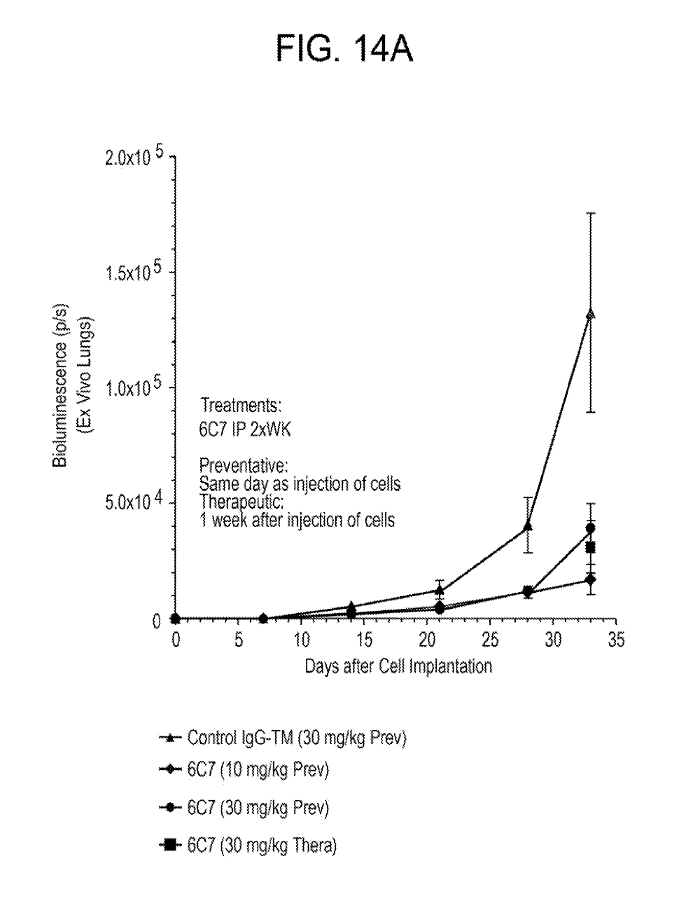

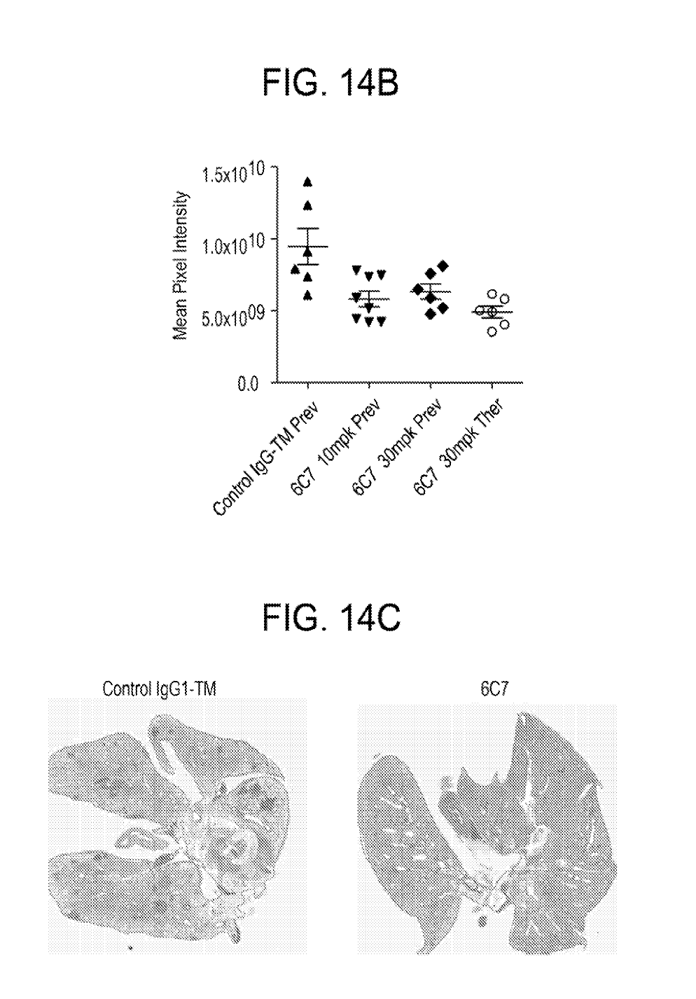

The structure of CXCR4 protein is known in the art. Exemplary publications include Chabot et al., (1999) Journal of Virology 73(8): 6598-6609 and Roland et al. (2003) Blood 101: 399-406. FIG. 14 also provides a representation of the structure of CXCR4.

In certain embodiments, an anti-CXCR4 antibody or antigen binding fragment of the present disclosure binds specifically bind to human CXCR4 and also binds specifically bind to CXCR4 from one or more of mouse, rat, and cynomolgous monkey. In other embodiments, an anti-CXCR4 antibody or antigen binding fragment of the disclosure binds specifically bind to human CXCR4 but does not bind specifically bind to mouse and/or rat CXCR4. In other embodiments, an anti-CXCR4 antibody or antigen binding fragment of the disclosure binds specifically bind to human CXCR4 and also binds specifically to cynomolgous CXCR4.

In certain embodiments an antibody of the disclosure specifically binds to CXCR4, and has one or more (one, two, three, four, five, six, seven, eight or nine) of the following properties selected from the group consisting of:
binds human CXCR4 with a K_{D} of less than 2.5 nanomolar (nM) when measured by FACS binding kinexa analysis;
cross-reacts with cynomolgus monkey CXCR4 with a K_{D} of less than 1 nM when measured by FACS binding kinexa analysis;
does not bind significantly to CXCR3 or CCR4;
inhibits SDF-1 binding to CXCR4;
inhibits SDF-1 pMAPK phosphorylation;
inhibits SDF-1 induced Jurkat chemotaxis with an IC50 of less than 0.5 nM;
inhibits SDF-1 HUVEC migration at an IC50 concentration of below 10 nM;
induces apoptosis in Ramos cells; and
causes no more than a 60% reduction of B-cell counts when added to a peripheral blood leukocyte cell preparation at a concentration of 10 ug/ml over a period of 16-18 hours.

In another embodiment, an antibody of the disclosure has any one or more of the foregoing characteristics, and also comprises a VH and/or VL domain comprising an amino acid sequence of a VH or VL domain of any of the exemplary antibodies provided herein. In another embodiment, an antibody of the disclosure has any one or more of the foregoing characteristics, and also comprises a heavy chain comprising a CDR1, CDR2, and CDR3 of any of the exemplary antibodies provided herein and/or a light chain comprising a CDR1, CDR2, and CDR3 of any of the exemplary antibodies provided herein.

### (iii) Nucleic Acid Molecules and Host Cells

The disclosure also provides nucleic acid molecules encoding any of the antibodies of the disclosure. In certain embodiments, the disclosure provides a nucleic acid molecule encoding the light chain and/or the heavy chain of an antibody of the disclosure. In another embodiment, the disclosure provides a nucleic acid molecule encoding the light chain and/or the heavy chain of a fully human monoclonal antibody. In another embodiment, the disclosure provides a nucleic acid molecule encoding the light chain and/or the heavy chain of any one of the fully human monoclonal antibodies described herein including 6C7, 2A4, and 4C1. The disclosure also encompasses polynucleotides that hybridize under stringent or lower stringency hybridization conditions, as defined herein, to polynucleotides that encode any of the targeted binding agents or antibodies described herein.

In another embodiment of the disclosure there is provided a vector comprising a nucleic acid molecule or molecules as described hereinabove, wherein the vector encodes an antibody (or antigen binding fragment) of the disclosure. In one embodiment of the disclosure there is provided a vector comprising a nucleic acid molecule or molecules as described hereinabove, wherein the vector encodes a light chain and/or a heavy chain of an antibody as defined hereinabove. In one embodiment, the vector comprises a nucleic acid molecule encoding the light chain and/or the heavy chain of a fully human monoclonal antibody. In one embodiment, the vector comprises a nucleic acid molecule encoding the light chain or the heavy chain of any one of the human monoclonal antibodies described herein including 6C7, 2A4, and 4C1. In another embodiment, the vector comprises a nucleic acid molecule encoding the light chain and the heavy chain of any one of the human monoclonal antibodies described herein including 6C7, 2A4, and 4C1.

In a further embodiment there is provided a host cell transformed with any of the nucleic acid molecules as described hereinabove. In another embodiment of the disclosure there is provided a host cell comprising the vector comprising the nucleic acid molecule as described hereinabove. In one embodiment, the host cell may comprise more than one vector.

### (iv) Production of Anti-CXCR4 Antibodies

The following describes exemplary techniques for the production of the antibodies useful in the present disclosure. Some of these techniques are described further in the Examples section. The CXCR4 antigen to be used for production of antibodies may be human CXCR4 or an antigenic fragment thereof. Alternatively, cells expressing CXCR4 at their cell surface or membranes prepared from such cells can be used to generate antibodies. The nucleotide and amino acid sequences of CXCR4, such as human CXCR4, are readily available. CXCR4 can be produced recombinantly in an isolated form from, bacterial or eukaryotic cells using standard recombinant DNA methodology. CXCR4 can be expressed as a tagged (e.g., epitope tag) or other fusion protein to facilitate isolation as well as identification in various assays. Antibodies or binding proteins that bind to various tags and fusion sequences are available as elaborated below. Other forms of CXCR4 useful for generating antibodies will be apparent to those skilled in the art.

(a) Tags

Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Engineering, 3(6):547-553 (1990)). The FLAG-peptide (Hopp et al., BioTechnology, 6:1204-1210 (1988)) is recognized by an anti-FLAG M2 monoclonal antibody (Eastman Kodak Co., New Haven, Conn.). Purification of a protein containing the FLAG peptide can be performed by immunoaffinity chromatography using an affinity matrix comprising the anti-FLAG M2 monoclonal antibody covalently attached to agarose (Eastman Kodak Co., New Haven, Conn.). Other tag polypeptides include the KT3 epitope peptide (Martin et al., Science, 255:192-194 (1992)); an α-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

(b) Monoclonal Antibodies

Monoclonal antibodies can be prepared using a wide variety of techniques known in the art including the use of hybridoma (Kohler et al., Nature, 256:495 (1975); Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981), recombinant, and phage display technologies, or a combination thereof. The term “monoclonal antibody” as used herein refers to an antibody obtained from a population of substantially homogeneous or isolated antibodies, e.g., the individual antibodies comprising the population are identical except for possible naturally occurring mutations that may be present in minor amounts. Monoclonal antibodies are highly specific, being directed against a single antigenic site or multiple antigenic sites in the case of multispecific engineered antibodies. Furthermore, in contrast to polyclonal antibody preparations which include different antibodies directed against different determinants (epitopes), each monoclonal antibody is directed against the same determinant on the antigen. In addition to their specificity, the monoclonal antibodies are advantageous in that they may be synthesized uncontaminated by other antibodies. The modifier “monoclonal” is not to be construed as requiring production of the antibody by any particular method. Following is a description of representative methods for producing monoclonal antibodies which is not intended to be limiting and may be used to produce, for example, monoclonal mammalian, chimeric, humanized, human, domain, diabodies, vaccibodies, linear and multispecific antibodies.

A. Hybridoma Techniques

Methods for producing and screening for specific antibodies using hybridoma technology are routine and well known in the art. In the hybridoma method, mice or other appropriate host animals, such as hamster, are immunized to elicit lymphocytes that produce or are capable of producing antibodies that will specifically bind to the antigen used for immunization. Alternatively, lymphocytes may be immunized in vitro, as is sometimes done when using hybridoma technology to produce human monoclonal antibodies. After immunization (in vivo or in vitro), lymphocytes are isolated and then fused with a myeloma cell line using a suitable fusing agent or fusion partner, such as polyethylene glycol, to form a hybridoma cell (Goding, Monoclonal Antibodies: Principles and Practice, pp. 59-103 (Academic Press, 1986)). In certain embodiments, the selected myeloma cells are those that fuse efficiently, support stable high-level production of antibody by the selected antibody-producing cells, and are sensitive to a selective medium that selects against the unfused parental cells. In one aspect, the myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Once hybridoma cells that produce antibodies of the desired specificity, affinity, and/or activity are identified, the clones may be subcloned by limiting dilution procedures and grown by standard methods (Goding, Supra). Suitable culture media for this purpose include, for example, D-MEM or RPMI-1640 medium. In addition, the hybridoma cells may be grown in vivo as ascites tumors in an animal e.g., by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, affinity tags, hydroxylapatite chromatography, gel electrophoresis, dialysis, etc. Exemplary purification methods are described in more detail below.

B. Recombinant DNA Techniques

Methods for producing and screening for specific antibodies using recombinant DNA technology are routine and well known in the art (e.g. U.S. Pat. No. 4,816,567). DNA encoding the monoclonal antibodies may be readily isolated and/or sequenced using conventional procedures (e.g., by using oligonucleotide probes that are capable of binding specifically to genes encoding the heavy and light chains of murine antibodies). Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Pluckthun, Immunol. Revs., 130:151-188 (1992). As described below for antibodies generated by phage display and humanization of antibodies, DNA or genetic material for recombinant antibodies can be obtained from source(s) other than hybridomas to generate antibodies of the disclosure.

Recombinant expression of an antibody or variant thereof generally requires construction of an expression vector containing a polynucleotide that encodes the antibody. The disclosure, thus, provides replicable vectors comprising a nucleotide sequence encoding an antibody molecule, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., U.S. Pat. Nos. 5,981,216; 5,591,639; 5,658,759 and 5,122,464) and the variable domain of the antibody may be cloned into such a vector for expression of the entire heavy, the entire light chain, or both the entire heavy and light chains.

Once the expression vector is transferred to a host cell by conventional techniques, the transfected cells are then cultured by conventional techniques to produce an antibody. Thus, the disclosure includes host cells containing a polynucleotide encoding an antibody of the disclosure or fragments thereof, or a heavy or light chain thereof, or portion thereof, or a single-chain antibody of the disclosure, operably linked to a heterologous promoter. In certain embodiments for the expression of double-chained antibodies, vectors encoding both the heavy and light chains may be co-expressed in the host cell for expression of the entire immunoglobulin molecule, as detailed below.

Mammalian cell lines available as hosts for expression of recombinant antibodies are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines. Different host cells have characteristic and specific mechanisms for the post-translational processing and modification of proteins and gene products. Appropriate cell lines or host systems can be chosen to ensure the correct modification and processing of the antibody or portion thereof expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any functional immunoglobulin chains), SP20, CRL7O3O and HsS78Bst cells. In one embodiment, human cell lines developed by immortalizing human lymphocytes can be used to recombinantly produce monoclonal antibodies. In one embodiment, the human cell line PER.C6. (Crucell, Netherlands) can be used to recombinantly produce monoclonal antibodies.

Additional cell lines which may be used as hosts for expression of recombinant antibodies include, but are not limited to, insect cells (e.g. Sf21/5f9, Trichoplusia ni Bti-Tn5b1-4) or yeast cells (e.g. S. cerevisiae, Pichia, U.S. Pat. No. 7,326,681; etc), plants cells (US20080066200); and chicken cells (WO2008142124).

In certain embodiments, antibodies of the disclosure are expressed in a cell line with stable expression of the antibody. Stable expression can be used for long-term, high-yield production of recombinant proteins. For example, cell lines which stably express the antibody molecule may be generated. Host cells can be transformed with an appropriately engineered vector comprising expression control elements (e.g., promoter, enhancer, transcription terminators, polyadenylation sites, etc.), and a selectable marker gene. Following the introduction of the foreign DNA, cells may be allowed to grow for 1-2 days in an enriched media, and then are switched to a selective media. The selectable marker in the recombinant plasmid confers resistance to the selection and allows cells that stably integrated the plasmid into their chromosomes to grow and form foci which in turn can be cloned and expanded into cell lines. Methods for producing stable cell lines with a high yield are well known in the art and reagents are generally available commercially.

In certain embodiments, antibodies of the disclosure are expressed in a cell line with transient expression of the antibody. Transient transfection is a process in which the nucleic acid introduced into a cell does not integrate into the genome or chromosomal DNA of that cell. It is in fact maintained as an extrachromosomal element, e.g. as an episome, in the cell. Transcription processes of the nucleic acid of the episome are not affected and a protein encoded by the nucleic acid of the episome is produced.

The cell line, either stable or transiently transfected, is maintained in cell culture medium and conditions well known in the art resulting in the expression and production of monoclonal antibodies. In certain embodiments, the mammalian cell culture media is based on commercially available media formulations, including, for example, DMEM or Ham's F12. In other embodiments, the cell culture media is modified to support increases in both cell growth and biologic protein expression. As used herein, the terms “cell culture medium,” “culture medium,” and “medium formulation” refer to a nutritive solution for the maintenance, growth, propagation, or expansion of cells in an artificial in vitro environment outside of a multicellular organism or tissue. Cell culture medium may be optimized for a specific cell culture use, including, for example, cell culture growth medium which is formulated to promote cellular growth, or cell culture production medium which is formulated to promote recombinant protein production. The terms nutrient, ingredient, and component are used interchangeably herein to refer to the constituents that make up a cell culture medium.

In one embodiment, the cell lines are maintained using a fed batch method. As used herein, “fed batch method,” refers to a method by which a fed batch cell culture is supplied with additional nutrients after first being incubated with a basal medium. For example, a fed batch method may comprise adding supplemental media according to a determined feeding schedule within a given time period. Thus, a “fed batch cell culture” refers to a cell culture wherein the cells, typically mammalian, and culture medium are supplied to the culturing vessel initially and additional culture nutrients are fed, continuously or in discrete increments, to the culture during culturing, with or without periodic cell and/or product harvest before termination of culture.

The cell culture medium used and the nutrients contained therein are known to one of skill in the art. In one embodiment, the cell culture medium comprises a basal medium and at least one hydrolysate, e.g., soy-based, hydrolysate, a yeast-based hydrolysate, or a combination of the two types of hydrolysates resulting in a modified basal medium. In another embodiment, the additional nutrients may include only a basal medium, such as a concentrated basal medium, or may include only hydrolysates, or concentrated hydrolysates. Suitable basal media include, but are not limited to Dulbecco's Modified Eagle's Medium (DMEM), DME/F12, Minimal Essential Medium (MEM), Basal Medium Eagle (BME), RPMI 1640, F-10, F-12, α-Minimal Essential Medium (α-MEM), Glasgow's Minimal Essential Medium (G-MEM), PF CHO (see, e.g., CHO protein free medium (Sigma) or EX-CELL™ 325 PF CHO Serum-Free Medium for CHO Cells Protein-Free (SAFC Bioscience), and Iscove's Modified Dulbecco's Medium. Other examples of basal media which may be used in the disclosure include BME Basal Medium (Gibco-Invitrogen; see also Eagle, H (1965) Proc. Soc. Exp. Biol. Med. 89, 36); Dulbecco's Modified Eagle Medium (DMEM, powder) (Gibco-Invitrogen (#31600); see also Dulbecco and Freeman (1959) Virology 8, 396; Smith et al. (1960) Virology 12, 185. Tissue Culture Standards Committee, In Vitro 6:2, 93); CMRL 1066 Medium (Gibco-Invitrogen (#11530); see also Parker R. C. et al (1957) Special Publications, N.Y. Academy of Sciences, 5, 303).

In certain embodiments, the basal medium may be serum-free, meaning that the medium contains no serum (e.g., fetal bovine serum (FBS), horse serum, goat serum, or any other animal-derived serum known to one skilled in the art) or animal protein free media or chemically defined media.

The basal medium may be modified in order to remove certain non-nutritional components found in standard basal medium, such as various inorganic and organic buffers, surfactant(s), and sodium chloride. Removing such components from basal cell medium allows an increased concentration of the remaining nutritional components, and may improve overall cell growth and protein expression. In addition, omitted components may be added back into the cell culture medium containing the modified basal cell medium according to the requirements of the cell culture conditions. In certain embodiments, the cell culture medium contains a modified basal cell medium, and at least one of the following nutrients, an iron source, a recombinant growth factor; a buffer; a surfactant; an osmolarity regulator; an energy source; and non-animal hydrolysates. In addition, the modified basal cell medium may optionally contain amino acids, vitamins, or a combination of both amino acids and vitamins. In another embodiment, the modified basal medium further contains glutamine, e.g., L-glutamine, and/or methotrexate.

In certain embodiments, antibody production is conducted in large quantity by a bioreactor process using fed-batch, batch, perfusion or continuous feed bioreactor methods known in the art. Large-scale bioreactors have at least 1000 liters of capacity, preferably about 1,000 to 100,000 liters of capacity. These bioreactors may use agitator impellers to distribute oxygen and nutrients. Small scale bioreactors refers generally to cell culturing in no more than approximately 100 liters in volumetric capacity, and can range from about 1 liter to about 100 liters. Alternatively, single-use bioreactors (SUB) may be used for either large-scale or small scale culturing.

Temperature, pH, agitation, aeration and inoculum density will vary depending upon the host cells used and the recombinant protein to be expressed. For example, a recombinant protein cell culture may be maintained at a temperature between 30 and 45 degrees Celsius. The pH of the culture medium may be monitored during the culture process such that the pH stays at an optimum level, which may be for certain host cells, within a pH range of 6.0 to 8.0. An impellor driven mixing may be used for such culture methods for agitation. The rotational speed of the impellor may be approximately 50 to 200 cm/sec tip speed, but other airlift or other mixing/aeration systems known in the art may be used, depending on the type of host cell being cultured. Sufficient aeration is provided to maintain a dissolved oxygen concentration of approximately 20% to 80% air saturation in the culture, again, depending upon the selected host cell being cultured. Alternatively, a bioreactor may sparge air or oxygen directly into the culture medium. Other methods of oxygen supply exist, including bubble-free aeration systems employing hollow fiber membrane aerators.

C. Phage Display Techniques

In another embodiment, monoclonal antibodies or antigen binding fragments can be isolated from antibody phage libraries generated using the techniques described in, for example, McCafferty et al., Nature, 348:552-554 (1990). Clackson et al., Nature, 352:624-628 (1991) and Marks et al., J. Mol. Biol., 222:581-597 (1991). In such methods, antibodies of the disclosure can be isolated by screening of a recombinant combinatorial antibody library, preferably a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SURFZAP™ phage display kit, catalog no. 240612), examples of methods and reagents particularly amenable for use in generating and screening antibody display libraries can be found in, for example, U.S. Pat. No. 6,248,516; U.S. Pat. Nos. 6,545,142; 6,291,158; 6,291,1591; 6,291,160; 6,291,161; 6,680,192; 5,969,108; 6,172,197; 6,806,079; 5,885,793; 6,521,404; 6,544,731; 6,555,313; 6,593,081; 6,582,915; 7,195,866. Thus, these techniques are viable alternatives to traditional monoclonal antibody hybridoma techniques for generation and isolation of monoclonal antibodies.

In phage display methods, functional antibody domains are displayed on the surface of phage particles which carry the polynucleotide sequences encoding them. In a particular embodiment, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein.

As described in the above references, after phage selection, the antibody coding regions from the phage can be isolated and used to generate whole antibodies, including human antibodies, humanized antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Better et al., Science 240:1041-1043 (1988).

Examples of techniques which can be used to produce single-chain Fvs and antibodies include those described in U.S. Pat. Nos. 4,946,778 and 5,258,498. Thus, techniques described above and those well known in the art can be used to generate recombinant antibodies wherein the binding domain, e.g. ScFv, was isolated from a phage display library.

(c) Antibody Purification and Isolation

Once an antibody molecule has been produced by recombinant or hybridoma expression, it may be purified by any method known in the art for purification of an immunoglobulin molecule, for example, by chromatography (e.g., ion exchange, affinity, particularly by affinity for the specific antigens Protein A or Protein G, and sizing column chromatography), centrifugation, differential solubility, or by any other standard technique for the purification of proteins. Further, the antibodies of the present disclosure or fragments thereof may be fused to heterologous polypeptide sequences (referred to herein as “tags”) described above or otherwise known in the art to facilitate purification.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology, 10:163-167 (1992) describe a procedure for isolating antibodies which are secreted into the periplasmic space of E. coli. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, hydrophobic interaction chromatography, ion exchange chromatography, gel electrophoresis, dialysis, and/or affinity chromatography either alone or in combination with other purification steps. The suitability of protein A as an affinity ligand depends on the species and isotype of any immunoglobulin Fc domain that is present in the antibody and will be understood by one of skill in the art. The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH₃ domain, the Bakerbond ABX resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin, SEPHAROSE chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, and performed at low salt concentrations (e.g., from about 0-0.25 M salt).

Thus, in certain embodiments is provided antibodies of the disclosure that are substantially purified/isolated. In one embodiment, these isolated/purified recombinantly expressed antibodies may be administered to a patient to mediate a prophylactic or therapeutic effect. In another embodiment these isolated/purified antibodies may be used to diagnose a CXCR4 mediated disease.

(d) Humanized and Chimeric Antibodies

In certain embodiments, the antibodies of the disclosure are humanized antibodies, which are generated using methods well known in the art. Humanized antibodies are antibody molecules derived from a non-human species antibody (also referred to herein as a donor antibody) that bind the desired antigen having one or more complementarity determining regions (CDRs) from the non-human species and a framework region from a human immunoglobulin molecule (also referred to herein as an acceptor antibody). Often, framework residues in the human framework regions will be substituted with the corresponding residue from the CDR donor antibody to alter, preferably improve, antigen binding and/or reduce immunogenicity. These framework substitutions are identified by methods well known in the art, e.g., by modeling of the interactions of the CDR and framework residues to identify framework residues important for antigen binding and sequence comparison to identify unusual framework residues at particular positions. (See, e.g., Riechmann et al., Nature 332:323 (1988)). In practice, and in certain embodiments, humanized antibodies are typically human antibodies in which some hypervariable region residues and possibly some FR residues are substituted by residues from analogous sites in rodent antibodies. In alternative embodiments, the FR residues are fully human residues.

Humanization can be essentially performed following the method of Winter and co-workers (Jones et al., Nature, 321:522-525 (1986); Reichmann et al., Supra; Verhoeyen et al., Science, 239:1534-1536 (1988)), by substituting hypervariable region sequences for the corresponding sequences of a human antibody. Specifically, humanized antibodies may be prepared by methods well known in the art including CDR grafting approaches (see, e.g., U.S. Pat. No. 6,548,640), veneering or resurfacing (U.S. Pat. Nos. 5,639,641 and 6,797,492; Studnicka et al., Protein Engineering 7(6):805-814 (1994); Roguska. et al., PNAS 91:969-973 (1994)), chain shuffling strategies (see e.g., U.S. Pat. No. 5,565,332; Rader et al., Proc. Natl. Acad. Sci. USA (1998) 95:8910-8915), molecular modeling strategies (U.S. Pat. No. 5,639,641), and the like. These general approaches may be combined with standard mutagenesis and recombinant synthesis techniques to produce anti-CXCR4 antibodies with desired properties.

CDR grafting is performed by replacing one or more CDRs of an acceptor antibody (e.g., a human antibody) with one or more CDRs of a donor antibody (e.g., a non-human antibody). Acceptor antibodies may be selected based on similarity of framework residues between a candidate acceptor antibody and a donor antibody and may be further modified to introduce similar residues. Following CDR grafting, additional changes may be made in the donor and/or acceptor sequences to optimize antibody binding and functionality.

Grafting of abbreviated CDR regions is a related approach. Abbreviated CDR regions include the specificity-determining residues and adjacent amino acids, including those at positions 27d-34, 50-55 and 89-96 in the light chain, and at positions 31-35b, 50-58, and 95-101 in the heavy chain. See (Padlan et al. (1995) FASEB J. 9: 133-9). Grafting of specificity-determining residues (SDRs) is premised on the understanding that the binding specificity and affinity of an antibody combining site is determined by the most highly variable residues within each of the CDR regions. Analysis of the three-dimensional structures of antibody-antigen complexes, combined with analysis of the available amino acid sequence data was used to model sequence variability based on structural dissimilarity of amino acid residues that occur at each position within the CDR. Minimally immunogenic polypeptide sequences consisting of contact residues, which are referred to as SDRs, are identified and grafted onto human framework regions.

Veneering or resurfacing is based on the concept of reducing potentially immunogenic amino acid sequences in a rodent or other non-human antibody by resurfacing the solvent accessible exterior of the antibody with human amino acid sequences. Thus, veneered antibodies appear less foreign to human cells. A non-human antibody is veneered by (1) identifying exposed exterior framework region residues in the non-human antibody, which are different from those at the same positions in framework regions of a human antibody, and (2) replacing the identified residues with amino acids that typically occupy these same positions in human antibodies.

By definition, humanized antibodies are chimeric antibodies. Chimeric antibodies are antibodies in which a portion of the heavy and/or light chain is identical with or homologous to corresponding sequences in antibodies derived from a particular species or belonging to a particular antibody class or subclass, while another portion of the chain(s) is identical with or homologous to corresponding sequences in antibodies derived from another species or belonging to another antibody class or subclass, as well as fragments of such antibodies, so long as they exhibit the desired biological activity (e.g., Morrison et al., Proc. Natl. Acad. Sci. USA, 81:6851-6855 (1984)). Chimeric antibodies of interest herein include “primatized” antibodies comprising variable domain antigen-binding sequences derived from a nonhuman primate (e.g., Old World Monkey, such as baboon, rhesus or cynomolgus monkey) and human constant region sequences (U.S. Pat. No. 5,693,780).

(e) Human Antibodies

As an alternative to humanization, human antibodies can be generated using methods well known in the art. Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); U.S. Pat. No. 5,545,807; and WO 97/17852. In practice, the use of XENOMOUSE® strains of mice that have been engineered to contain up to but less than 1000 kb-sized germline configured fragments of the human heavy chain locus and kappa light chain locus. See Mendez et al. Nature Genetics 15:146-156 (1997) and Green and Jakobovits J. Exp. Med. 188:483-495 (1998). The XENOMOUSE® strains are available from Amgen, Inc. (Fremont, Calif.).

The production of the XENOMOUSE® strains of mice and antibodies produced in those mice is further discussed and delineated in U.S. Pat. Nos. 6,673,986; 7,049,426; 6,833,268; 6,162,963, 6,150,584, 6,114,598, 6,075,181, 6,657,103; 6,713,610 and 5,939,598; US Publication Nos. 2004/0010810; 2003/0229905; 2004/0093622; 2005/0054055; 2005/0076395; and 2006/0040363.

Essentially, XENOMOUSE® lines of mice are immunized with an antigen of interest (e.g. CXCR4), lymphatic cells (such as B-cells) are recovered from the hyper-immunized mice, and the recovered lymphocytes are fused with a myeloid-type cell line to prepare immortal hybridoma cell lines using techniques described above and well known in the art. These hybridoma cell lines are screened and selected to identify hybridoma cell lines that produced antibodies specific to the antigen of interest.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and usually a second constant region (preferably a gamma constant region) are formed into a construct for insertion into an animal. This approach is described in U.S. Pat. Nos. 5,545,807; 5,545,806; 5,625,825; 5,625,126; 5,633,425; 5,661,016; 5,770,429; 5,789,650; 5,814,318; 5,877,397; 5,874,299; 6,255,458; 5,591,669; 6,023,010; 5,612,205; 5,721,367; 5,789,215; 5,643,763; and 5,981,175.

Kirin has also demonstrated the generation of human antibodies from mice in which, through microcell fusion, large pieces of chromosomes, or entire chromosomes, have been introduced. See U.S. Pat. No. 6,632,976. Additionally, KM™ mice, which are the result of cross-breeding of Kirin's Tc mice with Medarex's minilocus (Humab) mice have been generated. These mice possess the human IgH transchromosome of the Kirin mice and the kappa chain transgene of the Genpharm mice (Ishida et al., Cloning Stem Cells, (2002) 4:91-102).

Human antibodies can also be derived by in vitro methods. Suitable examples include but are not limited to phage display (MedImmune (formerly CAT), Morphosys, Dyax, Biosite/Medarex, Xoma, Symphogen, Alexion (formerly Proliferon), Affimed) ribosome display (MedImmune (formerly CAT)), yeast display, and the like. The phage display technology (See e.g., U.S. Pat. No. 5,969,108) can be used to produce human antibodies or antigen binding fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antigen binding fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Immunoglobulin genes undergo various modifications during maturation of the immune response, including recombination between V, D and J gene segments, isotype switching, and hypermutation in the variable regions. Recombination and somatic hypermutation are the foundation for generation of antibody diversity and affinity maturation, but they can also generate sequence liabilities that may make commercial production of such immunoglobulins as therapeutic agents difficult or increase the immunogenicity risk of the antibody. In general, mutations in CDR regions are likely to contribute to improved affinity and function, while mutations in framework regions may increase the risk of immunogenicity. This risk can be reduced by reverting framework mutations to germline while ensuring that activity of the antibody is not adversely impacted. The diversification processes may also generate some structural liabilities or these structural liabilities may exist within germline sequences contributing to the heavy and light chain variable domains. Regardless of the source, it may be desirable to remove potential structural liabilities that may result in instability, aggregation, heterogeneity of product, or increased immunogenicity. Examples of undesirable liabilities include unpaired cysteines (which may lead to disulfide bond scrambling, or variable sulfhydryl adduct formation), N-linked glycosylation sites (resulting in heterogeneity of structure and activity), as well as deamidation (e.g. NG, NS), isomerization (DG), oxidation (exposed methionine), and hydrolysis (DP) sites.

Accordingly, in order to reduce the risk of immunogenicity and improve pharmaceutical properties of the antibodies of the disclosure, it may be desirable to revert a framework sequence to germline, revert a CDR to germline, and/or remove a structural liability.

(f) Antigen Binding Fragments

In certain embodiments, the present antibodies are antigen binding fragments or antibodies comprising these fragments. The antigen binding fragment comprises a portion of the full length antibody, which generally is the antigen binding or variable region thereof. Examples of antigen binding fragments include Fab, Fab′, F(ab′)₂, Fd and Fv fragments. Diabodies; linear antibodies (U.S. Pat. No. 5,641,870); single-chain antibody molecules; and multispecific antibodies are antibodies formed from these antigen binding fragments.

Traditionally, these fragments were derived via proteolytic digestion of intact antibodies using techniques well known in the art. However, these fragments can now be produced directly by recombinant host cells. Fab, Fv and scFv antigen binding fragments can all be expressed in and secreted from E. coli or other cell type, thus allowing the facile production of large amounts of these fragments. In one embodiment, the antigen binding fragments can be isolated from the antibody phage libraries discussed above. Alternatively, Fab′-SH fragments can also be directly recovered from E. coli and chemically coupled to form F(ab′)₂ fragments (Carter et al., Bio/Technology, 10:163-167 (1992)). According to another approach, F(ab′)₂ fragments can be isolated directly from recombinant host cell culture. Other techniques for the production of antigen binding fragments will be apparent to the skilled practitioner. In other embodiments, the antibody of choice is a single-chain Fv fragment (scFv). In certain embodiments, the antibody is not a Fab fragment. Fv and scFv are the only species with intact combining sites that are devoid of constant regions; thus, they are suitable for reduced nonspecific binding during in vivo use. scFv fusion proteins may be constructed to yield fusion of an effector protein at either the amino or the carboxy terminus of an scFv.

In certain embodiments, the present antibodies are domain antibodies, e.g., antibodies containing the small functional binding units of antibodies, corresponding to the variable regions of the heavy (V_{H}) or light (V_{L}) chains of human antibodies. Examples of domain antibodies include, but are not limited to, those available from Domantis that are specific to therapeutic targets (see, for example, WO04/058821; WO04/081026; WO04/003019; WO03/002609; U.S. Pat. Nos. 6,291,158; 6,582,915; 6,696,245; and 6,593,081). Commercially available libraries of domain antibodies can be used to identify anti-CXCR4 domain antibodies. In certain embodiments, anti-CXCR4 antibodies comprise a CXCR4 functional binding unit and an Fc gamma receptor functional binding unit.

In certain embodiments of the disclosure, the present antibodies are vaccibodies. Vaccibodies are dimeric polypeptides. Each monomer of a vaccibody consists of a scFv with specificity for a surface molecule on APC connected through a hinge region and a Cy3 domain to a second scFv. In other embodiments of the disclosure, vaccibodies containing as one of the scFv's an anti-CXCR4 antigen binding fragment may be used to juxtapose those cells to be destroyed and an effector cell that mediates ADCC. For example, see, Bogen et al., U.S. Patent Application Publication No. 2004/0253238.

In certain embodiments of the disclosure, the present antibodies are linear antibodies. Linear antibodies comprise a pair of tandem Fd segments (V_{H}-C_{H1}-V_{H}-C_{H1}) which form a pair of antigen-binding regions. Linear antibodies can be bispecific or monospecific. See, Zapata et al., Protein Eng., 8(10):1057-1062 (1995).

(g) Bispecific Antibodies

Bispecific antibodies are antibodies that have binding specificities for at least two different epitopes. Exemplary bispecific antibodies may bind to two different epitopes of the CXCR4 protein. Other such antibodies may combine a CXCR4 binding site with a binding site for another protein. Alternatively, an anti-CXCR4 arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the CXCR4-expressing cell. Bispecific antibodies may also be used to localize cytotoxic agents to cells which express CXCR4. These antibodies possess a CXCR4-binding arm and an arm which binds the cytotoxic agent (e.g. saporin, anti-interferon-α, vinca alkaloid, ricin A chain, methotrexate or radioactive isotope hapten). Bispecific antibodies can be prepared as full length antibodies or antigen binding fragments (e.g. F(ab′)₂ bispecific antibodies). Methods for making bispecific antibodies are known in the art. (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,819; 5,731,168; 4,676,980; 5,897,861; 5,660,827; 5,811,267; 5,849,877; 5,948,647; 5,959,084; 6,106,833; 6,143,873 and 4,676,980, WO 94/04690; and WO 92/20373.)

Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In one embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure may facilitate the separation of the desired bispecific compound from unwanted immunoglobulin chain combinations, as the presence of an immunoglobulin light chain in only one half of the bispecific molecule provides for a facile way of separation. For further details of generating bispecific antibodies see, for example, Suresh et al., Methods in Enzymology, 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (U.S. Pat. No. 5,897,861). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antigen binding fragments have also been described in the literature. For example, bispecific antibodies can be prepared using chemical linkage. Brennan et al., Science, 229: 81 (1985) describe a procedure wherein intact antibodies are proteolytically cleaved to generate F(ab)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antigen binding fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci USA, 90:6444-6448 (1993) has provided an alternative mechanism for making bispecific antigen binding fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antigen binding fragments by the use of single-chain Fv (sFv) dimers has also been reported. See Gruber et al., J. Immunol., 152:5368 (1994) and U.S. Pat. Nos. 5,591,828; 4,946,778; 5,455,030; and 5,869,620.

Antibodies with more than two valencies are contemplated. For example, trispecific antibodies can be prepared, Tutt et al. J. Immunol. 147: 60 (1991), and multispecific valencies U.S. Pat. No. 5,258,498.

(h) Other Amino Acid Sequence Modifications

In addition to the above described human, humanized and/or chimeric antibodies, the present disclosure also encompasses further modifications and, their variants and fragments thereof, of the anti-CXCR4 antibodies of the disclosure comprising one or more amino acid residues and/or polypeptide substitutions, additions and/or deletions in the variable light (V_{L}) domain and/or variable heavy (V_{H}) domain and/or Fc region and post translational modifications. Included in these modifications are antibody conjugates wherein an antibody has been covalently attached to a moiety. Moieties suitable for attachment to the antibodies include but are not limited to, proteins, peptides, drugs, labels, and cytotoxins. These changes to the antibodies may be made to alter or fine tune the characteristics (biochemical, binding and/or functional) of the antibodies as is appropriate for treatment and/or diagnosis of CXCR4 mediated diseases. Methods for forming conjugates, making amino acid and/or polypeptide changes and post-translational modifications are well known in the art, some of which are detailed below. The following description is not intended to be limiting, but instead a non-limiting description of some embodiments, more of which will be obvious to one of skill in the art. It is also understood that some of the following methods were used to develop the human, humanized and/or chimeric antibody sequences described above. Any combination of deletion, insertion, and substitution is made to arrive at the final construct, provided that the final construct possesses the desired characteristics.

Amino acid changes to the antibodies necessarily results in sequences that are less than 100% identical to the above identified antibody sequences or parent antibody sequence. In certain embodiments, in this context, the antibodies many have about 25% to about 95% sequence identity to the amino acid sequence of either the heavy or light chain variable domain of an anti-CXCR4 antibody as described herein. Thus, in one embodiment a modified antibody may have an amino acid sequence having at least 25%, 35%, 45%, 55%, 65%, 75%, 80%, 85%, 90%, or 95% amino acid sequence identity or similarity with the amino acid sequence of either the heavy or light chain variable domain of an anti-CXCR4 antibody as described herein. In another embodiment, an altered antibody may have an amino acid sequence having at least 25%, 35%, 45%, 55%, 65%, 75%, 80%, 85%, 90%, or 95% amino acid sequence identity or similarity with the amino acid sequence of the heavy or light chain CDR1, CDR2, or CDR3 of an anti-CXCR4 antibody as described herein. In another embodiment, an altered antibody may have an amino acid sequence having at least 25%, 35%, 45%, 55%, 65%, 75%, 80%, 85%, 90%, or 95% amino acid sequence identity or similarity with the amino acid sequence of the heavy or light chain FR1, FR2, FR3 or FR4 of an anti-CXCR4 antibody as described herein.

In certain embodiments, altered antibodies are generated by one or more amino acid alterations (e.g., substitutions, deletion and/or additions) introduced in one or more of the variable regions of the antibody. In another embodiment, the amino acid alterations are introduced in the framework regions. One or more alterations of framework region residues may result in an improvement in the binding affinity of the antibody for the antigen. This may be especially true when these changes are made to humanized antibodies wherein the framework region may be from a different species than the CDR regions. Examples of framework region residues to modify include those which non-covalently bind antigen directly (Amit et al., Science, 233:747-753 (1986)); interact with/effect the conformation of a CDR (Chothia et al., J. Mol. Biol., 196:901-917 (1987)); and/or participate in the V_{L}-V_{H} interface (U.S. Pat. Nos. 5,225,539 and 6,548,640). In one embodiment, from about one to about five framework residues may be altered. Sometimes, this may be sufficient to yield an antibody mutant suitable for use in preclinical trials, even where none of the hypervariable region residues have been altered. Normally, however, an altered antibody will comprise additional hypervariable region alteration(s). In certain embodiments, the hypervariable region residues may be changed randomly, especially where the starting binding affinity of an anti-CXCR4 antibody for the antigen from the second mammalian species is such that such randomly produced antibodies can be readily screened.

One useful procedure for generating altered antibodies is called “alanine scanning mutagenesis” (Cunningham and Wells, Science, 244:1081-1085 (1989)). In this method, one or more of the hypervariable region residue(s) are replaced by alanine or polyalanine residue(s) to alter the interaction of the amino acids with the CXCR4. Those hypervariable region residue(s) demonstrating functional sensitivity to the substitutions then are refined by introducing additional or other mutations at or for the sites of substitution. Thus, while the site for introducing an amino acid sequence variation is predetermined, the nature of the mutation per se need not be predetermined. The Ala-mutants produced this way are screened for their biological activity as described herein.

In certain embodiments the substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display (Hawkins et al., J. Mol. Biol., 254:889-896 (1992) and Lowman et al., Biochemistry, 30(45):10832-10837 (1991)). Briefly, several hypervariable region sites (e.g., 6-7 sites) are mutated to generate all possible amino acid substitutions at each site. The antibody mutants thus generated are displayed in a monovalent fashion from filamentous phage particles as fusions to the gene III product of M13 packaged within each particle. The phage-displayed mutants are then screened for their biological activity (e.g., binding affinity) as herein disclosed.

Mutations in antibody sequences may include substitutions, deletions, including internal deletions, additions, including additions yielding fusion proteins, or conservative substitutions of amino acid residues within and/or adjacent to the amino acid sequence, but that result in a “silent” change, in that the change produces a functionally equivalent anti-CXCR4 antibody. Conservative amino acid substitutions may be made on the basis of similarity in polarity, charge, solubility, hydrophobicity, hydrophilicity, and/or the amphipathic nature of the residues involved. For example, non-polar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. In addition, glycine and proline are residues that can influence chain orientation. Non-conservative substitutions will entail exchanging a member of one of these classes for a member of another class. Furthermore, if desired, non-classical amino acids or chemical amino acid analogs can be introduced as a substitution or addition into the antibody sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, Na-methyl amino acids, and amino acid analogs in general.

In another embodiment, any cysteine residue not involved in maintaining the proper conformation of the anti-CXCR4 antibody also may be substituted, generally with serine, to improve the oxidative stability of the molecule and prevent aberrant crosslinking Conversely, cysteine bond(s) may be added to the antibody to improve its stability (particularly where the antibody is an antigen binding fragment such as an Fv fragment).

In certain embodiments of the disclosure, an antibody can be modified to produce fusion proteins; i.e., the antibody, or a fragment thereof, fused to a heterologous protein, polypeptide or peptide. In certain embodiments, the protein fused to the portion of an antibody is an enzyme component of Antibody-Directed Enzyme Prodrug Therapy (ADEPT). Examples of other proteins or polypeptides that can be engineered as a fusion protein with an antibody include, but are not limited to toxins such as ricin, abrin, ribonuclease, DNase I, Staphylococcal enterotoxin-A, pokeweed anti-viral protein, gelonin, diphtherin toxin, Pseudomonas exotoxin, and Pseudomonas endotoxin. See, for example, Pastan et al., Cell, 47:641 (1986), and Goldenberg et al., Cancer Journal for Clinicians, 44:43 (1994). Enzymatically active toxins and fragments thereof which can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232.

Additional fusion proteins may be generated through the techniques of gene-shuffling, motif-shuffling, exon-shuffling, and/or codon-shuffling (collectively referred to as “DNA shuffling”). DNA shuffling may be employed to alter the characteristics of the antibody or fragments thereof (e.g., an antibody or a fragment thereof with higher affinities and lower dissociation rates). See, generally, U.S. Pat. Nos. 5,605,793; 5,811,238; 5,830,721; 5,834,252; and 5,837,458, and Patten et al., 1997, Curr. Opinion Biotechnol., 8:724-33; Harayama, 1998, Trends Biotechnol. 16(2):76-82; Hansson et al., 1999, J. Mol. Biol., 287:265-76; and Lorenzo and Blasco, 1998, Biotechniques 24(2):308-313. The antibody can further be a binding-domain immunoglobulin fusion protein as described in U.S. Publication 2003/0118592, and PCT Publication WO 02/056910.

A. Variant Fc Regions

It is known that variants of the Fc region (e.g., amino acid substitutions and/or additions and/or deletions) enhance or diminish effector function of the antibody (See e.g., U.S. Pat. Nos. 5,624,821; 5,885,573; 6,538,124; 7,317,091; 5,648,260; 6,538,124; WO 03/074679; WO 04/029207; WO 04/099249; WO 99/58572; US Publication No. 2006/0134105; 2004/0132101; 2006/0008883) and may alter the pharmacokinetic properties (e.g. half-life) of the antibody (see, U.S. Pat. Nos. 6,277,375 and 7,083,784). Thus, in certain embodiments, the anti-CXCR4 antibodies of the disclosure comprise an altered Fc region (also referred to herein as “variant Fc region”) in which one or more alterations have been made in the Fc region in order to change functional and/or pharmacokinetic properties of the antibodies. Such alterations may result in a decrease or increase of C1q binding and complement dependent cytotoxicity (CDC) or of FcγR binding, for IgG, and antibody-dependent cellular cytotoxicity (ADCC), or antibody dependent cell-mediated phagocytosis (ADCP). The present disclosure encompasses the antibodies described herein with variant Fc regions wherein changes have been made to fine tune the effector function, enhancing or diminishing, providing a desired effector function. Accordingly, in one embodiment of the disclosure, the anti-CXCR4 antibodies of the disclosure comprise a variant Fc region (i.e., Fc regions that have been altered as discussed below). Anti-CXCR4 antibodies of the disclosure comprising a variant Fc region are also referred to here as “Fc variant antibodies.” As used herein native refers to the unmodified parental sequence and the antibody comprising a native Fc region is herein referred to as a “native Fc antibody”. Fc variant antibodies can be generated by numerous methods well known to one skilled in the art. Non-limiting examples include, isolating antibody coding regions (e.g., from hybridoma) and making one or more desired substitutions in the Fc region of the isolated antibody coding region. Alternatively, the antigent-binding portion (e.g., variable regions) of an anti-CXCR4 antibody may be subcloned into a vector encoding a variant Fc region. In one embodiment, the variant Fc region exhibits a similar level of inducing effector function as compared to the native Fc region. In another embodiment, the variant Fc region exhibits a higher induction of effector function as compared to the native Fc. In another embodiment, the variant Fc region exhibits lower induction of effector function as compared to the native Fc. Some specific embodiments of variant Fc regions are detailed infra. Methods for measuring effector function are well known in the art.

The effector function of an antibody is modified through changes in the Fc region, including but not limited to, amino acid substitutions, amino acid additions, amino acid deletions and changes in post translational modifications to Fc amino acids (e.g. glycosylation). The methods described below may be used to fine tune the effector function of a present antibody, a ratio of the binding properties of the Fc region for the FcR (e.g., affinity and specificity), resulting in a therapeutic antibody with the desired properties for a particular disease indication and taking into consideration the biology of CXCR4.

It is understood that the Fc region as used herein includes the polypeptides comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cy2). Although the boundaries of the Fc region may vary, the human IgG heavy chain Fc region is usually defined to comprise residues C226 or P230 to its carboxyl-terminus, wherein the numbering is according to the EU index as set forth in Kabat. Fc may refer to this region in isolation, or this region in the context of an antibody, antigen binding fragment, or Fc fusion protein. Polymorphisms have been observed at a number of different Fc positions, including but not limited to positions 270, 272, 312, 315, 356, and 358 as numbered by the EU index, and thus slight differences between the presented sequence and sequences in the prior art may exist.

In one embodiment, the present disclosure encompasses Fc variant antibodies which have altered binding properties for an Fc ligand (e.g., an Fc receptor, C1q) relative to a native Fc antibody. Examples of binding properties include but are not limited to, binding specificity, equilibrium dissociation constant (K_{d}), dissociation and association rates (k_{off} and kₒₙ respectively), binding affinity and/or avidity. It is known in the art that the equilibrium dissociation constant (K_{d}) is defined as k_{off}/kₒₙ. In certain aspects, an antibody comprising an Fc variant region with a low K_{d} may be more desirable to an antibody with a high K_{d}. However, in some instances the value of the kₒₙ or k_{off} may be more relevant than the value of the K_{d}. One skilled in the art can determine which kinetic parameter is most important for a given antibody application. For example, a modification that reduces binding to one or more positive regulator (e.g., FcγRIIIA) and/or enhanced binding to an inhibitory Fc receptor (e.g., FcγRIIB) would be suitable for reducing ADCC activity. Accordingly, the ratio of binding affinities (e.g., the ratio of equilibrium dissociation constants (K_{d})) for different receptors can indicate if the ADCC activity of an Fc variant antibody of the disclosure is enhanced or decreased. Additionally, a modification that reduces binding to C1q would be suitable for reducing or eliminating CDC activity.

In one embodiment, Fc variant antibodies exhibit altered binding affinity for one or more Fc receptors including, but not limited to FcRn, FcγRI (CD64) including isoforms FcγRIA, FcγRIB, and FcγRIC; FcγRI (CD32 including isoforms FcγRIIA, FcγRIIB, and FcγRIIC); and FcγRIII (CD16, including isoforms FcγRIIIA and FcγRIIIB) as compared to an native Fc antibody.

In one embodiment, an Fc variant antibody has enhanced binding to one or more Fc ligand relative to a native Fc antibody. In another embodiment, the Fc variant antibody exhibits increased or decreased affinity for an Fc ligand that is at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or is between 2 fold and 10 fold, or between 5 fold and 50 fold, or between 25 fold and 100 fold, or between 75 fold and 200 fold, or between 100 and 200 fold, more or less than a native Fc antibody. In another embodiment, Fc variant antibodies exhibit affinities for an Fc ligand that are at least 90%, at least 80%, at least 70%, at least 60%, at least 50%, at least 40%, at least 30%, at least 20%, at least 10%, or at least 5% more or less than an native Fc antibody. In certain embodiments, an Fc variant antibody has increased affinity for an Fc ligand. In other embodiments, an Fc variant antibody has decreased affinity for an Fc ligand.

In a specific embodiment, an Fc variant antibody has enhanced binding to the Fc receptor FcγRIIIA. In another specific embodiment, an Fc variant antibody has enhanced binding to the Fc receptor FcγRIIB. In a further specific embodiment, an Fc variant antibody has enhanced binding to both the Fc receptors FcγRIIIA and FcγRIIB. In certain embodiments, Fc variant antibodies that have enhanced binding to FcγRIIIA do not have a concomitant increase in binding the FcγRIIB receptor as compared to a native Fc antibody. In a specific embodiment, an Fc variant antibody has reduced binding to the Fc receptor FcγRIIIA. In a further specific embodiment, an Fc variant antibody has reduced binding to the Fc receptor FcγRIIB. In still another specific embodiment, an Fc variant antibody exhibiting altered affinity for FcγRIIIA and/or FcγRIIB has enhanced binding to the Fc receptor FcRn. In yet another specific embodiment, an Fc variant antibody exhibiting altered affinity for FcγRIIIA and/or FcγRIIB has altered binding to C1q relative to a native Fc antibody.

In one embodiment, Fc variant antibodies exhibit affinities for FcγRIIIA receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or are between 2 fold and 10 fold, or between 5 fold and 50 fold, or between 25 fold and 100 fold, or between 75 fold and 200 fold, or between 100 and 200 fold, more or less than an native Fc antibody. In another embodiment, Fc variant antibodies exhibit affinities for FcγRIIIA that are at least 90%, at least 80%, at least 70%, at least 60%, at least 50%, at least 40%, at least 30%, at least 20%, at least 10%, or at least 5% more or less than an native Fc antibody.

In one embodiment, Fc variant antibodies exhibit affinities for FcγRIIB receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or are between 2 fold and 10 fold, or between 5 fold and 50 fold, or between 25 fold and 100 fold, or between 75 fold and 200 fold, or between 100 and 200 fold, more or less than an native Fc antibody. In another embodiment, Fc variant antibodies exhibit affinities for FcγRIIB that are at least 90%, at least 80%, at least 70%, at least 60%, at least 50%, at least 40%, at least 30%, at least 20%, at least 10%, or at least 5% more or less than an native Fc antibody.

In one embodiment, Fc variant antibodies exhibit increased or decreased affinities to C1q relative to a native Fc antibody. In another embodiment, Fc variant antibodies exhibit affinities for C1q receptor that are at least 2 fold, or at least 3 fold, or at least 5 fold, or at least 7 fold, or at least 10 fold, or at least 20 fold, or at least 30 fold, or at least 40 fold, or at least 50 fold, or at least 60 fold, or at least 70 fold, or at least 80 fold, or at least 90 fold, or at least 100 fold, or at least 200 fold, or are between 2 fold and 10 fold, or between 5 fold and 50 fold, or between 25 fold and 100 fold, or between 75 fold and 200 fold, or between 100 and 200 fold, more or less than an native Fc antibody. In another embodiment, Fc variant antibodies exhibit affinities for C1q that are at least 90%, at least 80%, at least 70%, at least 60%, at least 50%, at least 40%, at least 30%, at least 20%, at least 10%, or at least 5% more or less than an native Fc antibody. In still another specific embodiment, an Fc variant antibody exhibiting altered affinity for C1q has enhanced binding to the Fc receptor FcRn. In yet another specific embodiment, an Fc variant antibody exhibiting altered affinity for C1q has altered binding to FcγRIIIA and/or FcγRIIB relative to a native Fc antibody.

It is well known in the art that antibodies are capable of directing the attack and destruction of targeted antigen through multiple processes collectively known in the art as antibody effector functions. One of these processes, known as “antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to an antigen-bearing target cell and subsequently kill the target cell with cytotoxins. Specific high-affinity IgG antibodies directed to the surface of target cells “arm” the cytotoxic cells and are required for such killing. Lysis of the target cell is extracellular, requires direct cell-to-cell contact, and does not involve complement. Another process encompassed by the term effector function is complement dependent cytotoxicity (hereinafter referred to as “CDC”) which refers to a biochemical event of antibody-mediated target cell destruction by the complement system. The complement system is a complex system of proteins found in normal blood plasma that combines with antibodies to destroy pathogenic bacteria and other foreign cells. Still another process encompassed by the term effector function is antibody dependent cell-mediated phagocytosis (ADCP) which refers to a cell-mediated reaction wherein nonspecific cytotoxic cells that express one or more effector ligands recognize bound antibody on a target cell and subsequently cause phagocytosis of the target cell.

It is contemplated that Fc variant antibodies are characterized by in vitro functional assays for determining one or more FcγR mediated effector cell functions. In certain embodiments, Fc variant antibodies have similar binding properties and effector cell functions in in vivo models (such as those described and disclosed herein) as those in in vitro based assays. However, the present disclosure does not exclude Fc variant antibodies that do not exhibit the desired phenotype in in vitro based assays but do exhibit the desired phenotype in vivo.

The serum half-life of proteins comprising Fc regions may be increased by increasing the binding affinity of the Fc region for FcRn. The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body (or other mammal) or a specific compartment thereof, for example, as measured in serum, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The increase in half-life allows for the reduction in amount of drug given to a patient as well as reducing the frequency of administration. To increase the serum half life of the antibody, one may incorporate a salvage receptor binding epitope into the antibody (especially an antigen binding fragment) as described in U.S. Pat. No. 5,739,277, for example. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule. Alternatively, antibodies of the disclosure with increased half-lives may be generated by modifying amino acid residues identified as involved in the interaction between the Fc and the FcRn receptor (see, for examples, U.S. Pat. Nos. 6,821,505 and 7,083,784; and WO 09/058492). In addition, the half-life of antibodies of the disclosure may be increased by conjugation to PEG or Albumin by techniques widely utilized in the art. In some embodiments antibodies comprising Fc variant regions of the disclosure have an increased half-life of about 5%, about 10%, about 15%, about 20%, about 25%, about 30%, about 35%, about 40%, about 45%, about 50%, about 60%, about 65%, about 70%, about 80%, about 85%, about 90%, about 95%, about 100%, about 125%, about 150% or more as compared to an antibody comprising a native Fc region. In some embodiments antibodies comprising Fc variant regions have an increased half-life of about 2 fold, about 3 fold, about 4 fold, about 5 fold, about 10 fold, about 20 fold, about 50 fold or more, or is between 2 fold and 10 fold, or between 5 fold and 25 fold, or between 15 fold and 50 fold, as compared to an antibody comprising a native Fc region.

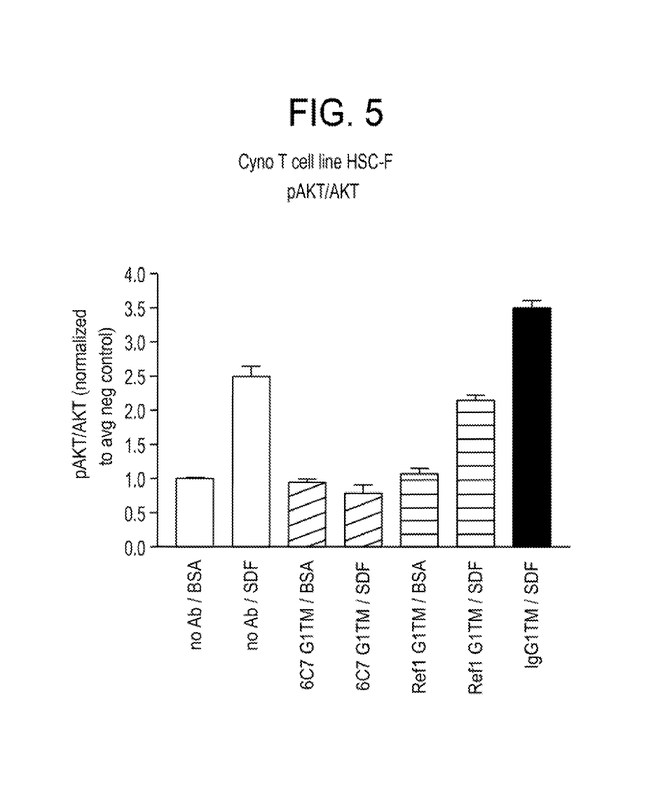

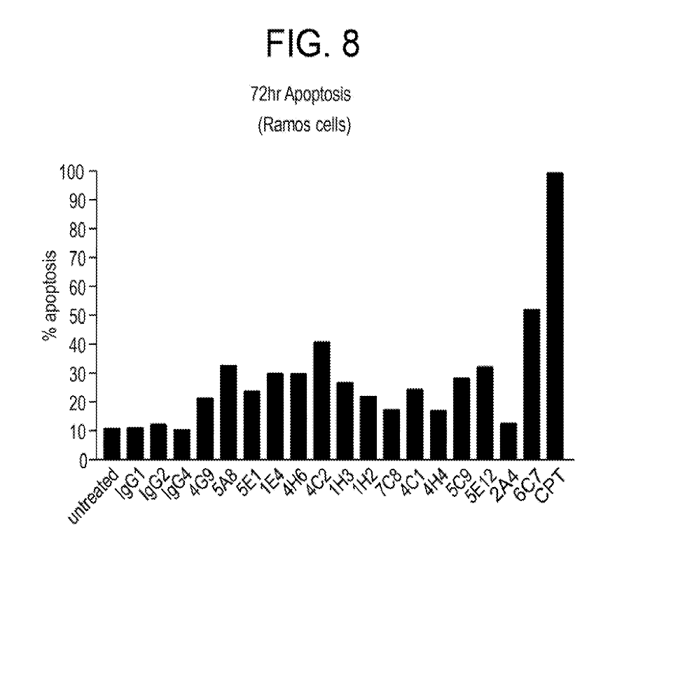

In one embodiment, the present disclosure provides Fc variants, wherein the Fc region comprises a modification (e.g., amino acid substitutions, amino acid insertions, amino acid deletions) at one or more positions selected from the group consisting of 221, 225, 228, 234, 235, 236, 237, 238, 239, 240, 241, 243, 244, 245, 247, 250, 251, 252, 254, 255, 256, 257, 262, 263, 264, 265, 266, 267, 268, 269, 279, 280, 284, 292, 296, 297, 298, 299, 305, 308, 313, 316, 318, 320, 322, 325, 326, 327, 328, 329, 330, 331, 332, 333, 334, 339, 341, 343, 370, 373, 378, 392, 416, 419, 421, 428, 433, 434, 435, 436, 440, and 443 as numbered by the EU index as set forth in Kabat. Optionally, the Fc region may comprise a modification at additional and/or alternative positions known to one skilled in the art (see, e.g., U.S. Pat. Nos. 5,624,821; 6,277,375; 6,737,056; 7,083,784; 7,317,091; 7,217,797; 7,276,585; 7,355,008; 2002/0147311; 2004/0002587; 2005/0215768; 2007/0135620; 2007/0224188; 2008/0089892; WO 94/29351; and WO 99/58572). Additional, useful amino acid positions and specific substitutions are exemplified in Tables 2, and 6-10 of U.S. Pat. No. 6,737,056; the tables presented in FIG. 41 of US 2006/024298; the tables presented in FIGS. 5, 12, and 15 of US 2006/235208; the tables presented in FIGS. 8, 9 and 10 of US 2006/0173170 and the tables presented in FIGS. 8-10, 13 and 14 of WO 09/058492.

In a specific embodiment, the present disclosure provides an Fc variant, wherein the Fc region comprises at least one substitution selected from the group consisting of 221K, 221Y, 225E, 225K, 225W, 228P, 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235E, 235F, 236E, 237L, 237M, 237P, 239D, 239E, 239N, 239Q, 239F, 239T, 239H, 239Y, 2401, 240A, 240T, 240M, 241W, 241 L, 241Y, 241E, 241R. 243W, 243L 243Y, 243R, 243Q, 244H, 245A, 247L, 247V, 247G, 250E, 250Q, 251F, 252L, 252Y, 254S, 254T, 255L, 256E, 256F, 256M, 257C, 257M, 257N, 2621, 262A, 262T, 262E, 2631, 263A, 263T, 263M, 264L, 2641, 264W, 264T, 264R, 264F, 264M, 264Y, 264E, 265A, 265G, 265N, 265Q, 265Y, 265F, 265V, 2651, 265L, 265H, 265T, 2661, 266A, 266T, 266M, 267Q, 267L, 268E, 269H, 269Y, 269F, 269R, 270E, 280A, 284M, 292P, 292L, 296E, 296Q, 296D, 296N, 296S, 296T, 296L, 2961, 296H, 296G, 297S, 297D, 297E, 298A, 298H, 2981, 298T, 298F, 2991, 299L, 299A, 299S, 299V, 299H, 299F, 299E, 3051, 308F313F, 316D, 318A, 318S, 320A, 320S, 322A, 322S, 325Q, 325L, 3251, 325D, 325E, 325A, 325T, 325V, 325H, 326A, 326D, 326E, 326G, 326M, 326V, 327G, 327W, 327N, 327L, 328S, 328M, 328D, 328E, 328N, 328Q, 328F, 3281, 328V, 328T, 328H, 328A, 329F, 329H, 329Q, 330K, 330G, 330T, 330C, 330L, 330Y, 330V, 3301, 330F, 330R, 330H, 331G, 331A, 331L, 331M, 331F, 331W, 331K, 331Q, 331E, 331S, 331V, 3311, 331C, 331Y, 331H, 331R, 331N, 331D, 331T, 332D, 332S, 332W, 332F, 332E, 332N, 332Q, 332T, 332H, 332Y, 332A, 333A, 333D, 333G, 333Q, 333S, 333V, 334A, 334E, 334H, 334L, 334M, 334Q, 334V, 334Y, 339T, 370E, 370N, 378D, 392T, 396L, 416G, 419H, 421K, 428L, 428F, 433K, 433L, 434A, 424F, 434W, 434Y, 436H, 440Y and 443W as numbered by the EU index as set forth in Kabat. Optionally, the Fc region may comprise additional and/or alternative amino acid substitutions known to one skilled in the art including but not limited to those exemplified in Tables 2, and 6-10 of U.S. Pat. No. 6,737,056; the tables presented in FIG. 41 of US 2006/024298; the tables presented in FIGS. 5, 12, and 15 of US 2006/235208; the tables presented in FIGS. 8, 9 and 10 of US 2006/0173170 and the tables presented in FIGS. 8, 9 and 10 of WO 09/058492.

In a specific embodiment, the present disclosure provides an Fc variant antibody, wherein the Fc region comprises at least one modification (e.g., amino acid substitutions, amino acid insertions, amino acid deletions) at one or more positions selected from the group consisting of 228, 234, 235 and 331 as numbered by the EU index as set forth in Kabat. In one embodiment, the modification is at least one substitution selected from the group consisting of 228P, 234F, 235E, 235F, 235Y, and 331S as numbered by the EU index as set forth in Kabat.

In another specific embodiment, the present disclosure provides an Fc variant antibody, wherein the Fc region is an IgG4 Fc region and comprises at least one modification at one or more positions selected from the group consisting of 228 and 235 as numbered by the EU index as set forth in Kabat. In still another specific embodiment, the Fc region is an IgG4 Fc region and the non-naturally occurring amino acids are selected from the group consisting of 228P, 235E and 235Y as numbered by the EU index as set forth in Kabat.

In another specific embodiment, the present disclosure provides an Fc variant, wherein the Fc region comprises at least one non-naturally occurring amino acid at one or more positions selected from the group consisting of 239, 330 and 332 as numbered by the EU index as set forth in Kabat. In one embodiment, the modification is at least one substitution selected from the group consisting of 239D, 330L, 330Y, and 332E as numbered by the EU index as set forth in Kabat.

In a specific embodiment, the present disclosure provides an Fc variant antibody, wherein the Fc region comprises at least one non-naturally occurring amino acid at one or more positions selected from the group consisting of 252, 254, and 256 as numbered by the EU index as set forth in Kabat. In one embodiment, the modification is at least one substitution selected from the group consisting of 252Y, 254T and 256E as numbered by the EU index as set forth in Kabat. See, U.S. Pat. No. 7,083,784, incorporated herein by reference in its entirety.

In certain embodiments the effector functions elicited by IgG antibodies strongly depend on the carbohydrate moiety linked to the Fc region of the protein (Claudia Ferrara et al., 2006, Biotechnology and Bioengineering 93:851-861). Thus, glycosylation of the Fc region can be modified to increase or decrease effector function (see for examples, Umana et al, 1999, Nat. Biotechnol 17:176-180; Davies et al., 2001, Biotechnol Bioeng 74:288-294; Shields et al, 2002, J Biol Chem 277:26733-26740; Shinkawa et al., 2003, J Biol Chem 278:3466-3473; U.S. Pat. Nos. 6,602,684; 6,946,292; 7,064,191; 7,214,775;7,393,683; 7,425,446; 7,504,256; U.S. Publication. Nos. 2003/0157108; 2003/0003097; 2009/0010921; POTILLEGENT™ technology (Biowa, Inc. Princeton, N.J.); GLYCOMAB™ glycosylation engineering technology (GLYCART biotechnology AG, Zurich, Switzerland)). Accordingly, in one embodiment the Fc regions of anti-CXCR4 antibodies of the disclosure comprise altered glycosylation of amino acid residues. In another embodiment, the altered glycosylation of the amino acid residues results in lowered effector function. In another embodiment, the altered glycosylation of the amino acid residues results in increased effector function. In a specific embodiment, the Fc region has reduced fucosylation. In another embodiment, the Fc region is afucosylated (see for examples, U.S. Patent Application Publication No. 2005/0226867). In one aspect, these antibodies with increased effector function, specifically ADCC, as generated in host cells (e.g., CHO cells, Lemna minor) engineered to produce highly defucosylated antibody with over 100-fold higher ADCC compared to antibody produced by the parental cells (Mori et al., 2004, Biotechnol Bioeng 88:901-908; Cox et al., 2006, Nat Biotechnol., 24:1591-7).

Addition of sialic acid to the oligosaccharides on IgG molecules can enhance their anti-inflammatory activity and alters their cytotoxicity (Keneko et al., Science, 2006, 313:670-673; Scallon et al., Mol. Immuno. 2007 March; 44(7):1524-34). The studies referenced above demonstrate that IgG molecules with increased sialylation have anti-inflammatory properties whereas IgG molecules with reduced sialylation have increased immunostimulatory properties (e.g., increase ADCC activity). Therefore, an antibody can be modified with an appropriate sialylation profile for a particular therapeutic application (US Publication No. 2009/0004179 and International Publication No. WO 2007/005786).

In one embodiment, the Fc regions of antibodies of the disclosure comprise an altered sialylation profile compared to the native Fc region. In one embodiment, the Fc regions of antibodies of the disclosure comprise an increased sialylation profile compared to the native Fc region. In another embodiment, the Fc regions of antibodies of the disclosure comprise a decreased sialylation profile compared to the native Fc region.

In one embodiment, the Fc variants of the present disclosure may be combined with other known Fc variants such as those disclosed in Ghetie et al., 1997, Nat Biotech. 15:637-40; Duncan et al, 1988, Nature 332:563-564; Lund et al., 1991, J. Immunol 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, J Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 7,122,637; 7,183,387; 7,332,581; 7,335,742; 7,371,826; 6,821,505; 6,180,377; 7,317,091; 7,355,008; 2004/0002587; and WO 99/58572. Other modifications and/or substitutions and/or additions and/or deletions of the Fc domain will be readily apparent to one skilled in the art.

In certain embodiments, an anti-CXCR4 antibody comprising a variant Fc region can comprise the variable heavy and/or variable light chains of the antibodies listed in Table 1 or disclosed in Table 7 or Table 8. In particular embodiments, the amino acid sequences of the heavy and light chains of an anti-CXCR4 antibody comprising a variant IgG1 Fc region corresponds to the following:

Anti-CXCR4 IgG1 TM Heavy Chain (6C7-TM):
(SEQ ID NO: 27)
QVQLVESGGGVVQPGRSLRLSCAASGFTFSNYVMHWVRQAPGKGLEWV
AVIWYDGSNKYYADSVKGRFTISRDNSKNTLSLQMNSLRAEDTAVYYC
ERGEGYYGSGSRYRGYYYGMDVWGQGTTVTVSSASTKGPSVFPLAPSS
KSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPP
CPAPEFEGGPSVFLFPPKPKDTLMISRTPEVTCVVVDVSHEDPEVKFN
WYVDGVEVHNAKTKPREEQYNSTYRVVSVLTVLHQDWLNGKEYKCKVS
NKALPASIEKTISKAKGQPREPQVYTLPPSREEMTKNQVSLTCLVKGF
YPSDIAVEWESNGQPENNYKTTPPVLDSDGSFFLYSKLTVDKSRWQQG
NVFSCSVMHEALHNHYTQKSLSLSPGK
Anti-CXCR4 Light Chain (6C7-TM):
(SEQ ID NO: 28)
DIQMTQSPSSLSASVGDRVTITCRASQGIRTDLGWYQQKPGKAPKRLI
YAASSLQSGVPSRFSGSGSGTEFTLTISSLQPEDFATYYCLQHNSYPR
TFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC

B. Glycosylation

In addition to the ability of glycosylation to alter the effector function of antibodies, modified glycosylation in the variable region can alter the affinity of the antibody for a target antigen. In one embodiment, the glycosylation pattern in the variable region of the present antibodies is modified. For example, an aglycoslated antibody can be made (i.e., the antibody lacks glycosylation). Glycosylation can be altered to, for example, increase the affinity of the antibody for a target antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861. One or more amino acid substitutions can also be made that result in elimination of a glycosylation site present in the Fc region (e.g., Asparagine 297 of IgG). Furthermore, aglycosylated antibodies may be produced in bacterial cells which lack the necessary glycosylation machinery.

C. Antibody Conjugates

In certain embodiments, the antibodies of the disclosure are conjugated or covalently attached to a substance using methods well known in the art. In one embodiment, the attached substance is a therapeutic agent, a detectable label (also referred to herein as a reporter molecule) or a solid support. Suitable substances for attachment to antibodies include, but are not limited to, an amino acid, a peptide, a protein, a polysaccharide, a nucleoside, a nucleotide, an oligonucleotide, a nucleic acid, a hapten, a drug, a hormone, a lipid, a lipid assembly, a synthetic polymer, a polymeric microparticle, a biological cell, a virus, a fluorophore, a chromophore, a dye, a toxin, a hapten, an enzyme, an antibody, an antigen binding fragment, a radioisotope, solid matrixes, semi-solid matrixes and combinations thereof. Methods for conjugation or covalently attaching another substance to an antibody are well known in the art.

In certain embodiments, the antibodies of the disclosure are conjugated to a solid support. Antibodies may be conjugated to a solid support as part of the screening and/or purification and/or manufacturing process. Alternatively antibodies of the disclosure may be conjugated to a solid support as part of a diagnostic method or composition. A solid support suitable for use in the present disclosure is typically substantially insoluble in liquid phases. A large number of supports are available and are known to one of ordinary skill in the art. Thus, solid supports include solid and semi-solid matrixes, such as aerogels and hydrogels, resins, beads, biochips (including thin film coated biochips), microfluidic chip, a silicon chip, multi-well plates (also referred to as microtitre plates or microplates), membranes, conducting and nonconducting metals, glass (including microscope slides) and magnetic supports. More specific examples of solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

In some embodiments, the solid support may include a reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the antibodies of the disclosure.

A suitable solid phase support can be selected on the basis of desired end use and suitability for various synthetic protocols. For example, where amide bond formation is desirable to attach the antibodies of the disclosure to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TENTAGEL™, Rapp Polymere, Tubingen, Germany), polydimethyl-acrylamide resin (available from Milligen/Biosearch, California), or PEGA beads (obtained from Polymer Laboratories).

In certain embodiments, the antibodies of the disclosure are conjugated to labels for purposes of diagnostics and other assays wherein the antibody and/or its associated ligand may be detected. A label conjugated to an antibody and used in the present methods and compositions described herein, is any chemical moiety. Labels include, without limitation, a chromophore, a fluorophore, a fluorescent protein, a phosphorescent dye, a tandem dye, a particle, a hapten, an enzyme and a radioisotope.

In certain embodiments, the anti-CXCR4 antibodies are conjugated to a fluorophore. As such, fluorophores used to label antibodies of the disclosure include, without limitation; a pyrene (including any of the corresponding derivative compounds disclosed in U.S. Pat. No. 5,132,432), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine (including any corresponding compounds in US Pat. Nos. 6,977,305 and 6,974,873), a carbocyanine (including any corresponding compounds in U.S. Ser. No. 09/557,275; U.S.; Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; and publications WO 02/26891, WO 97/40104, WO 99/51702, WO 01/21624; EP 1 065 250 A1), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343; 5,227,487; 5,442,045; 5,798,276; 5,846,737; 4,945,171; U.S. Ser. Nos. 09/129,015 and 09/922,333), an oxazine (including any corresponding compounds disclosed in U.S. Pat. No. 4,714,763) or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in 5,242,805), aminooxazinones, diaminooxazines, and their benzo-substituted analogs.

In a specific embodiment, the fluorophores conjugated to the antibodies described herein include xanthene (rhodol, rhodamine, fluorescein and derivatives thereof) coumarin, cyanine, pyrene, oxazine and borapolyazaindacene. In other embodiments, such fluorophores are sulfonated xanthenes, fluorinated xanthenes, sulfonated coumarins, fluorinated coumarins and sulfonated cyanines. Also included are dyes sold under the tradenames, and generally known as, Alexa Fluor, DyLight, Cy Dyes, BODIPY, Oregon Green, Pacific Blue, IRDyes, FAM, FITC, and ROX.

The choice of the fluorophore attached to the anti-CXCR4 antibody will determine the absorption and fluorescence emission properties of the conjugated antibody. Physical properties of a fluorophore label that can be used for antibody and antibody bound ligands include, but are not limited to, spectral characteristics (absorption, emission and stokes shift), fluorescence intensity, lifetime, polarization and photo-bleaching rate, or combination thereof. All of these physical properties can be used to distinguish one fluorophore from another, and thereby allow for multiplexed analysis. In certain embodiments, the fluorophore has an absorption maximum at wavelengths greater than 480 nm. In other embodiments, the fluorophore absorbs at or near 488 nm to 514 nm (particularly suitable for excitation by the output of the argon-ion laser excitation source) or near 546 nm (particularly suitable for excitation by a mercury arc lamp). In other embodiment a fluorophore can emit in the NIR (near infra red region) for tissue or whole organism applications. Other desirable properties of the fluorescent label may include cell permeability and low toxicity, for example if labeling of the antibody is to be performed in a cell or an organism (e.g., a living animal).

In certain embodiments, an enzyme is a label and is conjugated to an anti-CXCR4 antibody. Enzymes are desirable labels because amplification of the detectable signal can be obtained resulting in increased assay sensitivity. The enzyme itself does not produce a detectable response but functions to break down a substrate when it is contacted by an appropriate substrate such that the converted substrate produces a fluorescent, colorimetric or luminescent signal. Enzymes amplify the detectable signal because one enzyme on a labeling reagent can result in multiple substrates being converted to a detectable signal. The enzyme substrate is selected to yield the preferred measurable product, e.g. colorimetric, fluorescent or chemiluminescence. Such substrates are extensively used in the art and are well known by one skilled in the art.

In one embodiment, colorimetric or fluorogenic substrate and enzyme combination uses oxidoreductases such as horseradish peroxidase and a substrate such as 3,3′-diaminobenzidine (DAB) and 3-amino-9-ethylcarbazole (AEC), which yield a distinguishing color (brown and red, respectively). Other colorimetric oxidoreductase substrates that yield detectable products include, but are not limited to: 2,2-azino-bis(3-ethylbenzothiazoline-6-sulfonic acid) (ABTS), o-phenylenediamine (OPD), 3,3′,5,5′-tetramethylbenzidine (TMB), o-dianisidine, 5-aminosalicylic acid, 4-chloro-1-naphthol. Fluorogenic substrates include, but are not limited to, homovanillic acid or 4-hydroxy-3-methoxyphenylacetic acid, reduced phenoxazines and reduced benzothiazines, including AMPLEX Red reagent and its variants (U.S. Pat. No. 4,384,042) and reduced dihydroxanthenes, including dihydrofluoresceins (U.S. Pat. No. 6,162,931) and dihydrorhodamines including dihydrorhodamine 123. Peroxidase substrates that are tyramides (U.S. Pat. Nos. 5,196,306; 5,583,001 and 5,731,158) represent a unique class of peroxidase substrates in that they can be intrinsically detectable before action of the enzyme but are “fixed in place” by the action of a peroxidase in the process described as tyramide signal amplification (TSA). These substrates are extensively utilized to label targets in samples that are cells, tissues or arrays for their subsequent detection by microscopy, flow cytometry, optical scanning and fluorometry.

In another embodiment, a colorimetric (and in some cases fluorogenic) substrate and enzyme combination uses a phosphatase enzyme such as an acid phosphatase, an alkaline phosphatase or a recombinant version of such a phosphatase in combination with a colorimetric substrate such as 5-bromo-6-chloro-3-indolyl phosphate (BCIP), 6-chloro-3-indolyl phosphate, 5-bromo-6-chloro-3-indolyl phosphate, p-nitrophenyl phosphate, or o-nitrophenyl phosphate or with a fluorogenic substrate such as 4-methylumbelliferyl phosphate, 6,8-difluoro-7-hydroxy-4-methylcoumarinyl phosphate (DiFMUP, U.S. Pat. No. 5,830,912) fluorescein diphosphate, 3-O-methylfluorescein phosphate, resorufin phosphate, 9H-(1,3-dichloro-9,9-dimethylacridin-2-one-7-yl)phosphate (DDAO phosphate), or ELF 97, ELF 39 or related phosphates (U.S. Pat. Nos. 5,316,906 and 5,443,986).

Glycosidases, in particular beta-galactosidase, beta-glucuronidase and beta-glucosidase, are additional suitable enzymes. Appropriate colorimetric substrates include, but are not limited to, 5-bromo-4-chloro-3-indolylbeta-D-galactopyranoside (X-gal) and similar indolyl galactosides, glucosides, and glucuronides, o-nitrophenyl beta-D-galactopyranoside (ONPG) and p-nitrophenyl beta-D-galactopyranoside. In one embodiment, fluorogenic substrates include resorufin beta-D-galactopyranoside, fluorescein digalactoside (FDG), fluorescein diglucuronide and their structural variants (U.S. Pat. Nos. 5,208,148; 5,242,805; 5,362,628; 5,576,424 and 5,773,236), 4-methylumbelliferyl beta-D-galactopyranoside, carboxyumbelliferyl beta-D-galactopyranoside and fluorinated coumarin beta-D-galactopyranosides (U.S. Pat. No. 5,830,912).

Additional enzymes include, but are not limited to, hydrolases such as cholinesterases and peptidases, oxidases such as glucose oxidase and cytochrome oxidases, and reductases for which suitable substrates are known.

Enzymes and their appropriate substrates that produce chemiluminescence are preferred for some assays. These include, but are not limited to, natural and recombinant forms of luciferases and aequorins. Chemiluminescence-producing substrates for phosphatases, glycosidases and oxidases such as those containing stable dioxetanes, luminol, isoluminol and acridinium esters are additionally useful.

In another embodiment, haptens such as biotin, are also utilized as labels. Biotin is useful because it can function in an enzyme system to further amplify the detectable signal, and it can function as a tag to be used in affinity chromatography for isolation purposes. For detection purposes, an enzyme conjugate that has affinity for biotin is used, such as avidin-HRP. Subsequently a peroxidase substrate is added to produce a detectable signal.

Haptens also include hormones, naturally occurring and synthetic drugs, pollutants, allergens, affector molecules, growth factors, chemokines, cytokines, lymphokines, amino acids, peptides, chemical intermediates, nucleotides and the like.

In certain embodiments, fluorescent proteins may be conjugated to the antibodies as a label. Examples of fluorescent proteins include green fluorescent protein (GFP) and the phycobiliproteins and the derivatives thereof. The fluorescent proteins, especially phycobiliprotein, are particularly useful for creating tandem dye labeled labeling reagents. These tandem dyes comprise a fluorescent protein and a fluorophore for the purposes of obtaining a larger stokes shift wherein the emission spectra is farther shifted from the wavelength of the fluorescent protein's absorption spectra. This is particularly advantageous for detecting a low quantity of a target in a sample wherein the emitted fluorescent light is maximally optimized, in other words little to none of the emitted light is reabsorbed by the fluorescent protein. For this to work, the fluorescent protein and fluorophore function as an energy transfer pair wherein the fluorescent protein emits at the wavelength that the fluorophore absorbs at and the fluorphore then emits at a wavelength farther from the fluorescent proteins than could have been obtained with only the fluorescent protein. A particularly useful combination is the phycobiliproteins disclosed in U.S. Pat. Nos. 4,520,110; 4,859,582; 5,055,556 and the sulforhodamine fluorophores disclosed in U.S. Pat. No. 5,798,276, or the sulfonated cyanine fluorophores disclosed in U.S. Pat. Nos. 6,977,305 and 6,974,873; or the sulfonated xanthene derivatives disclosed in U.S. Pat. No. 6,130,101 and those combinations disclosed in U.S. Pat. No. 4,542,104. Alternatively, the fluorophore functions as the energy donor and the fluorescent protein is the energy acceptor.

In certain embodiments, the label is a radioactive isotope. Examples of suitable radioactive materials include, but are not limited to, iodine (¹²¹I, ¹²³I, ¹²⁵I, ¹³¹I), carbon (¹⁴C), sulfur (³⁵S), tritium (³H), indium (¹¹¹In, ¹¹²In, ¹¹³mIn, ¹¹⁵mIn), technetium (⁹⁹Tc, ⁹⁹mTc), thallium (²⁰¹Ti), gallium (⁶⁸Ga, ⁶⁷Ga), palladium (¹⁰³Pd), molybdenum (⁹⁹Mo), xenon (¹³⁵Xe), fluorine (18F, ¹⁵³SM, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh and ⁹⁷Ru.

### (v) Methods of Use

(a) Diagnostic Methods of Use

In certain embodiments, the anti-CXCR4 antibodies (including fragments) and compositions thereof of the disclosure may be used in vivo and/or in vitro for detecting CXCR4 expression in cells and tissue or for imaging CXCR4 expressing cells and tissues. In certain embodiments, the antibodies are human antibodies and such antibodies are used to image CXCR4 expression in a living human patient. Given that the anti-CXCR4 antibodies or antigen binding fragments described herein specifically bind to human CXCR4, these antibodies can be used to detect or image CXCR4 expression in living patients.

By way of example, diagnostic uses can be achieved, for example, by contacting a sample to be tested, optionally along with a control sample, with the antibody under conditions that allow for formation of a complex between the antibody and CXCR4. Complex formation is then detected (e.g., using an ELISA or by imaging to detect a moiety attached to the antibody). When using a control sample along with the test sample, complex is detected in both samples and any statistically significant difference in the formation of complexes between the samples is indicative of the presence of CXCR4 in the test sample.

In one embodiment, the disclosure provides a method of determining the presence of CXCR4 in a sample suspected of containing CXCR4, said method comprising exposing the sample to an anti-CXCR4 antibody of the disclosure, and determining binding of the antibody to CXCR4 in the sample wherein binding of the antibody to CXCR4 in the sample is indicative of the presence of the CXCR4 in the sample. In one embodiment, the sample is a biological sample.

In certain embodiments, the anti-CXCR4 antibodies may be used to detect the overexpression or amplification of CXCR4 using an in vivo diagnostic assay. In one embodiment, the anti-CXCR4 antibody is added to a sample wherein the antibody binds the CXCR4 to be detected and is tagged with a detectable label (e.g. a radioactive isotope or a fluorescent label) and externally scanning the patient for localization of the label.

Alternatively, or additionally, FISH assays such as the INFORM™ (sold by Ventana, Ariz.) or PATHVISION™ (Vysis, Ill.) may be carried out on formalin-fixed, paraffin-embedded tissue to determine the extent (if any) of CXCR4 expression or overexpression in a sample.

(b) Therapeutic Methods of Uses

In certain aspects, the anti-CXCR4 antibodies (including antigen binding fragments) and compositions thereof of the disclosure may be administered for prevention and/or treatment of cancer in a subject in need thereof. The disclosure encompasses methods of preventing, treating, maintaining, ameliorating, or inhibiting cancer and/or preventing and/or alleviating one or more symptoms of the disease in a mammal, comprising administering a therapeutically effective amount of the anti-CXCR4 antibody to the mammal. Symptoms can include, for example, pain associated with cancer, or manifestations of physiological functions disrupted by the presence of cancer. Symptoms can be measured, for example, by laboratory assays routinely used to measure physiological functions, or standard patient questionnaires used to measure symptoms such as pain.

In certain aspects, the disclosure provides a method of treating and/or preventing human cancer or cancer cell growth or tumor growth or tumor metastasis in a subject in need thereof, comprising administering to said subject a therapeutically effective amount of the antibody or antigen binding fragment of the disclosure. In certain embodiments, the cancer is ovarian cancer. In other embodiments, the cancer is breast cancer. In still other embodiments, the cancer is prostate cancer or lung cancer. In still other embodiments, the cancer is non-Hodgkins lymphoma (NHL), multiple myeloma (MM), diffuse large B-cell lymphoma (DLBL), follicular lymphoma, large B-cell lymphoma or chronic lymphocytic leukemia (CLL) or chronic myelogenous leukemia (CML). In other embodiments, the method comprises treating bone metastatic cancer, particularly bone metastatic prostate or breast cancer. In certain embodiments, the method is part of a therapeutic regimen for treating ovarian cancer, or any of the foregoing cancers. In certain embodiments, the antibody or antigen binding fragment is administered to inhibit angiogenesis, such as angiogenesis associate with a cancer or tumor. The disclosure contemplates that these cancers may be treated using an anti-CXCR4 antibody as a monotherapy, or using anti-CXCR4 as part of a combination therapy in which one or more other agents or treatment modalities are administered. In the case of combination therapy, the other agent or modality may be administered at the same or at differing times. In certain embodiments, an anti-CXCR4 antibody of the disclosure is used in combination (whether administered before, after or at the same time) with the standard of care for the particular cancer.

In certain aspects, the disclosure provides a method for inhibiting cell growth and/or metastasis of a cancer cell expressing human CXCR4, comprising contacting the cell with the antibody or antigen binding fragment of the disclosure or otherwise administering the antibody or antigen binding fragment to a patient in need thereof

In other aspects, the disclosure provides a method for increasing stem cell mobilization. Such a method is used prior to or following transplantation.

Any of the anti-CXCR4 antibodies or antigen binding fragments having any one or more of the structural and functional features described herein can be used in a method of treating a human or animal patient in need thereof. Throughout this portion of the specification, it should be understood that the disclosure contemplates that any of the CXCR4 antibodies or antigen binding fragments disclosed herein, including antibodies having any one or more of the structural and/or functional features described herein, can be used in a method of treating a patient in need thereof

The antibodies may be used alone or used as part of a therapeutic regimen specific to the particular underlying cancer being treated. For example, additional treatment modalities that can be used include, but are not limited to, other agents, radiation, surgery, acupuncture, massage, hormone therapy, narcotics, analgesics, and the like. Additionally or alternatively, the antibodies may be used alone or used as part of a regimen for managing symptoms of cancer, such as pain.

The anti-tumour treatment defined herein may be applied as a sole therapy or may involve, in addition to the compounds of the disclosure, other agents, conventional surgery, bone marrow and peripheral stem cell transplantations or radiotherapy or chemotherapy.

In certain embodiments, a suitable therapeutic regimen includes one or more agents, in addition to an anti-CXCR4 antibody of the disclosure, possessing a pharmaceutical property selected from anti-mitotic, alkylating, anti-metabolite, anti-angiogenic, apoptotic, alkaloid, COX-2, and antibiotic agents and combinations thereof. By way of example, in certain embodiments, the drug can be selected from the group of nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, anti-metabolites, antibiotics, enzymes, epipodophyllotoxins, platinum coordination complexes, vinca alkaloids, substituted ureas, methyl hydrazine derivatives, adrenocortical suppressants, endostatin, taxols, camptothecins, oxaliplatin, doxorubicins and their analogs, and a combination thereof.

Further non-limiting examples of agents of use as part of a therapeutic regimen for treating cancerous conditions, such as any of the cancerous conditions described herein, include anthracyclines, such as doxorubicin (adriamycin), daunorubicin (daunomycin), idarubicin, detorubicin, caminomycin, epirubicin, esorubicin, and morpholino and substituted derivatives, combinations and modifications thereof. Further examples of agents of use as part of a therapeutic regimen for treating cancerous conditions, such as any of the cancerous conditions described herein, include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof. In certain embodiments, the agent is doxorubicin, morpholinodoxorubicin, or morpholinodaunorubicin. As noted herein, therapeutic regimens may include any one or more additional agents and/or any one or more additional therapeutic modalities. Although, in certain embodiments, the anti-CXCR4 antibody of the disclosure is administered as a monotherapy, and the regimen does not include further therapies.

To illustrate briefly, below is provided a list of other agents that can be used, alone or in combination with each other and/or with other therapies, as part of a combination method.

Suitable agents include:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); anti-metabolites (for example gemcitabine and antifolates such as fluoropyrimidines like 5-fluorouracil and tegafur, raltitrexed, methotrexate, cytosine arabinoside, and hydroxyurea); antitumor antibiotics (for example anthracyclines like adriamycin, bleomycin, doxorubicin, daunomycin, epirubicin, idarubicin, mitomycin-C, dactinomycin and mithramycin); anti-mitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5-α-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethox-y]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-met-hylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or, inhibitors of cathepsins, inhibitors of serine proteases for example matriptase, hepsin, urokinase, inhibitors of heparanase;

(iv) cytotoxic agents such as fludarabine, 2-chlorodeoxyadenosine, chlorambucil or doxorubicin and combination thereof such as Fludarabine+cyclophosphamide, CVP: cyclophosphamide+vincristine+prednisone, ACVBP: doxorubicin+cyclophosphamide+vindesine+bleomycin+prednisone, CHOP: cyclophosphamide+doxorubicin+vincristine+prednisone, CNOP: cyclophosphamide+mitoxantrone+vincristine+prednisone, m-BACOD: methotrexate+bleomycin+doxorubicin+cyclophosphamide+vincristine+dexamethasone+leucovorin, MACOP-B: methotrexate+doxorubicin+cyclophosphamide+vincristine+prednisone fixed dose+bleomycin+leucovorin, or ProMACE CytaBOM: prednisone+doxorubicin+cyclophosphamide+etoposide+cytarabine+bleomycin+vincristine+methotrexate+leucovorin;

(v) inhibitors of growth factor function, for example such inhibitors include growth factor antibodies and growth factor receptor antibodies (for example the anti-erbB2 antibody trastuzumab [Herceptin®], the anti-EGFR antibody panitumumab, the anti-erbB1 antibody cetuximab [Erbitux] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazol-in-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors, aurora kinase inhibitors (for example AZD1152, PH739358, VX-680, MLN8054, R763, MP235, MP529, VX-528 and AX39459), cyclin dependent kinase inhibitors such as CDK2 and/or CDK4 inhibitors, and inhibitors of survival signaling proteins such as Bcl-2, Bcl-XL for example ABT-737;

(vi) antiangiogenic agents such as those which inhibit the effects of vascular endothelial growth factor, [for example the anti-vascular endothelial cell growth factor antibody bevacizumab (Avastin®) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)-quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856, WO 98/13354, WO00/47212 and WO01/32651 and compounds that work by other mechanisms (for example linomide, inhibitors of integrin .alpha.v.beta.3 function and angiostatin)] or colony stimulating factor 1 (CSF1) or CSF1 receptor;

(vii) vascular damaging agents such as Combretastatin A4 and compounds disclosed in International Patent Applications WO 99/02166, WO 00/40529, WO 00/41669, WO 01/92224, WO 02/04434 and WO 02/08213;

(viii) antisense therapies, for example those which are directed to the targets listed above, such as G-3139 (Genasense), an anti bc12 antisense;

(ix) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(x) immunotherapy approaches, including for example treatment with Alemtuzumab (campath-1H®), a monoclonal antibody directed at CD52, or treatment with antibodies directed at CD22, ex vivo and in vivo approaches to increase the immunogenicity of patient tumour cells, transfection with cytokines such as interleukin 2, interleukin 4 or granulocyte macrophage colony stimulating factor, approaches to decrease T cell anergy such as treatment with monoclonal antibodies inhibiting CTLA-4 function, approaches using transfected immune cells such as cytokine transfected dendritic cells, approaches using cytokine transfected tumour cell lines and approaches using anti idiotypic antibodies;

(xi) inhibitors of protein degradation such as proteasome inhibitor such as Velcade (bortezomid).

For any methods of treating involving administering a combination of agents and/or therapies, such conjoint treatment may be achieved by way of the simultaneous, sequential or separate dosing of the individual components of the treatment. Such combination products employ the compounds of this disclosure, or pharmaceutically acceptable salts thereof, within the dosage range described hereinbefore and the other pharmaceutically active agent within its approved dosage range.

Still further embodiments of the disclosure include methods of treating a proliferative, angiogenic, cell adhesion or invasion-related disease in an animal by administering to the animal a therapeutically effective dose of antibody of the disclosure. In certain embodiments, the method further comprises selecting an animal in need of treatment for a proliferative, angiogenic, cell adhesion or invasion-related disease, and administering to the animal a therapeutically effective dose of an antibody of the disclosure. In certain embodiments, the animal is human. In certain embodiments, the antibody is a fully human monoclonal antibody. In certain embodiments, the antibody is an antibody of the disclosure selected from the group consisting of 2A4, 4C1, 5C9, 5E1, 6C7 or 7C8. In other embodiments, the antibody is an antibody of the disclosure having any one or more (1, 2, 3, 4, 5, 6, 7, 8, 9, etc.) of the functional and/or structural characteristics of the CXCR4 antibodies disclosed herein.

Still further embodiments of the disclosure include methods of inhibiting CXCR4-induced cell proliferation, angiogenesis, cell adhesion and/or invasion-related disease in an animal by administering to the animal a therapeutically effective dose of an antibody of the disclosure. In certain embodiments the method further comprises selecting an animal in need of treatment for CXCR4 induced cell proliferation, angiogenesis, cell adhesion and/or invasion-related disease, and administering to said animal a therapeutically effective dose of an antibody of the disclosure. In certain embodiments, the animal is human. In certain embodiments, the antibody of the disclosure is a fully human monoclonal antibody. In certain embodiments, the antibody of the disclosure may be selected from the group consisting of 2A4, 4C1, 5C9, 5E1, 6C7 or 7C8. In other embodiments, the antibody is an antibody of the disclosure having any one or more (1, 2, 3, 4, 5, 6, 7, 8, 9, etc.) of the functional and/or structural characteristics of the CXCR4 antibodies disclosed herein.

Still further embodiments of the disclosure include methods of inhibiting tumour cell adhesion, motility, invasion, cellular metastasis, tumour growth or angiogenesis in an animal by administering to the animal a therapeutically effective dose of an antibody of the disclosure. In certain embodiments, the method further comprises selecting an animal in need of treatment for tumour cell adhesion, motility, invasion, cellular metastasis, tumour growth or angiogenesis, and administering to the animal a therapeutically effective dose of an antibody of the disclosure. In certain embodiments, the animal is human. In certain embodiments, the antibody of the disclosure is a fully human monoclonal antibody. In certain embodiments, the antibody of the disclosure is selected from the group consisting of 2A4, 4C1, 5C9, 5E1, 6C7 or 7C8. In other embodiments, the antibody is an antibody of the disclosure having any one or more (1, 2, 3, 4, 5, 6, 7, 8, 9, etc.) of the functional and/or structural characteristics of the CXCR4 antibodies disclosed herein.

Still further embodiments of the disclosure include methods of treating an animal suffering from a neoplastic disease by administering to the animal a therapeutically effective dose of an antibody of the disclosure. In certain embodiments the method further comprises selecting an animal in need of treatment for a neoplastic disease, and administering to the animal a therapeutically effective dose of an antibody of the disclosure.

Still further embodiments of the disclosure include methods of treating an animal suffering from a non-neoplastic disease by administering to the animal a therapeutically effective dose of an antibody of the disclosure. In certain embodiments the method further comprises selecting an animal in need of treatment for a non-neoplastic disease, and administering to the animal a therapeutically effective dose of an antibody of the disclosure.

Still further embodiments of the disclosure include methods of treating an animal suffering from a malignant tumour by administering to the animal a therapeutically effective dose of an antibody of the disclosure. In certain embodiments, the method further comprises selecting an animal in need of treatment for a malignant tumour, and administering to the animal a therapeutically effective dose of an antibody of the disclosure.

Still further embodiments of the disclosure include methods of treating an animal suffering from a disease or condition associated with CXCR4 expression by administering to the animal a therapeutically effective dose of an antibody of the disclosure. In certain embodiments the method further comprises selecting an animal in need of treatment for a disease or condition associated with CXCR4 expression, and administering to the animal a therapeutically effective dose of an antibody the disclosure. CXCR4 expression can be determined, for example, by FACS analysis on isolated cells such as peripheral blood mononuclear cells (PBMCs) or by by immunostaining on isolated cells using an anti-CXCR4 antibody

Treatable proliferative, angiogenic, cell adhesion or invasion-related diseases include neoplastic diseases. Disease-related cell adhesion and/or invasion and/or angiogenesis and/or proliferation may be any abnormal, undesirable or pathological cell adhesion and/or invasion and/or angiogenesis and/or proliferation, for example tumour-related cell adhesion and/or invasion and/or angiogenesis and/or proliferation.

In one embodiment the present disclosure is suitable for use in inhibiting CXCR4, in patients with a tumour which is dependent alone, or in part, on CXCR4. In certain embodiments, the tumor is associated with breast cancer or ovarian cancer.

In certain embodiments, the method is a method of treating a cancer or malignant tumour selected from breast, ovarian, lung, prostate, CL, NHL, or MM. In certain embodiments, the method is a method of treating bone metastatic prostate or breast cancer.

In certain embodiments, the disclosure provides a method of treating breast, ovarian, lung, or prostate cancer comprising administering an anti-CXCR4 antibody of the disclosure as a single agent therapy. In other embodiments, the disclosure provides a method of treating breast, ovarian, lung, or prostate cancer comprising administering an anti-CXCR4 antibody of the disclosure as part of a combination therapy together with one or more agents that constitute the standard of care for the particular cancer and stage of disease. In certain embodiments, the combination therapy for breast cancer includes a taxane, such as paclitaxel or docetaxel. In certain embodiments, the combination for prostate cancer includes a taxane, such as paclitaxel or docetaxel. In certain embodiments, the combination therapy for lung cancer includes a platinum drug, such as cisplatin, carboplatin or oxaliplatin.

In certain embodiments, the disclosure provides a method of treating CLL, NHL or MM comprising administering an anti-CXCR4 antibody of the disclosure as a single agent therapy. In other embodiments, the disclosure provides a method of treating CLL, NHL or MM comprising administering an anti-CXCR4 antibody of the disclosure as part of a combination therapy together with one or more agents that constitute the standard of care for the particular cancer and stage of disease. An example of a standard of care agent is Rituxan®.

In certain embodiments, the disclosure provides a method of treating bone metastatic prostate or breast cancer comprising administering an anti-CXCR4 antibody of the disclosure as a single agent therapy. In other embodiments, the disclosure provides a method of treating bone metastatic prostate or breast cancer comprising administering an anti-CXCR4 antibody of the disclosure as part of a combination therapy together with one or more agents that constitute the standard of care for the particular cancer and stage of disease.

In certain embodiments the present disclosure is suitable for use in inhibiting CXCR4, in patients with inflammation which is dependent alone, or in part, on CXCR4.

Still further embodiments of the disclosure include use of an antibody of the disclosure in the preparation of a medicament for the treatment of an animal suffering from a proliferative, angiogenic, cell adhesion or invasion-related disease. In certain embodiments, the use further comprises selecting an animal in need of treatment for a proliferative, angiogenic, cell adhesion or invasion-related disease.

Still further embodiments of the disclosure include use of an antibody of the disclosure in the preparation of medicament for the treatment of CXCR4-induced cell proliferation, angiogenesis, cell adhesion and/or invasion-related disease in an animal. In certain embodiments the use further comprises selecting an animal in need of treatment for a CXCR4-induced proliferative, angiogenic, cell adhesion and/or invasion-related disease.

Still further embodiments of the disclosure include use of an antibody of the disclosure in the preparation of medicament for the treatment of tumour cell adhesion, motility, invasion, cellular metastasis, tumour growth or angiogenesis in an animal. In certain embodiments the use further comprises selecting an animal in need of treatment for tumour cell adhesion, motility, invasion, cellular metastasis, tumour growth or angiogenesis.

Still further embodiments of the disclosure include use of an antibody of the disclosure in the preparation of a medicament for the treatment of an animal suffering from a neoplastic disease. In certain embodiments the use further comprises selecting an animal in need of treatment for a neoplastic disease.

Still further embodiments of the disclosure include use of a targeted binding agent or antibody of the disclosure in the preparation of a medicament for the treatment of an animal suffering from a non-neoplastic disease. In certain embodiments the use further comprises selecting an animal in need of treatment for a non-neoplastic disease.

Still further embodiments of the disclosure include use of a targeted binding agent or antibody of the disclosure in the preparation of a medicament for the treatment of an animal suffering from a malignant tumour. In certain embodiments the use further comprises selecting an animal in need of treatment for a malignant tumour.

Still further embodiments of the disclosure include use of a targeted binding agent or antibody of the disclosure in the preparation of a medicament for the treatment of an animal suffering from a disease or condition associated with CXCR4 expression. In certain embodiments the use further comprises selecting an animal in need of treatment for a disease or condition associated with CXCR4 expression.

Still further embodiments of the disclosure include a targeted binding agent or antibody of the disclosure for use as a medicament for the treatment of an animal suffering from a proliferative, angiogenic, cell adhesion or invasion-related disease.

Still further embodiments of the disclosure include a targeted binding agent or antibody of the disclosure for use as a medicament for the treatment of an animal suffering from tumour cell adhesion, motility, invasion, cellular metastasis, tumour growth or angiogenesis in an animal.

Still further embodiments of the disclosure include a targeted binding agent or antibody of the disclosure for use as a medicament for the treatment of an animal suffering from a neoplastic disease.

Still further embodiments of the disclosure include a targeted binding agent or antibody of the disclosure for use as a medicament for the treatment of an animal suffering from a malignant tumour.

Still further embodiments of the disclosure include a targeted binding agent or antibody of the disclosure for use as a medicament for the treatment of an animal suffering from a disease or condition associated with CXCR4 expression.

In one embodiment treatment of a proliferative, angiogenic, cell adhesion or invasion-related disease; a neoplastic disease; a malignant tumour; or a disease or condition associated with CXCR4 expression, comprises managing, ameliorating, preventing, any of the aforementioned diseases or conditions.

In one embodiment treatment of a neoplastic disease comprises inhibition of tumour growth, tumour growth delay, regression of tumour, shrinkage of tumour, increased time to regrowth of tumour on cessation of treatment, increased time to tumour recurrence, slowing of disease progression.

In one embodiment treatment of a disease or condition associated with CXCR4 expression comprises inhibiting the growth of cells that express CXCR4.

While not being limited to any particular theory, the mechanism of action can include, but is not limited to preventing SDF-1 binding to CXCR4, thereby inhibiting cell proliferation, adhesion and invasion.

In some embodiments of the disclosure, the animal to be treated is a human.

In some embodiments of the disclosure, the targeted binding agent is a fully human monoclonal antibody.

In some embodiments of the disclosure, the targeted binding agent is selected from the group consisting of fully human monoclonal antibodies 2A4, 4C1, 5C9, 5E1, 6C7 or 7C8 or an antibody comprising a VH and/or VL domain of any of the foregoing antibodies.

The targeted binding agent or antibody of the disclosure can be administered alone, or can be administered in combination with additional antibodies or chemotherapeutic drugs or radiation therapy. The target binding agent can be administered as part of a therapeutic regimen with, for example, surgery.

Any of the CXCR4 antibodies (or antigen binding fragments) of the disclosure can be used in any one or more of the foregoing methods. By way of example, any of the CXCR4 antibodies (or antigen binding fragments) of the disclosure having any one or more (1, 2, 3, 4, 5, 6, 7, 8, 9, etc.) of the functional and/or structural characteristics set forth herein can be used in any of the methods disclosed herein.

### (vi) Formulations

In certain embodiments, the CXCR4 antibodies (or antigen binding fragments) of the disclosure may be formulated with a pharmaceutically acceptable carrier as pharmaceutical compositions/preparations, and may be administered by a variety of methods known in the art. As will be appreciated by the skilled artisan, the route and/or mode of administration will vary depending upon the desired results. As used herein, the pharmaceutical formulations comprising the anti-CXCR4 antibodies of the disclosure are referred to as formulations (or preparations) of the disclosure. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also routinely contain compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the antibodies of the present disclosure, and with each other, in a manner such that there is no interaction which would substantially impair the desired pharmaceutical efficacy.

The formulations of the disclosure are present in a form known in the art and acceptable for therapeutic, diagnostic and/or research uses. In certain embodiments, a formulation of the disclosure is a liquid formulation. In another embodiment, a formulation of the disclosure is a lyophilized formulation. In a further embodiment, a formulation of the disclosure is a reconstituted liquid formulation. In another embodiment, a formulation of the disclosure is a stable liquid formulation. In another embodiment, a liquid formulation of the disclosure is an aqueous formulation. In another embodiment, the liquid formulation is non-aqueous. In another embodiment, a liquid formulation of the disclosure is an aqueous formulation wherein the aqueous carrier is distilled water.

The formulations of the disclosure comprise an anti-CXCR4 antibody of the disclosure in a concentration resulting in a w/v appropriate for a desired dose. In certain embodiments, the anti-CXCR4 antibody (or antigen binding fragment) is present in the formulation of the disclosure at a concentration of about 1 mg/ml to about 500 mg/ml.

Embodiments of the disclosure include sterile pharmaceutical formulations of anti-CXCR4 antibodies that are useful as treatments for diseases. In certain embodiments, such formulations would inhibit the binding of CXCR4 to its substrates, thereby treating pathological conditions where, for example, serum or tissue CXCR4 is abnormally elevated. Antibodies of the disclosure preferably possess adequate affinity to potently inhibit CXCR4 activity, or inhibit CXCR4 binding to its substrates. In certain embodiments, antibodies of the disclosure have an adequate duration of action to allow for infrequent dosing in humans. Additionally, the disclosure provides other formulations, including sterile formulations, in a suitable carrier suitable for use in vitro, in animal studies, and in diagnostics.

The route of antibody administration is in accord with known methods, e.g., injection or infusion by intravenous, intraperitoneal, intracerebral, intramuscular, intraocular, intraarterial, intrathecal, inhalation or intralesional routes, etc.

An effective amount of antibody to be employed therapeutically will depend, for example, upon the therapeutic objectives, the route of administration, and the condition of the patient.

Antibodies, as described herein, can be prepared in a mixture with a pharmaceutically acceptable carrier suitable for intended use (e.g., diagnostic, in vitro laboratory, therapeutic, etc.).

Sterile compositions for injection can be formulated according to conventional pharmaceutical practice as described in Remington: The Science and Practice of Pharmacy (20^{th} ed, Lippincott Williams & Wilkens Publishers (2003)).

The dosage of the antibody formulation for a given patient (human or animal) will be determined by the attending physician taking into consideration various factors known to modify the action of drugs including severity and type of disease, body weight, sex, diet, time and route of administration, other medications and other relevant clinical factors. Therapeutically effective dosages can be determined by either in vitro or in vivo methods. Moreover, appropriate dosages for other uses, such as diagnostic uses, can be similarly extrapolated from in vitro testing.

It will be appreciated that administration of therapeutic entities in accordance with the compositions and methods herein will be administered with suitable carriers, excipients, and other agents that are incorporated into formulations to provide improved transfer, delivery, tolerance, and the like.

### (vii) Articles of Manufacture and Kits

This section of the specification describes various exemplary kits and packages comprising anti-CXCR4 antibodies (including antigen binding fragments) of the present disclosure. It should be understood that any of the anti-CXCR4 antibodies or antigen binding fragments described herein, including antibodies or antigen binding fragments having any one or more of the structural and functional features described in detail throughout the application, may be packaged, sold, and/or used as part of a kit or package, as described in this section. When various kits and packages are described in this section as including an antibody, it is understood that such an antibody may be an antibody or an antigen binding fragment having any one or more of the characteristics of the anti-CXCR4 antibodies or antigen binding fragments described herein. The disclosure contemplates all combinations of any of the aspects and embodiments of the disclosure.

The disclosure provides a pharmaceutical package or kit comprising one or more containers filled with a liquid formulation or lyophilized formulation of the disclosure (e.g., a formulation comprising an anti-CXCR4 antibody or antigen binding fragment of the present disclosure). In certain embodiments, a container filled with a liquid formulation of the disclosure is a pre-filled syringe. In another embodiment, the formulations of the disclosure comprise anti-CXCR4 antibodies recombinantly fused or chemically conjugated to another moiety, including but not limited to, a heterologous protein, a heterologous polypeptide, a heterologous peptide, a large molecule, a small molecule, a marker sequence, a diagnostic or detectable agent, a therapeutic moiety, a drug moiety, a radioactive metal ion, a second antibody, and a solid support. In a specific embodiment, the formulations of the disclosure are formulated in single dose vials as a sterile liquid. The formulations of the disclosure may, for example, be supplied in 3 cc USP Type I borosilicate amber vials (West Pharmaceutical Serices—Part No. 6800-0675) with a target volume of 1.2 mL. Optionally associated with any such container(s) can be a notice in the form prescribed by a governmental agency regulating the manufacture, use or sale of pharmaceuticals or biological products, which notice reflects approval by the agency of manufacture, use or sale for human administration or veterinary administration. In another embodiment, a formulation of the disclosure may be supplied in a pre-filled syringe.

In certain embodiments, a container filled with a liquid formulation of the disclosure is a pre-filled syringe. Any pre-filled syringe known to one of skill in the art may be used in combination with a liquid formulation of the disclosure. Pre-filled syringes that may be used are described in, for example, but not limited to, WO05/032627, WO08/094984, WO99/45985, WO03/077976, U.S. Pat. No. 6,792,743, U.S. Pat. No. 5,607,400, U.S. Pat. No. 5,893,842, U.S. Pat. No. 7,081,107, U.S. Pat. No. 7,041,087, U.S. Pat. No. 5,989,227, U.S. Pat. No. 6,807,797, U.S. Pat. No. 6,142,976, U.S. Pat. No. 5,899,889, US 20070161961A1, US 20050075611A1, US 20070092487A1, US 20040267194A1, US 20060129108A1. Pre-filled syringes may be made of various materials. In one embodiment a pre-filled syringe is a glass syringe. In another embodiment, a pre-filled syringe is a plastic syringe. One of skill in the art understands that the nature and/or quality of the materials used for manufacturing the syringe may influence the stability of a protein formulation stored in the syringe. For example, it is understood that silicon based lubricants deposited on the inside surface of the syringe chamber may affect particle formation in the protein formulation. In one embodiment, a pre-filled syringe comprises a silicone based lubricant. In one embodiment, a pre-filled syringe comprises baked on silicone. In another embodiment, a pre-filled syringe is free from silicone based lubricants. One of skill in the art also understands that small amounts of contaminating elements leaching into the formulation from the syringe barrel, syringe tip cap, plunger or stopper may also influence stability of the formulation. For example, it is understood that tungsten introduced during the manufacturing process may adversely affect formulation stability. In one embodiment, a pre-filled syringe may comprise tungsten at a level above 500 ppb. In another embodiment, a pre-filled syringe is a low tungsten syringe. In another embodiment, a pre-filled syringe may comprise tungsten at a level between about 500 ppb and about 10 ppb, between about 400 ppb and about 10 ppb, between about 300 ppb and about 10 ppb, between about 200 ppb and about 10 ppb, between about 100 ppb and about 10 ppb, between about 50 ppb and about 10 ppb, between about 25 ppb and about 10 ppb.

In certain embodiments, kits comprising anti-CXCR4 antibodies are also provided that are useful for various purposes, e.g., research and diagnostic including for purification or immunoprecipitation of CXCR4 from cells, detection of CXCR4, etc. For isolation and purification of CXCR4, the kit may contain an anti-CXCR4 antibody coupled to beads (e.g., sepharose beads). Kits may be provided which contain the antibodies for detection and quantitation of CXCR4 in vitro, e.g. in an ELISA or a Western blot. As with the article of manufacture, the kit comprises a container and a label or package insert on or associated with the container. The container holds a composition comprising at least one anti-CXCR4 antibody (or antigen binding fragment) of the disclosure. Additional containers may be included that contain, e.g., diluents and buffers, control antibodies. The label or package insert may provide a description of the composition as well as instructions for the intended in vitro or diagnostic use.

The present disclosure also encompasses a finished packaged and labeled pharmaceutical product. This article of manufacture includes the appropriate unit dosage form in an appropriate vessel or container such as a glass vial, pre-filled syringe or other container that is hermetically sealed. In one embodiment, the unit dosage form is provided as a sterile particulate free solution comprising an anti-CXCR4 antibody that is suitable for parenteral administration. In another embodiment, the unit dosage form is provided as a sterile lyophilized powder comprising an anti-CXCR4 antibody that is suitable for reconstitution.

In certain embodiments, the unit dosage form is suitable for intravenous, intramuscular, intranasal, oral, topical or subcutaneous delivery. Thus, the disclosure encompasses sterile solutions suitable for each delivery route. The disclosure further encompasses sterile lyophilized powders that are suitable for reconstitution.

As with any pharmaceutical product, the packaging material and container are designed to protect the stability of the product during storage and shipment. Further, the products of the disclosure include instructions for use or other informational material that advise the physician, technician or patient on how to appropriately prevent or treat the disease or disorder in question, as well as how and how frequently to administer the pharmaceutical. In other words, the article of manufacture includes instruction means indicating or suggesting a dosing regimen including, but not limited to, actual doses, monitoring procedures, and other monitoring information.

In certain embodiments, the disclosure provides an article of manufacture comprising packaging material, such as a box, bottle, tube, vial, container, pre-filled syringe, sprayer, insufflator, intravenous (i.v.) bag, envelope and the like; and at least one unit dosage form of a pharmaceutical agent contained within said packaging material, wherein said pharmaceutical agent comprises a liquid formulation containing a CXCR4 antibody of the disclosure. The packaging material may include instruction means which indicate how the antibody can be used to prevent, treat and/or manage one or more symptoms associated with a disease or disorder.

### (VIII) Testing the Efficacy of Cxcr4 Antibodies

CXCR4 antibodies may be effective for treating a variety of diseases. The treatment efficacy of the CXCR4 antibodies may be evaluated in disease models which are well-known in the art. The efficacy of antibodies or antigen binding fragments of the disclosure may be evaluated in any one or more of the assays described below or otherwise known in the art. Exemplary assays and treatment regimens are summarized below.

i. Antiangiogenic efficacy

Antiangiogenic efficacy of CXCR4 antibodies (or fragments) of the disclosure may be assayed in a spheroid-based in vivo angiogenesis assay. In this assay, human umbilical vein endothelial cell (HUVEC) spheroids are prepared as described earlier (Korff and Augustin: J. Cell. Biol. 143: 1341-52, 1998) by pipetting 100 endothelial cells (EC) in a hanging drop on plastic dishes to allow overnight spheroid formation. The following day, using the method previously described (Alajati et al., Nature Methods 5:439-445, 2008), EC spheroids are harvested and mixed in a Matrigel/fibrin solution with single HUVECs to reach a final number of 100,000 ECs as spheroids and 200,000 single ECs per injected plug. VEGF-A and FGF-2 are added at a final concentration of 1000 ng/ml. Cohorts of 10 male SCID mice (5-8 weeks old) may be subcutaneously injected with 500 pl of the cell/matrix suspension. The following day (day 1), treatment may commence. In one embodiment, 6C7 antibody is dosed at 25 mg/kg two times per week. Vehicle only is used as control. At day 21 the study may be terminated. The matrix plugs are removed and fixed in 4% PFA. All matrix plugs are paraffin embedded and cut to a thickness of 8-10 μm for histological examination. Blood vessels are visualized and quantified by staining for human CD34, and pericyte coverage is determined by staining for smooth muscle actin (SMA).

ii. Ovarian Cancer

CXCR4 antibodies (or fragments) of the disclosure may also be tested for their ability to inhibit human tumor growth in SCID xenograft models of ovarian cancer. A human ovarian cancer line, such as HeyA8 or IGROV-1, is cultured at 37° C. in a CO₂ incubator in RPMI1640 media containing 10% Fetal Bovine Serum and 1% L-glutamine. 6-7 week old SCID female mice (Charles River Lab, Wilmington, Mass.) are injected subcutaneously with cells [E.g., IGROV-1 (3×10⁶ with 20% matrigel)] in FBS free RPMI 1640 media in a total volume of 100 μl into the right flank region. Tumors are allowed to grow to 100-200 mm³ and cohorts of 10 animals are randomized to control and treatment groups based on tumor size before the dosing is initiated. Tumor size is monitored by caliper measurement twice a week, and tumor volume is estimated using the formula volume=0.5XlengthXwidth². Antibody is administered intraperitoneally in a solution of sterile saline twice per week at the indicated doses.

To further explore the utility of CXCR4 antibodies in the treatment of tumors, antibodies may be administered in a xenograft model as above, except administered in preventative mode (dosing commenced one day after implantation of tumors).

This type of model may also be used to examine the effects of administering combinations of agents, as well as different doses, differing tumor size at commencement of treatment, etc. Moreover, different breast cancer cell lines can be used to test the efficacy in differ types of ovarian cancers.

iii. B Cell Lymphoma

CXCR4 antibodies (and fragments) of the disclosure may also be tested for ability to inhibit human tumor growth in SCID xenograft models of B-cell lymphoma. A human B-cell lymphoma line is cultured at 37° C. in a CO₂ incubator in RPMI1640 media containing 10% Fetal Bovine Serum and 1% L-glutamine. 1×10⁶ cells in 100 μl of serum free DMEM media are implanted subcutaneously into the right flank region of 6-7 week old SCID female mice (Charles River Lab, Wilmington, Mass.). Tumors are allowed to grow to 100-200 mm³ and cohorts of 10 animals are randomized to control and treatment groups based on tumor size before the dosing is initiated. Tumor size is monitored by caliper measurement twice a week, and tumor volume is estimated using the formula volume=0.5×length×width². Antibody is administered intraperitoneally in a solution of sterile saline twice per week at the indicated doses.

In a further exploration of the utility of CXCR4 antibodies of the disclosure in the treatment of tumors, antibodies described herein are administered in a subcutaneous xenograft model as above, except in preventative mode (dosing commenced one day after implantation of tumors).

This type of model may also be used to examine the effects of administering combinations of agents, as well as different doses, differing tumor size at commencement of treatment, etc. Moreover, different breast B cell lines can be used to test the efficacy in differ types of B cell cancers.

iv. Peripheral Blood Leukocytes

The effect of CXCR4 antibodies of the disclosure may also be assessed on peripheral blood leukocytes. CXCR4 is ubiquitously expressed on human peripheral blood leukocytes (PBLs). Thus, treatment with anti-CXCR4 antibodies runs the potential risk of affecting the function of leukocyte populations. To assess potential safety risks of CXCR4 inhibition in human leukocytes, human PBLs are isolated and treated ex vivo to determine effects of anti-CXCR4 antibodies on leukocyte populations.

Peripheral blood leukocytes are isolated from whole blood obtained fresh from normal donors. Whole blood is centrifuged to pellet cells, and red blood cells are lysed with ammonium chloride buffer. After several washes with PBS, PBLs are collected and resuspended in RPMI medium containing 10% human serum. Cells are plated at 100,000 cells/well in 96 well round bottom polystyrene plates, treated with 10 μg/mL antibody and incubated overnight (˜16-18 hours) at 37° C. in a 5% CO₂ incubator. Cells are stained with leukocyte markers (CD3, CD19, CD56) and samples are analyzed by flow cytometry (FACSCantoII), where a fixed volume is collected for each sample to determine absolute cell counts. Granulocyte, monocyte, and lymphocyte populations are separated based on forward and side scatter profile. Lymphocytes are further gated to separate B cells (CD19+), T cells (CD3+), and NK cells (CD56+).

In certain embodiments, this type of test is done as a counter-screen to assess potential safety risks of therapeutically effective antibodies.

v. Migration of HUVEC Cells

In other embodiments, the effect of CXCR4 inhibition on migration of HUVEC cells may be determined. A mechanism of action of a CXCR4 antibody may be inhibition of migration and mobility of endothelial precursor cells that may contribute to neoangiogenesis. As an experimental model of this, the ability of SDF-1 to stimulate migration of HUVEC cells in a scratch-wound healing experiment is tested, and subsequently the ability of CXCR4 antibodies to inhibit this migration is determined. HUVEC cells (Lonza) are plated in Human Endothlial Cell Growth Medium 2 (including supplements) and propagated up to passage 7. For the scratch-wound healing assay, cells are plated at 2×10⁵ cells/ml in Essen Imagelok 24 well plates in serum free or 2% serum endothelial cell growth medium (without additives), and cultured overnight. The medium is replaced with serum-free basal medium and cells cultured again overnight. The Essen scratch tool is used to produce scratch wounds in each well, released cells are washed with PBS, the medium is replaced by test media (basal medium+/−SDF-1, +/−antibodies), and places are cultured in the Incucyte system for further culture and imaging every 1 or 2 hrs. Images are analyzed with manufacturer's software to determine percent of wound healing (cells covering bare wound area).

### EXAMPLES

The examples below are given so as to illustrate the practice of this disclosure. They are not intended to limit or define the entire scope of this disclosure.

### Example 1

### Immunizations and Titering

### Immunogens

Chinese Hamster Ovary (CHO, American Type Tissue Collection, catalog # CCL-61) cells transiently transfected with human CXCR4, or Jurkat human T-cell leukemia cells, were used as immunogen for XenoMouse® immunizations. For the generation of the CHO transfectants, human full length CXCR4 cDNA (EMBL accession # M99293; Loetscher M, et al., J Biol Chem, 269:232-237, 1994) was inserted into pcDNA3.1 vector and lipofected into CHO cells. Expression of human CXCR4 at the cell surface at the level suitable for the purpose of immunization (30-50% transfection efficiency, geometric mean fluorescence ˜10-100 fold above background) was confirmed by fluorescent activated cell sorter (FACS) analysis. Batches of successfully transfected cells were frozen down and used as needed.

### Immunization

Monoclonal antibodies against CXCR4 were developed by immunizing ˜6 week old XenoMouse® mice [XenoMouse strains: XMG2 (IgG2 kappa/lambda) and XMG4 (IgG4 kappa/lambda) Amgen, Inc. Vancouver, British Columbia, Canada] with either one million Jurkat cells or CXCR4 transfected CHO cells. Groups of 10 XenoMouse animals were immunized via intraperitoneal and base of tail routes. Cells were suspended in PBS or aluminum phosphate gel adjuvant, HCL Biosector, (catalog #1452-250). Animals were boosted 3-6 days apart, for a total number of 11-17 boosts.

### Selection of Animals for Harvest by Titer

Titers of the antibodies against human CXCR4 were evaluated by testing for binding to human and mouse CXCR4 transiently expressed in HEK293T cells using a Fluorometric microvolume assay technology (FMAT) cellular detection instrument (Applied Biosystems). This analysis showed that there were some mice, primarily in the CHO immunization groups, that had significant titers of anti-CXCR4 specific antibody in their serum, as seen by comparison of FMAT signal on CXCR4 transfected HEK293T cells to the signal from parental HEK293T cells. Therefore, at the end of the immunization program, 8 mice were selected for harvest, and lymphocytes were isolated from the spleens and lymph nodes of the immunized mice, as described in example 2 below.

### Example 2

### Recovery of Lymphocytes, B-Cell Isolations, Fusions and Generation of Hybridomas

Immunized mice were sacrificed by cervical dislocation, and draining lymph nodes were harvested and pooled from each cohort. Spleens from four animals were also harvested and included for lymphocyte harvesting. The lymphoid cells were dissociated by grinding in DMEM to release the cells from the tissues, and the cells were suspended in DMEM. B cells were enriched by positive selection using CD19 labeled Dynal beads. A fusion was performed by mixing washed enriched B cells from above with non-secretory myeloma P3X63Ag8.653 cells (ATCC catalog # CRL 1580) (Kearney et al., J. Immunol. 123, 1979, 1548-1550) at a ratio of 1:1. The cell mixture was gently pelleted by centrifugation at 800×g. After complete removal of the supernatant, the cells were treated with 2-4 ml of Pronase solution (CalBiochem, catalog #53702; 0.5 mg/ml in PBS) for no more than 2 minutes. Then 3-5 ml of FBS was added to stop the enzyme activity and the suspension was adjusted to 40 ml total volume using electro cell fusion solution, ECFS (0.3 M sucrose, Sigma, catalog # S7903, 0.1 mM magnesium acetate, Sigma, catalog # M2545, 0.1 mM calcium acetate, Sigma, catalog # C4705). The supernatant was removed after centrifugation and the cells were resuspended in 40 ml ECFS. This wash step was repeated and the cells again were resuspended in ECFS to a concentration of 2×10⁶ cells/ml. Electro-cell fusion was performed using a fusion generator, model ECM2001, Genetronic, Inc., San Diego, Calif. The fusion chamber size used was 2.0 ml, using the following instrument settings: alignment condition: voltage: 50 V, time: 50 seconds; membrane breaking at: voltage: 3000 V, time: 30 μseconds; post-fusion holding time: 3 seconds. After ECF, the cell suspensions were carefully removed from the fusion chamber under sterile conditions and transferred into a sterile tube containing the same volume of Hybridoma Culture Medium (DMEM (JRH Biosciences), 15% FBS (Hyclone), supplemented with 2 mM L-glutamine (Sigma, catalog # G2150), 10 U/ml penicillin/0.1 mg/ml streptomycin (Sigma, catalog # P7539), 1 vial/L OPI (oxaloacetate, pyruvate, bovine insulin; Sigma catalog #05003) and 10 U/ml recombinant human IL-6 (Boehringer Mannheim, catalog #1131567). The cells were incubated for 15-30 minutes at 37° C., and then centrifuged at 400×g for 5 minutes. The cells were gently resuspended in a small volume of Hybridoma Selection Medium (Hybridoma Culture Medium supplemented with 0.5× HA (Sigma, catalog # A9666)), and the volume was adjusted appropriately with more Hybridoma Selection Medium, based on a final plating of 5×10⁶ B cells total per 96-well plate and 200 μl per well. The cells were mixed gently and pipetted into 96-well plates and allowed to grow. Exhaustive supernatants were collected from the cells that potentially produce anti-CXCR4 antibodies and subjected to subsequent screening assays as exemplified below.

### Example 3

### Binding to Human, Mouse and Cynomolgus Monkey CXCR4

Supernatants collected from harvested cells were tested to assess the ability of the secreted antibodies to bind to HEK293T cells transiently overexpressing either full-length human, murine or cynomolgus monkey CXCR4. A mock-transfected 293T cell line was used as a negative control. Cells diluted in PBS containing 2% FBS were seeded at a density of 3000 expressing and 15000 mock transfected cells per well in 384 well plates (Corning Costar, catalog #3712). Immediately after plating, 15 or 20 μl/well of hybridoma supernatants and 10 μl/well of secondary antibody (Goat anti-human IgG Fc Cy5, final concentration 750 ng/ml) were added and plates incubated for 3 hours at room temperature prior to reading the fluorescence on the FMAT 8200 instrument (Applied Biosystems). The product of number of positive events and fluorescence intensity was used as a measure of binding strength. Results for 6 hybridoma supernatants showing binding of hybridoma supernatants to human/cynomolgus monkey CXCR4 are shown in Table 2. All six monoclonal antibodies were positive for human and cynomolgus CXCR4 staining 5C9 showed general cellular background staining on mouse transfectants and was not considered positive. 6C7 showed substantial staining on mouse CXCR4 transfectants, and was considered a mouse CXCR4 positive antibody. However, further testing with mouse lymphocytes and the mouse B-cell line EL4 showed no reproducible staining on mouse CXCR4.

TABLE 2

| | Human CXCR4 | | | Cynomolgus CXCR4 | | | Mouse CXCR4 | | |
|---|---|---|---|---|---|---|---|---|---|
| | | | FL1 × | | | FL1 × | | | FL1 × |
| Ab ID | Count | FL1 | count | Count | FL1 | count | Count | FL1 | count |
| 5E1 | 213 | 7750 | 1650807 | 130 | 12899 | 1676888 | 86 | 1387 | 119258 |
| 6C7 | 199 | 7986 | 1589178 | 125 | 3474 | 434263 | 127 | 10641 | 1351380 |
| 7C8 | 184 | 7938 | 1460626 | 137 | 4684 | 641705 | 9 | 2396 | 21562 |
| 4C1 | 183 | 8326 | 1523626 | 46 | 6361 | 292620 | 5 | 1488 | 7439 |
| 2A4 | 89 | 8374 | 745320 | 89 | 6742 | 600052 | 3 | 954 | 2861 |
| 5C9 | 233 | 8799 | 2050253 | 106 | 7320 | 775869 | 72 | 4782 | 344287 |

Further investigation of cross-reactivity of antibody 6C7 to cynomolgus CXCR4 was conducted using the cynomolgous T-cell line HSC-F. In Kinexa-based affinity measurements (see Example 8) using the cynomolgus T-cell line HSC-F, 6C7 affinity for cynomolgus CXCR4 was estimated to be 221 pM. Functional activity of antibody 6C7 against cynomolgus CXCR4 was demonstrated in a chemotaxis assay using HSC-F cells stimulated with 125 nM SDF-1. Methods employed are described herein. Antibody 6C7 effectively inhibited migration of HSC-F cells with an IC50 comparable to the estimated affinity. 6C7 was also shown to inhibit signaling in the HSC-F cell line.

### Example 4

### Inhibition of Jurkat Chemotaxis and SDF-1 Binding

Supernatants collected from harvested cells were tested to assess the ability of the secreted antibodies to inhibit chemotaxis of Jurkat cells in response to SDF-1 stimulation. Jurkat cells were washed twice with serum free RPMI and resuspended in RPMI 1% BSA. Cells were incubated with test supernatants, desired dilutions of purified antibodies, or control antibodies, for 1 hr at 4° C. (2.5×10⁵cells/mL in RPMI+1% BSA) before transfer to the upper compartment of a 3 um HTS 96 well transwell membrane insert. SDF-1alpha (Peprotech) at 50 ng/ml in 100 uL of serum free RPMI+1% BSA was used in the lower chamber, and samples were incubated for 3.5 hours at 37° C. in 5% CO₂ incubator. At the end of the incubation, inserts were removed and migrated cells in the lower chamber were quantified by adding 25 uL CellTiterGlo (Promega), incubating for 10 minutes at room temp, transferring to a black plate and reading out luminescence per manufacturer's recommendation. This assay was repeated 3 times, and hybridomas that showed >60% inhibition of chemotaxis, were progressed for further testing.

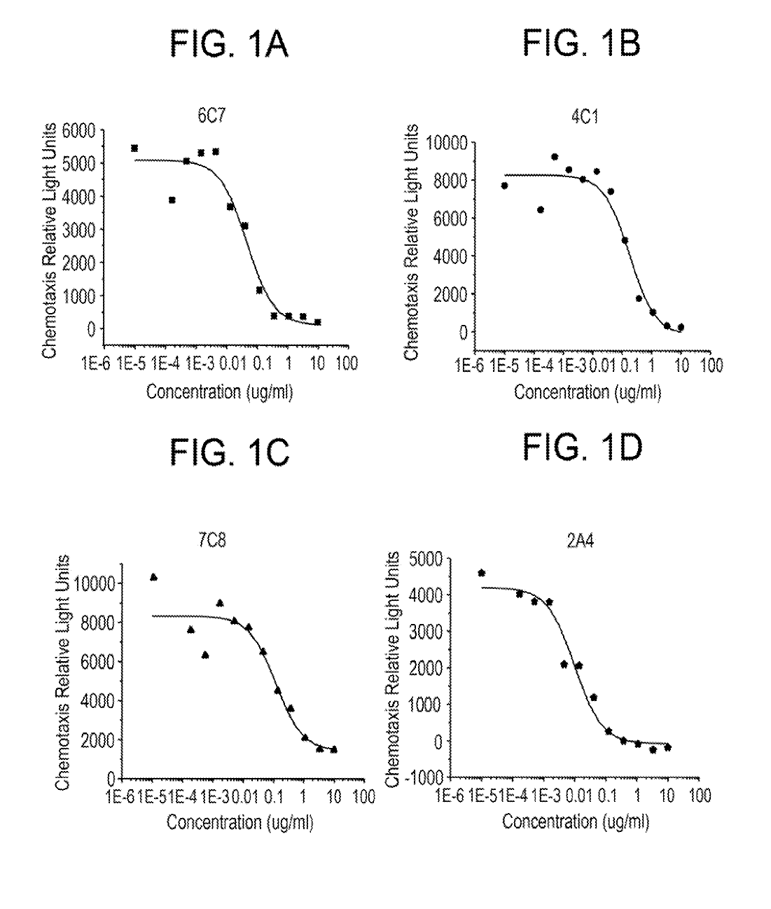

To investigate potential alternative mechanisms of action (e.g. downregulation, desensitization or internalization) of antibodies on CXCR4 receptor, the above chemotaxis inhibition assay was repeated using pre-incubation of Jurkat cells with antibody samples for 24 hours at 37° C. Average results of both the short-term and long-term chemotaxis assays are summarized in Table 3, and examples of chemotaxis curves are shown in FIG. 1.

Supernatants were further characterized by their ability to inhibit the binding of SDF-1 to human CXCR4 transfected HEK293T cells. SDF-1 at 1 mg/ml concentration was mixed with Alexa-647 labeling reagent starting with 625 nM. Working dilution of Alexa reagent was determined empirically, such that the labeled SDF-1 produced a 2-fold increase in geometric mean of fluorescence compared to background, when bound to CXCR4 expressing cells for 1 hour at 4° C. For determination of inhibition of SDF-1 binding, CXCR4 transfected HEK293T cells were pre-incubated with test supernatants and antibodies for 1 hour on ice, then Alexa labeled SDF-1 was added for another 1 hour on ice, washed 3 times, and fluorescence intensity read on a FACS Caliber.

Based on patterns of short and long term inhibition of chemotaxis, as well as inhibition of SDF-1 binding, a number of hybridomas were selected for limiting dilution subcloning, expansion, and purification.

TABLE 3
Inhibition of Jurkat chemotaxis and SDF-1 binding

| | % Inhibition | % Inhibition | % Inhibition of |
|---|---|---|---|
| Ab ID | 1 hr | 24 hr | SDF-1 binding |
| 5E1 | 70 | −20 | 1 |
| 6C7 | 99.6 | 95 | 88 |
| 7C8 | 48.3 | −34 | 75 |
| 4C1 | 81.3 | −27 | 35 |
| 2A4 | 98 | 76 | 42 |
| 5C9 | 74 | 44 | −1 |

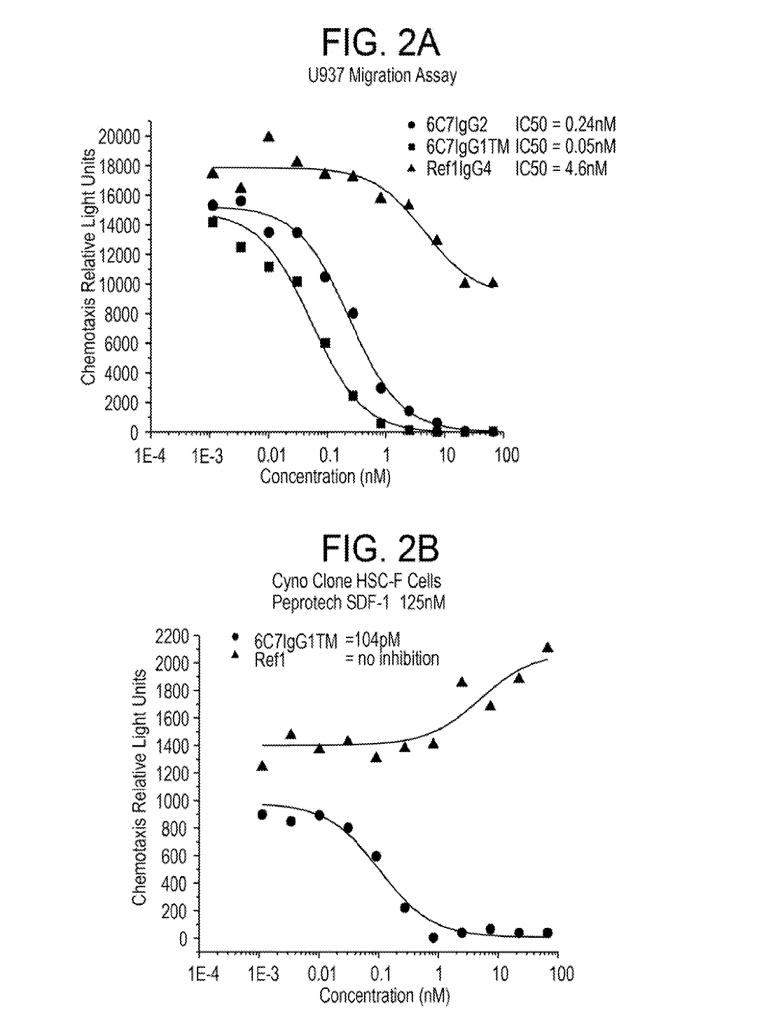

Purified monoclonal antibodies were further characterized based upon their potency in inhibition of SDF-1 induced chemotaxis of Jurkat cells. The 1-hour version of the chemotaxis experiment was performed as in Example 4 above, with the following modifications: RPMI medium with 1% heat-inactivated fetal bovine serum was used instead of BSA; and the concentration of SDF-1 was reduced to 25 ng/ml. In some experiments, the top transwells were transferred to a plate containing Versene for 10 minutes to detach loosely adherent cells at the bottom of the membrane, and CellTiterGlo signals from both parts of the sample were combined to obtain a total chemotaxis signal. The data were plotted against antibody concentration in OriginPro7 graphing software using the 4-parameter Pharmacology Dose Response function with the Hill slope set to 1. The IC50 values for each antibody as determined by the curve fit are summarized in Table 4, and representative dose-response curves are shown in FIG. 1. Comparable results from U937 line are shown in FIG. 2.

TABLE 4
JURKAT CHEMOTAXIS INHIBITION DOSE-RESPONSE

| | | | No. of |
|---|---|---|---|
| Ab ID | IC50 ng/ml | IC50 pM | experiments |
| 5E1 | 2125 | 14164 | 3 |
| 6C7 | 11.2 | 74.7 | 11 |
| 7C8 | 236 | 1574 | 2 |
| 4C1 | 182 | 1215 | 2 |
| 2A4 | 57.4 | 383 | 7 |
| 5C9 | 4220 | 28150 | 2 |

Additional experiments were conducted to determine the potency of CXCR4 antibodies in Jurkat chemotaxis inhibition. Antibody 6C7 was tested in both IgG2 and IgG1TM formats (heavy and light chain amino acid sequences for IgG1 TM format described above as 6C7-TM), while reference antibody Ref1 was tested in IgG4 and IgG1TM formats. Results from a number of experiments are summarized in Table 5:

TABLE 5
Summary of Jurkat Chemotaxis Inhibition

| | IC50 (nM) | # Experiments |
|---|---|---|
| 6C7 IgG2 | 0.068 | 20 |
| 6C7 IgG1TM | 0.193 | 18 |
| Ref1 IgG4 | 0.58 | 8 |
| Ref1 IgG1TM | 1.17 | 3 |

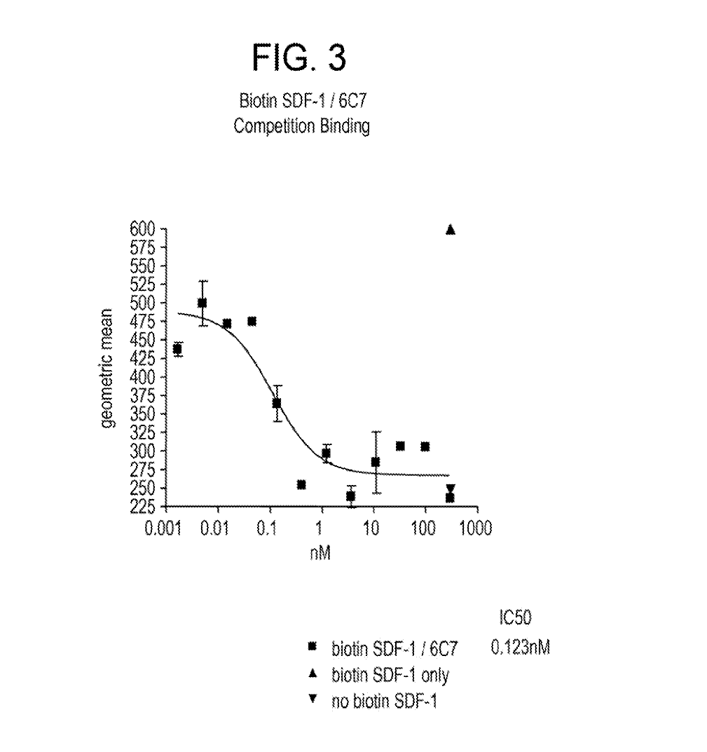

In addition to Jurkat cells, U937 lymphoma cells were also tested in a chemotaxis assay. Results of this experiment are shown in FIG. 2A. Antibody 6C7, in both isoforms, was able to inhibit migration of U937 cells completely, while reference antibody Ref1 in IgG4 format shows partial inhibition and lower potency in the concentration range tested. Similar experiments were conducted with the cynomolgus T-cell line HSC-F. 6C7 showed a dose-responsive inhibition of chemotaxis, with maximal inhibition near 100%, while the reference antibody Ref1 did not show consistent inhibition in this setting (FIG. 2B). Further experiments were conducted to investigate the ability of anti-CXCR4 antibodies to inhibit binding of SDF-1 to its receptor CXCR4 on Namalwa cells by FACS. In preliminary experiments it was determined that more consistent results could be obtained if the Namalwa cells were fixed with 1% buffered formalin for 10 minutes, and these conditions were used in the experiment. Fixed cells were incubated with 10 nM biotinylated SDF-1 for 15 min at 4° C., washed with PBS and subsequently incubated with various concentrations of CXCR4 antibody for 30 minutes at 4° C. After a second wash, cells were stained with 1 ug/ml streptavidin-PE and anlyzed by FACS on a FACS Caliber cytometer. As shown in FIG. 3, 6C7 was able to displace the binding of biotinylated SDF-1 with an IC50 comparable to its affinity to CXCR4.

### Example 5

### Inhibition of SDF-1 Driven CXCR4 Signaling

Antibodies were tested for their ability to inhibit CXCR4 mediated signaling, which is known to involve G-protein coupling, induction of Ca²⁺ release, and activation of MAP kinase and AKT pathways by phosphorylation. In preliminary experiments, it was observed that Jurkat cells show induction of phospho-MAP kinases (Erk1 and Erk2), but do not show significant induction of phospho-AKT. Jurkat cells were cultured in serum-free medium overnight prior to stimulation with 100 ng/ml of SDF-1. After 30 minutes, cells were lysed in PhosphoSafe buffer, loaded on Tris-Glycine gels, transferred to nitrocellulose, and probed with phospho-MAPK specific antibody. Antibodies 6C7 and 2A4, at 5 or 20 ug/ml, demonstrated inhibition of SDF-1 induced MAPK phosphorylation (near complete inhibition using above-described assay as representated by undetectable levels of phosphor-MAPK), while the other antibodies did not.

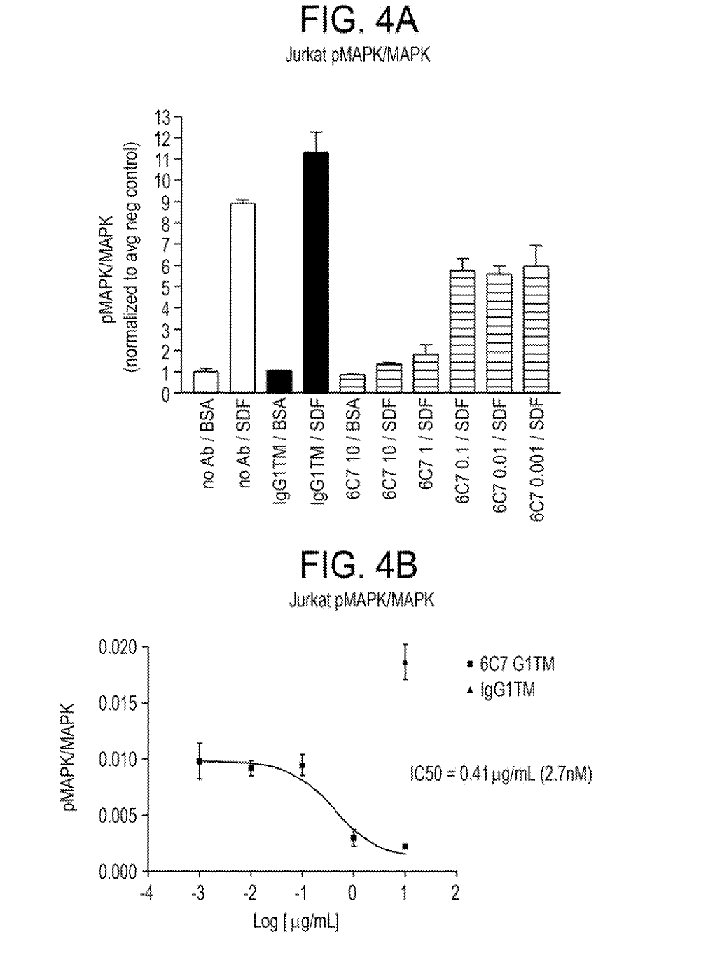

The effects of antibody treatment on SDF-1 induced MAPK phosphorylation were further assessed by quantitative western blot and ELISA. Jurkat cells were cultured in serum-free medium overnight, treated with anti-CXCR4 antibodies for 30 minutes on ice, then stimulated with 10 ng/mL SDF-1 for 30 minutes at 37° C. Phospho-MAPK levels were determined by western blot (as above), or by ELISA (Cell Signaling Technology). Western blot results exemplified in FIG. 4 show the SDF-1 stimulation window, as well as dose-responsive inhibition of pMAPK by 6C7. Furthermore, 6C7 antibody alone did not increase pMAPK levels, indicating that antibody treatment on its own does not result in an agonistic effect in this model. pMAPK ELISA results from a number of experiments showed that 6C7 inhibits SDF-1 driven MAPK phosphorylation in a dose dependent manner, with an IC50 value of ˜3 nM (see Table 6 for a summary).

TABLE 6
Inhibition of pMAPK Signaling by CXCR4 Antibodies

| | IC50 (nM) | St Dev | n |
|---|---|---|---|
| 6C7 IgG1TM | 3.1 | 1.5 | 9 |
| Ref1 IgG1TM | 13.0 | 5.7 | 4 |
| Ref1 IgG4 | 27.1 | 9.0 | 2 |

In similar experiments with MDA-MB-231 cells, a significant increase (˜2-fold) in phospho-AKT was detected upon stimulation with SDF-1, while there was no change in phospho-MAPK. When cells were treated with 10 ug/ml of 6C7 antibody, the SDF-1 induced phosphorylation was inhibited, as indicated by undectable levels of phopho-AKT. These results indicate that antibodies 6C7 and 2A4 inhibit SDF-1 mediated signaling through the CXCR4 receptor.

Phospho-AKT measurements were also carried out with the cynomolgus cell line HSC-F. As shown in FIG. 5, SDF-1 stimulation resulted in AKT phosphorylation in this setting. CXCR4 antibodies alone did not show any pAKT induction, confirming lack of agonist activity. 6C7 at 10 ug/ml resulted in inhibition of AKT phosphorylation, while the reference antibody Ref1 resulted in partial inhibition.

### Example 6

### Structural Analysis of Anti-CXCR4 Antibodies

cDNA clones encoding the heavy chain variable sequences and the variable light chains of the antibodies were sequenced. The nucleotide and amino acid sequences of variable heavy (VH) and variable light (VL) domains for the anti-CXCR4 antibodies are provided after the Examples section. The heavy chain variable domain sequences were analyzed to determine the VH gene segment, the D-gene and the JH-gene used by each variable domain. The sequences were then translated to determine the primary amino acid sequence of the variable domain as expressed in the lead antibody, and compared to the germline VH, D and J-region sequences to assess mutations from germline. Similarly, light chain variable domain sequences were analyzed to determine kappa or lambda V gene usage, and correspondingly Jk or JL gene usage. Translated expressed sequences were compared to germline sequences to assess mutations in relation to germline.

Tables 7 and 8 are tables comparing the antibody heavy chain regions to their cognate germ line heavy chain region and kappa light chain regions to their cognate germ line light chain region.

TABLE 7

| Heavy | | | | | | | | | | |
|---|---|---|---|---|---|---|---|---|---|---|
| chain | V | D | J | FRI | CDR1 | FR2 | CDR2 | FR3 | CDR3 | FR4 |
| 4C1 | VH 3-30 | D 6-25 | JH6 | QVQLVESGGGVVQPG | SYGMH | WVRQAPG | VISYDGSNK | RFTISRDNSKNTLYLQ | GGLAARRNY | WGQGTT |
| | | | | RSLRLSCAASGFTFS | | KGLEWVA | YYADSVKG | MNSLRAEDTAVYYCTR | YYSYGMDV | VTVSS |
| | Germline | | | QVQLVESGGGVVQPG | SYGMH | WVRQAPG | VISYDGSNK | RFTISRDNSKNTLYLQ | -GIAA--- | WGQGTT |
| | | | | RSLRLSCAASGFTFS | | KGLEWVA | YYADSVKG | MNSLRAEDTAVYYCAR | YYYYYGMDV | VTVSS |
| 6C7 | VH 3-30 | D 3-10 | JH6 | QVQLVESGGGVVQPG | NYVMH | WVRQAPG | VIWYDGSNK | RFTISRDNSKNTLSLQ | GEGYYGSGSR | WGQGTT |
| | | | | RSLRLSCAASGFTFS | | KGLEWVA | YYADSVKG | MNSLRAEDTAVYYCER | YRGYYYGMDV | VTVSS |
| | Germline | | | QVQLVESGGGVVQPG | SYGMH | WVRQAPG | VIWYDGSNK | RFTISRDNSKNTLYLQ | ---YYGSGSY | WGQGTT |
| | | | | RSLRLSCAASGFTFS | | KGLEWVA | YYADSVKG | MNSLRAEDTAVYYCAR | Y-YYYGMDV | VTVSS |
| 2A4 | VH 3-30 | D 6-25 | JH6 | QVQLVESGGGVVQPG | SYGMH | WVRQAPG | VISYDGSNK | RFTISRDNSKNTLYLQ | GGLAARRNYY | WGQGTT |
| | | | | RSLRLSCAASGFTFS | | KGLEWVA | YYADSVKG | MNSLRAEDTAVYYCTR | YSYGTDV | VTVSS |
| | Germline | | | QVQLVESGGGVVQPG | SYGMH | WVRQAPG | VISYDGSNK | RFTISRDNSKNTLYLQ | -GIAA---YY | WGQGTT |
| | | | | RSLRLSCAASGFTFS | | KGLEWVA | YYADSVKG | MNSLRAEDTAVYYCAR | YYYGMDV | VTVSS |
| 5C9 | VH 3-30 | D 2-21 | JH6 | QVQLVESGGGVVQPG | SYGLH | WVRQSPG | VISYDGSKK | RFSISRDNSKNTLYLQ | DRPSRYSS | WGQGTT |
| | | | | RSLRLSCAASGFTFS | | KGLEWVA | YYADSVKG | MNSLRAEDTAVYYCAR | CMDV | VTVSS |
| | Germline | | | QVQLVESGGGVVQPG | SYGMH | WVRQAPG | VISYDGSNK | RFTISRDNSKNTLYLQ | DCYS-YYY | WGQGTT |
| | | | | RSLRLSCAASGFTFS | | KGLEWVA | YYADSVKG | MNSLRAEDTAVYYCAR | GMDV | VTVSS |
| 5E1 | VH 3-23 | D 1-14 | JH6 | EVQLLESGGGLVQPG | SFAMN | WVRQAPG | AISGSGGNI | RFTISRDNSKNTLYLQ | VDRNLGYYH | WGQGTT |
| | | | | GSLRLSCAASGFTFS | | KGLEWVS | YYADSVRG | MNSLRAEDTAVYYCAK | GMDV | VTVSS |
| | Germline | | | EVQLLESGGGLVQPG | SYAMS | WVRQAPG | AISGSGGST | RFTISRDNSKNTLYLQ | --RN-YYY | WGQGTT |
| | | | | GSLRLSCAASGFTFS | | KGLEWVS | YYADSVKG | MNSLRAEDTAVYYCAK | GMDV | VTVSS |
| 7C8 | VH 3-33 | D 3-9 | JH4 | QVQLVESGGGVVQPG | SYGMH | WVRQAPG | VIWYDGTYK | RFTISRDNSKNTLYLQ | GPLLRYFDW | WGQGTL |
| | | | | RSLRLSCAASGFTFS | | KGLEWVA | YYASDVRG | MNSLRAEDTAVYYCAR | LSDY | VTVSS |
| | Germline | | | QVQLVESGGGVVQPG | SYGMH | WVRQAPG | VIWYDGSNK | RFTISRDNSKNTLYLQ | ---LRYFDW | WGQGTL |
| | | | | RSLRLSCAASGFTFS | | KGLEWVA | YYADSVKG | MNSLRAEDTAVYYCAR | L-DY | VTVSS |

TABLE 8

| Light | | | | | | | | | |
|---|---|---|---|---|---|---|---|---|---|
| Chain | V | J | FR1 | CDR1 | FR2 | CDR2 | FR3 | CDR3 | FR4 |
| 4C1 | VK A30 | JK4 | DIQMTQSPSSLS | RASQDIRN | WYQQKPGK | AASSLQS | GVPSRFSGSGSGTQFT | LQHNS | FGGGT |
| | | | ASVGDRVTITC | DLG | APKRLIY | | LTISSLQPEDFATYYC | YPLT | KVQIK |
| | Germline | | DIQMTQSPSSLS | RASQGIRN | WYQQKPGK | AASSLQS | GVPSRFSGSGSGTEFT | LQHSN | FGGGT |
| | | | ASVGDRVTITC | DLG | APKRLIY | | LTISSLQPEDFATYYC | YPLT | KVEIK |
| 6C7 | VK A30 | JK1 | DIQMTQSPSSLS | RASQGIRT | WYQQKPGK | AASSLQS | GVPSRFSGSGSGTEFT | LQHNS | FGQGT |
| | | | ASVGDRVTITC | DLG | APKRLIY | | LTISSLQPEDFATYYC | YPRT | KVEIK |
| | Germline | | DIQMTQSPSSLS | RASQGIRN | WYQQKPGK | AASSLQS | GVPSRFSGSGSGTEFT | LQHNS | FGQGT |
| | | | ASVGDRVTITC | DLG | APKRLIY | | LTISSLQPEDFATYYC | YPWT | KVEIK |
| 2A4 | VK A30 | JK4 | DIQMTQSPSSLS | RASQDIRN | WYQQKPGK | AASSLQS | GVPSRFSGSGSGTQFT | LQHNS | FGGGT |
| | | | ASVGDRVTITC | DLG | APTRLIY | | LTISSLQPEDFATYYC | YPLT | KVQIK |
| | Germline | | DIQMTQSPSSLS | RASQGIRN | WYQQKPGK | AASSLQS | GVPSRFSGSGSGTEFT | LQHNS | FGGGT |
| | | | ASVGDRVTITC | DLG | APKRLIY | | LTISSLQPEDFATYYC | YPLT | KVEIK |
| 5C9 | VL 2b2 | JL2 | QSALTQPASVS | TGTSSDVG | WYQQHPGK | EVSKRPS | GVSNRFSGSKSGNTAS | CSYAGS | FGGGT |
| | | | GSPGQSITISC | SNNFVS | APKLMIY | | LTISGLQAEDEADYYC | NTL-V | KLTVL |
| | Germline | | QSALTQPASVS | TGTSSDVG | WYQQHPGK | EVSKRPS | GVSNRFSGSKSGNTAS | CSYAGS | FGGGT |
| | | | GSPGQSITISC | SYNLVS | APKLMIY | | LTISGLQAEDEADYYC | STFVV | KLTVL |
| 5E1 | VK A3/A19 | JK3 | DIVMTQSPLSLP | RSSQSLLH | WYLQKPGQ | LGSNRAS | GVPDRFSGSGSGSDFT | MQAL | FGPGT |
| | | | VTPGEPASISC | SNGYNYLD | SPQLLIY | | LKISRVEAEDVGVYYC | Q-FT | KVDIK |
| | Germline | | DIVMTQSPLSLP | RSSQSLLH | WYLQKPGQ | LGSNRAS | GVPDRFSGSGSGTDFT | MQALQ | FGPGT |
| | | | VTPGEPASISC | SNGYNYLD | SPQLLIY | | LKISRVEAEDVGVYYC | TPFT | KVDIK |
| 7C8 | VK A30 | JK4 | DIQMTQSPSSLS | RASQGIRN | WYQQKPGK | AATSLQS | GVPSRFSGSGSGTEFT | LQHNN | FGGGT |
| | | | ASVGDRVTITC | DLG | APKRLIY | | LTISSLQPEDFATYYC | YPRT | KVEIK |
| | Germline | | DIQMTQSPSSLS | RASQGIRN | WYQQKPGK | AASSLQS | GVPSRFSGSGSGTEFT | LQHNS | FGGGT |
| | | | ASVGDRVTITC | DLG | APKRLIY | | LTISSLQPEDFATYYC | YPLT | KVEIK |

The variable (V) regions of immunoglobulin chains are encoded by multiple germ line DNA segments, which are joined into functional variable regions (V_{H}DJ_{H}, V_{K}J_{K} or V_{L}J_{L}) during B-cell ontogeny. The molecular and genetic diversity of the antibody response to CXCR4 was studied in detail.

It should also be appreciated that where a particular antibody differs from its respective germline sequence at the amino acid level, the antibody sequence can be mutated back to the germline sequence. Such corrective mutations can occur at one, two, three or more positions, or a combination of any of the mutated positions, using standard molecular biological techniques. By way of non-limiting example, the heavy chain of 4C1 differs from the corresponding germline sequence at amino acid 97 (see Table 10) by a T to an A. Thus the heavy chain amino acid sequence can be modified such that it now incorporates at amino acid 97 an A at position 97. Tables 9-11 below illustrate the positions of such variations from the germline for mAb 4C1, 2A4, and 6C7. Each row represents a unique combination of germline and non-germline residues at the position indicated by bold type.

In another embodiment, the disclosure includes replacing any structural liabilities in the sequence that might affect the heterogeneity of the antibodies of the disclosure. Such liabilities include glycosylation sites, un-paired cysteines, surface exposed methinones, etc. To reduce the risk of such heterogeneity it is proposed that changes are made to remove one or more of such structural liabilities.

TABLE 9
Exemplary Mutations of 4C1 light Chain (SEQ ID NO:
4) to Germline at the Indicated Residue Number

| 70 | 105 |
|---|---|
| Q | Q |
| E | Q |
| Q | E |
| E | E |

In some embodiments of the disclosure, the antibody comprises a sequence comprising SEQ ID NO: 4. In certain embodiments, SEQ ID NO: 4 comprises any one of the combinations of germline and non-germline residues indicated by each row of Table 9. In some embodiments, SEQ ID NO: 4 comprises any one, any two, or all two of the germline residues as indicated in Table 9. In certain embodiments, SEQ ID NO: 4 comprises any one of the unique combinations of germline and non-germline residues indicated by each row of Table 9. In other embodiments, the antibody is derived from a germline sequence with VK A30J and JK4 domains, wherein one or more residues has been mutated to yield the corresponding germline residue at that position.

TABLE 10
Exemplary Mutations of 6C7 Heavy Chain (SEQ ID NO:
6) to Germline at the Indicated Residue Number

| 31 | 33 | 80 | 97 |
|---|---|---|---|
| N | V | S | E |
| S | V | S | E |
| N | G | S | E |
| S | G | S | E |
| N | V | Y | E |
| S | V | Y | E |
| N | G | Y | E |
| S | G | Y | E |
| N | V | S | A |
| S | V | S | A |
| N | G | S | A |
| S | G | S | A |
| N | V | Y | A |
| S | V | Y | A |
| N | G | Y | A |
| S | G | Y | A |

In some embodiments of the disclosure, the antibody comprises a sequence comprising SEQ ID NO: 6. In certain embodiments, SEQ ID NO: 6 comprises any one of the combinations of germline and non-germline residues indicated by each row of Table 10. In some embodiments, SEQ ID NO: 6 comprises any one, any two, any three, any four, or all four of the germline residues as indicated in Table 10. In certain embodiments, SEQ ID NO: 6 comprises any one of the unique combinations of germline and non-germline residues indicated by each row of Table 10. In other embodiments, the antibody is derived from a germline sequence with VH3-33, D3-10 and JH6 domains, wherein one or more residues has been mutated to yield the corresponding germline residue at that position. In one specific example, the SEQ ID NO: 6 is modified back to germline sequence at position 80 by mutating a Y to an S and at position 97 by mutating an A to an E.

TABLE 11
Exemplary Mutations of 6C7 light Chain (SEQ ID NO:
8) to Germline at the Indicated Residue Number

| 31 | 96 |
|---|---|
| T | R |
| N | R |
| T | W |
| N | W |

In some embodiments of the disclosure, the antibody comprises a sequence comprising SEQ ID NO: 8. In certain embodiments, SEQ ID NO: 8 comprises any one of the combinations of germline and non-germline residues indicated by each row of Table 11. In some embodiments, SEQ ID NO: 8 comprises any one, any two, or all two of the germline residues as indicated in Table 11. In certain embodiments, SEQ ID NO.: 8 comprises any one of the unique combinations of germline and non-germline residues indicated by each row of Table 11. In other embodiments, the antibody is derived from a germline sequence with VK, A30 and JK1 domains, wherein one or more residues has been mutated to yield the corresponding germline residue at that position.

TABLE 12
Exemplary Mutations of 2A4 light Chain (SEQ ID NO:
12) to Germline at the Indicated Residue Number

| 45 | 70 | 105 |
|---|---|---|
| T | Q | Q |
| K | Q | Q |
| T | E | Q |
| K | E | Q |
| T | Q | E |
| K | Q | E |
| T | E | E |
| K | E | E |

In some embodiments of the disclosure, the antibody comprises a sequence comprising SEQ ID NO: 12. In certain embodiments, SEQ ID NO: 12 comprises any one of the combinations of germline and non-germline residues indicated by each row of Table 12. In some embodiments, SEQ ID NO: 12 comprises any one, any two, any three, or all three of the germline residues as indicated in Table 12. In certain embodiments, SEQ ID NO: 12 comprises any one of the unique combinations of germline and non-germline residues indicated by each row of Table 11. In other embodiments, the antibody is derived from a germline sequence with VK, A30 and JK4 domains, wherein one or more residues has been mutated to yield the corresponding germline residue at that position.

### Example 7

### Selectivity of CXCR4 Antibodies

CXCR4 is a GPCR, with few close homologues in the family of human GPCRs. Among these are CXCR3 and CCR4. To ensure that CXCR4 antibodies of the disclosure were selective to CXCR4, binding to human CXCR3 transfected HEK293 cells and CCR4 transfected CHO cells was investigated. Purified 2A4, 4C1, 6C7 and 7C8 antibodies were tested at 10 and 100 ug/ml concentration, and geometric mean fluorescence intensity was determined by FACS analysis, using parental cell lines as controls. Cells were incubated with primary antibodies and corresponding isotype controls for 1 hour on ice, followed by incubation with 1:50 Donkey anti Human IgG-FITC (Jackson 709 095 149). The samples were analyzed on a FACS Caliber cytometer. No staining above background was observed with any of the samples tested.

### Example 8

### Determination of Binding Affinity of Purified Antibodies

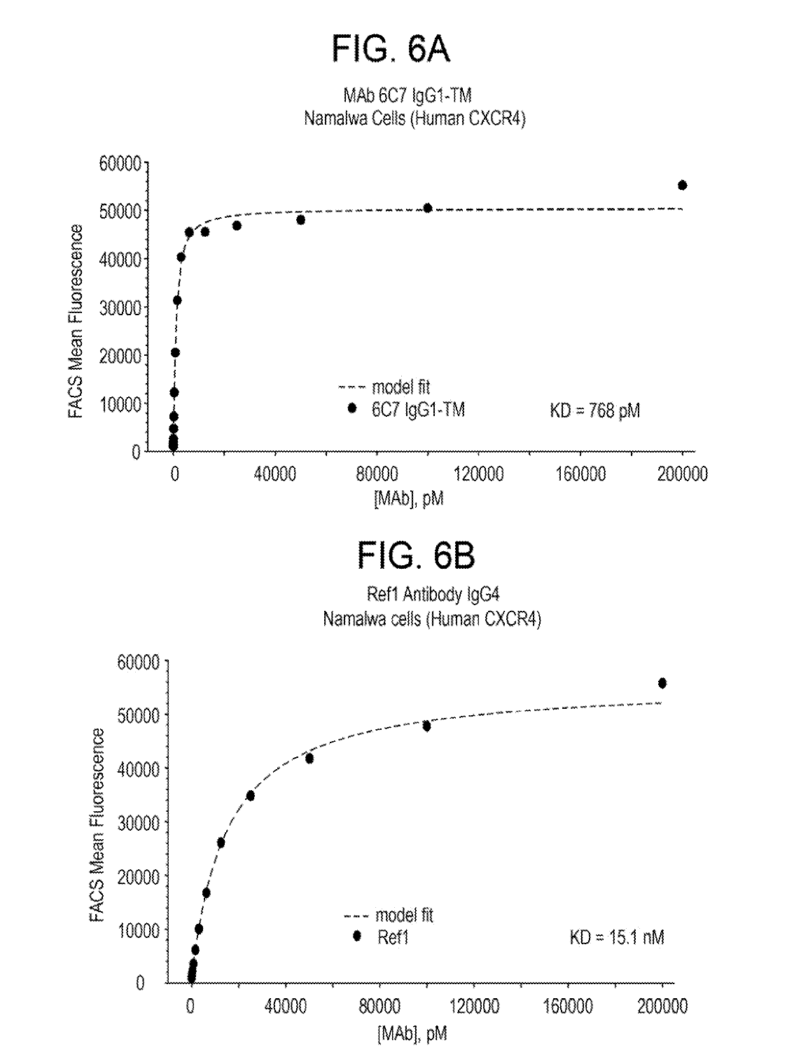

FACS binding under antigen limiting conditions was utilized to estimate the affinities of antibodies to the CXCR4 receptor. Jurkat cells were washed in RPMI1640 medium and plated at 50,000 cells per well in duplicate for incubation with 4-fold dilution series of antibody in duplicate, ranging between 150,000 and 0.143 pM. Incubation was carried out at 4° C. overnight, followed by wash step (3 times in PBS) and staining with Goat anti-human Fc−Cy5 secondary antibody+5 μg/mL 7-Amino-Actinomycin (7AAD) for 40 minutes at 4° C. Samples were analyzed on a FACSCaliber following another round of 3 washes in PBS. The geometric mean fluorescence intensity (Geo mean) of 5000 cell events was used for estimating the affinity. The negative geo mean was entered into Kinexa analysis software as an estimate of antibody “depletion” under limited antigen conditions, and the equilibrium dissociation constant (K_{D}) was estimated from the curve fit. These estimates are shown in Table 13. Note that 6C7 affinity measurements in two different experiments using different batches of antibody produced somewhat different results, and may reflect limitations of the Kinexa method to discriminate in the sub-nanomolar affinity range. Representative results for 6C7, along with reference antibody Ref1, are shown in FIG. 6.

TABLE 13
FACS Estimate of Affinity of antibodies to human CXCR4

| Antibody ID | KD (pM) | Range (pM) |
|---|---|---|
| 5E1 | 45 | 24-68 |
| 6C7 | 242 (Expt 1) | 148-367 |
| | 768 (Expt 2) | 481-1060 |
| 7C8 | 363 | 266-495 |
| 4C1 | 2200 | 1420-3320 |
| 2A4 | 820 | 502-1130 |
| 5C9 | Curve fit failed - may have | |
| | two affinities | |

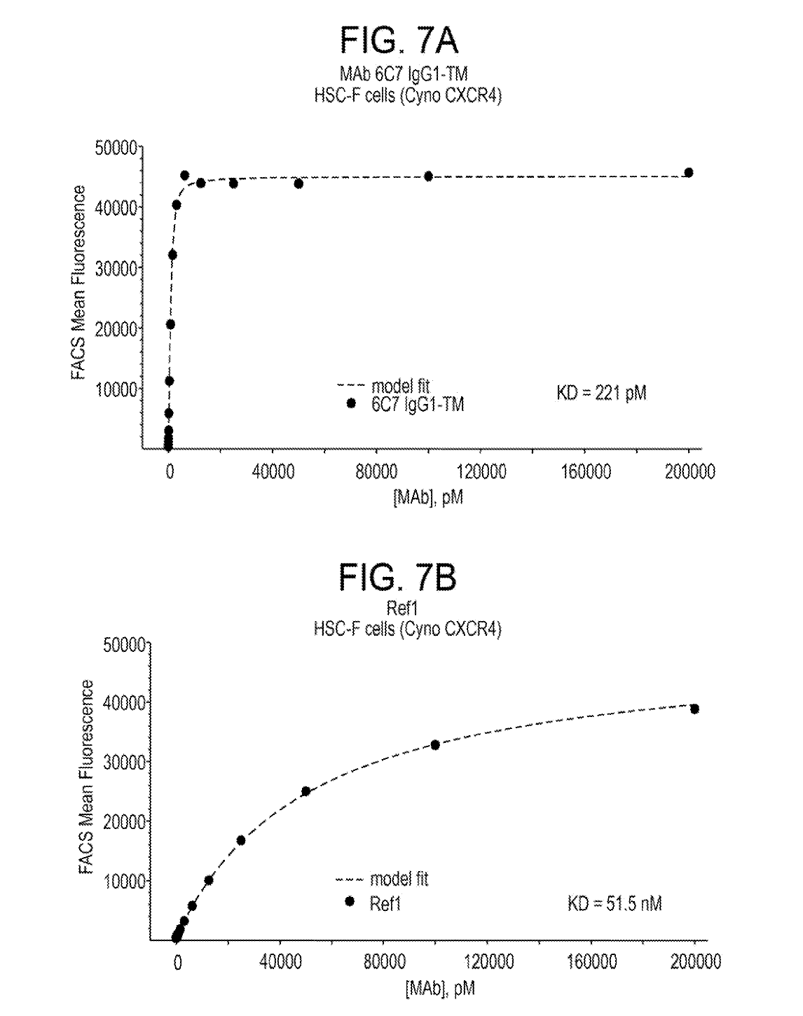

Similar experiments were conducted using the cynomolgus monkey T-cell line HSC-F. 6C7 again demonstrated subnanomolar binding affinity to cynomolgus CXCR4. The affinity model fitting for Kinexa data are shown in FIG. 7.

### Example 9

### Induction of Apoptosis by Anti-CXCR4 Antibodies

Anti-CXCR4 antibodies were tested for their ability to induce apoptosis in the human B-cell lymphoma line RAMOS. The cells were cultured in RPMI1640 with 10% heat inactivated fetal bovine serum and 2 mM L-glutamine at 37° C. in 5% CO₂. Purified antibodies were added to cultured cells at a final concentration of 10 ug/ml. After further incubation overnight, cells were washed and stained for Annexin V expression and viability (using ToPro iodide). Cells were analyzed by FACS on a FACSCaliber. ToPro positive cells were generally also positive for Annexin V, which was therefore used as a more sensitive measure of apoptosis induction. Untreated cells showed 5-10% staining with Annexin V. Treatment with antibody 6C7 resulted in 30-60% induction of apoptosis, while treatment with 2A4 antibody resulted in 20-40% apoptosis in independent experiments. In a repeat experiment evaluated at 72 hr, ˜50% induction of apoptosis was observed with antibody 6C7, with varying degrees of apoptosis with other lead antibodies under evaluation. These results are shown in FIG. 8.

### Example 10

### Evaluation of the Antiangiogenic Efficacy of CXCR4 Antibodies in a Spheroid-Based In Vivo Angiogenesis Assay

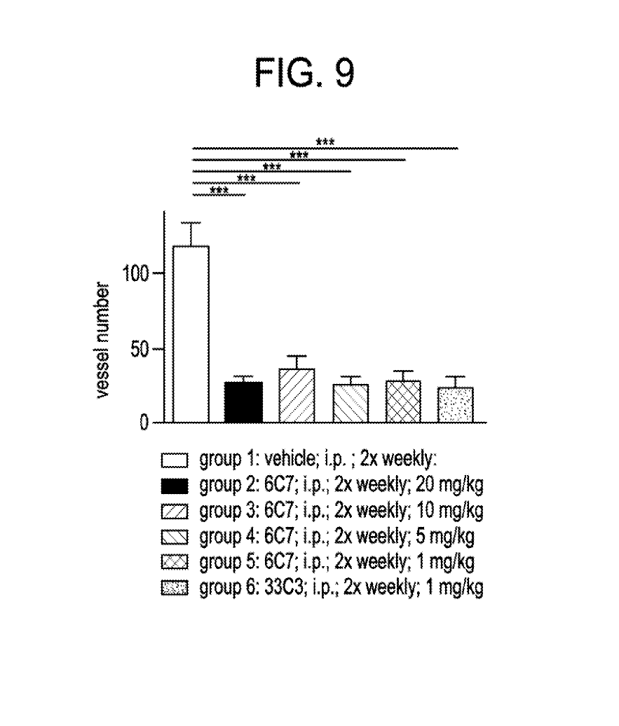

Human umbilical vein endothelial cell (HUVEC) spheroids were prepared as described earlier (Korff and Augustin: J. Cell. Biol. 143: 1341-52, 1998) by pipetting 100 endothelial cells (EC) in a hanging drop on plastic dishes to allow overnight spheroid formation. The following day, using the method previously described (Alajati et al., Nature Methods 5:439-445, 2008), EC spheroids were harvested and mixed in a Matrigel/fibrin solution with single HUVECs to reach a final number of 100,000 ECs as spheroids and 200,000 single ECs per injected plug. VEGF-A and FGF-2 were added at a final concentration of 1000 ng/ml. Cohorts of 10 male SCID mice (5-8 weeks old) were subcutaneously injected with 500 μl of the cell/matrix suspension. The following day (day 1) treatment commenced. 6C7 antibody was dosed at 25 mg/kg two times per week. Vehicle only was used as control. At day 21 the study was terminated. The matrix plugs were removed and fixed in 4% PFA. All matrix plugs were paraffin embedded and cut to a thickness of 8-10 μm for histological examination. Blood vessels were visualized and quantified by staining for human CD34, and pericyte coverage was determined by staining for smooth muscle actin (SMA). The data obtained suggest that treatment with 6C7 anti-CXCR4 antibody substantially inhibited (˜80%) human vessel formation compared to untreated control, but did not impact pericyte coverage (as assessed by the percentage of human CD34 positive vessels that were also associated with cells positive for αSMA expression). The data indicated that the antibody is active in an in vivo assay of angiogenesis. In a subsequent series of experiments, the dose response of inhibition of vessel formation was investigated. Maximal inhibition was observed between 20 and 1 mg/kg antibody treatment (twice weekly). This level was comparable to inhibition by the control antibody 33C3 that blocks mouse KDR. These results are shown in FIG. 9.

### Example 11

### Efficacy in Ovarian Cancer Xenograft Model

Anti-CXCR4 antibody 6C7 was also tested for its ability to inhibit human tumor growth in Nude xenograft models of ovarian cancer. The human ovarian cancer line HeyA8Luc+Clone 4 was cultured at 37° C. in a CO₂ incubator in RPMI1640 media containing 10% Fetal Bovine Serum and 1% L-glutamine, and the results from these experiments are described in detail below. A similar set of experiments was also performed using a different human ovarian cancer line: IGROV-1. However, given inconsistencies in the results obtained across multiple experiments using this line, we deemed these experiments and their data inconclusive, and thus, not suitable for inclusion herein.

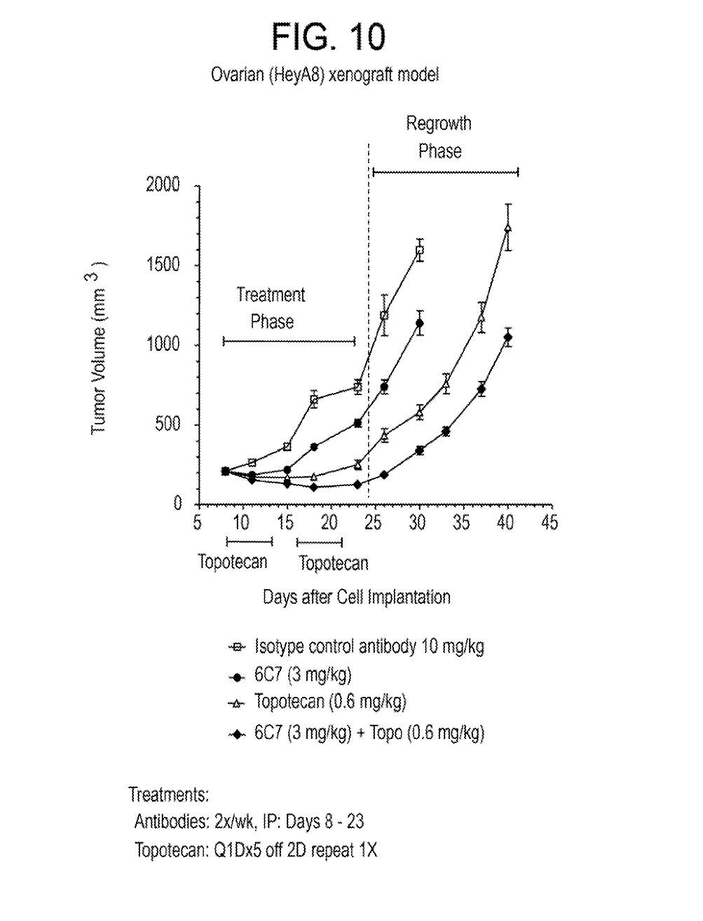

4-6 week old Nude female mice (Harlan Sprague Dawley, Indianapolis, Ind.) were injected subcutaneously with HeyA8Luc+Clone 4 (5×10⁶ with 50% matrigel in PBS) in a total volume of 200 μl into the right flank region. Tumors were allowed to grow to 190-250 mm³ and cohorts of 10 animals were randomized to control and treatment groups based on tumor size before the dosing was initiated. Tumor size was monitored by caliper measurement twice a week, and tumor volume was estimated using the formula volume=0.5×length×width². Antibody was administered intraperitoneally in a solution of sterile PBS twice per week at the indicated doses. As shown in FIG. 10, treatment of established tumors with 6C7 antibody resulted in a reduction in tumor growth (˜37%). In a further exploration of the potential utility of CXCR4 antibody in the treatment of tumors, 6C7 antibody was administered in combination with topotecan (0.6 mg/kg) in the xenograft model above. Antibody 6C7 showed improved TGI in combination with topotecan, resulting in ˜81% inhibition at 3 mg/kg dose (FIG. 10). Furthermore, the activity of 6C7 was dose dependent, at 10 mg/kg dose showing maximal activity. In addition, similar results of improved TGI, resulting in ˜72% inhibition, were observed when 6C7 was combined with doxorubicin in this ovarian xenograft.

### Example 12

### Effects of Cxcr4 Antibody Treatment on Peripheral Blood Leukocytes

CXCR4 is ubiquitously expressed on human peripheral blood leukocytes (PBLs). Thus, treatment with anti-CXCR4 antibody runs the potential risk of affecting the function of leukocyte populations. To assess potential safety risks of CXCR4 inhibition in human leukocytes, human PBLs were isolated and treated ex vivo to determine effects of anti-CXCR4 antibodies on leukocyte populations.

Peripheral blood leukocytes were isolated from whole blood obtained fresh from normal donors. Whole blood was centrifuged to pellet cells, and red blood cells were lysed with ammonium chloride buffer. After several washes with PBS, PBLs were collected and resuspended in RPMI medium containing 10% human serum. Cells were plated at 100,000 cells/well in 96 well round bottom polystyrene plates, treated with 10 μg/mL antibody and incubated overnight (˜16-18 hours) at 37° C. in a 5% CO₂ incubator. Cells were stained with leukocyte markers (CD3, CD19, CD56) and samples were analyzed by flow cytometry (FACSCantoII), where a fixed volume was collected for each sample to determine absolute cell counts. Granulocyte, monocyte, and lymphocyte populations were separated based on forward and side scatter profile. Lymphocytes were further gated to separate B cells (CD19+), T cells (CD3+), and NK cells (CD56+).

No significant changes were observed in granulocyte, monocyte, T cell, or NK cell populations with anti-CXCR4 antibody treatment compared to untreated control (data not shown). B cell loss was observed with anti-CXCR4 treatment vs. untreated control (see Table 14). This observation is consistent with reported activity of SDF-1 as a B-cell survival factor. Note that 6C7 treatment resulted in an ˜50% decrease in B-cell counts, while treatment with the reference antibody Ref1, in either IgG1TM or IgG4 formats, reduced B-cell counts by ˜80%.

TABLE 14

| | B-cell counts normalized |
|---|---|
| Treatment | to untreated control +/− SD |
| Untreated | 100.0 +/− 3.7 (n = 4) |
| IgG1TM control | 93.2 +/− 15.3 (n = 3) |
| IgG4 control | 102.3 +/− 9.2 (n = 2) |
| 6C7 IgG1TM | 49.5 +/− 6.7 (n = 4) |
| Ref1 IgG1TM | 19.9 +/− 3.0 (n = 3) |
| Ref1 IgG4 | 18.3 +/− 7.4 (n = 2) |

### Example 13

### Effect of CXCR4 Inhibition on Migration of Huvec Cells

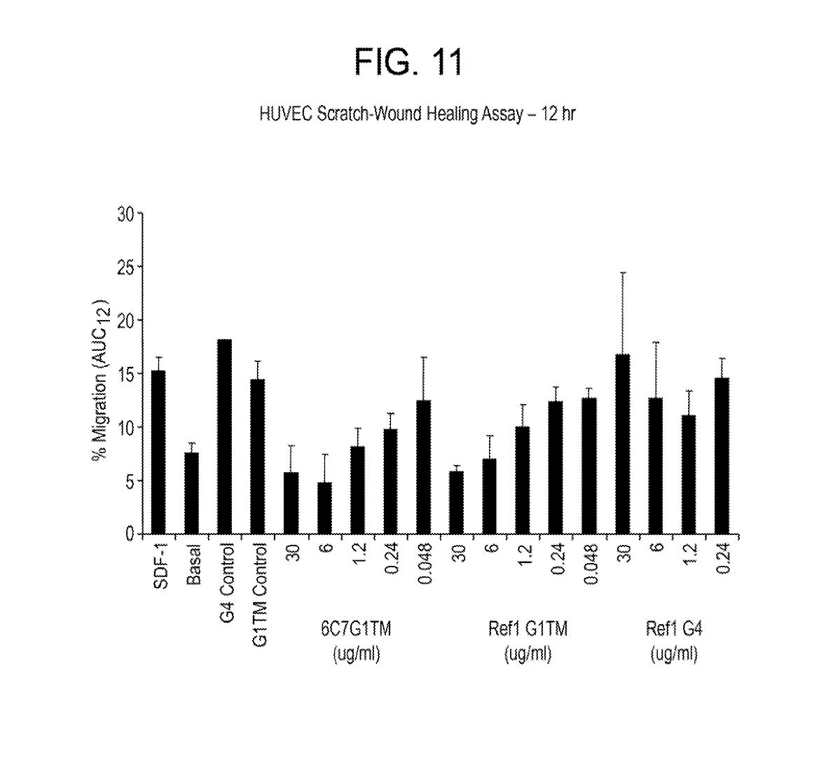

Another mechanism of action of a CXCR4 antibody may be inhibition of migration and mobility of endothelial precursor cells that may contribute to neoangiogenesis. As an experimental model of this, we tested ability of SDF-1 to stimulate migration of HUVEC cells in a scratch-wound healing experiment, and subsequently the ability of CXCR4 antibodies to inhibit this migration. HUVEC cells (Lonza) were plated in Human Endothlial Cell Growth Medium 2 (including supplements) and propagated up to passage 7. For the scratch-wound healing assay, cells were plated at 2×10⁵ cells/ml in Essen Imagelok 24 well plates in serum free or 2% serum endothelial cell growth medium (without additives), and cultured overnight. The medium was replaced with serum-free basal medium and cells cultured again overnight. The Essen scratch tool was used to produce scratch wounds in each well. Released cells were washed with PBS, the medium was replaced by test media (basal medium+/−SDF-1, +/−antibodies), and plates were cultured in the Incucyte system for further culture and imaging every 1 or 2 hours. Images were analyzed with manufacturer's software to determine percent of would healing (cells covering bare wound area). Representative results from one of three experiments are shown in FIG. 11. SDF-1 stimulated HUVEC migration above basal levels, and 6C7 IgG1TM antibody treatment suppressed this migration, sometimes below the basal level, in a dose dependent manner. Depending on the experiment, the reference antibody Ref1 produces comparable or lower inhibition of wound healing in IgG1TM format, but showed minimal or no activity in IgG4 format.

### Example 14

### Multiple Myeloma Model

Luciferase transfected MM1.S cells (a multiple myeloma cell line) were cultured in RPMI1640 media supplemented with 10% FBS, 2 mM L-glutamine, and 250 ug/ml G418. Twenty million cells were implanted intravenously via the tail vein into each female CB-17 SCID mouse. On day 21, the tumor burden was assessed using the Xenogen IVIS 100 imaging system. Mice were imaged in the dorsal and ventral positions and the bioluminescence signal is determined using Xenogen Living Image software. Dorsal and ventral values were added together for a total whole body bioluminescence. Mice were randomized into treatment groups based on the total whole body bioluminescence value. Mice were treated with vehicle control, negative antibody control, 6C7 and/or VELCADE twice weekly until mice began to show humane endpoints, usually hind limb paralysis. Tumor burden was monitored by bioluminescence imaging as above, spaced 4-7 days apart. As mice exhibited humane endpoints, they were euthanized. Both tumor burden and survival were evaluated as endpoints.

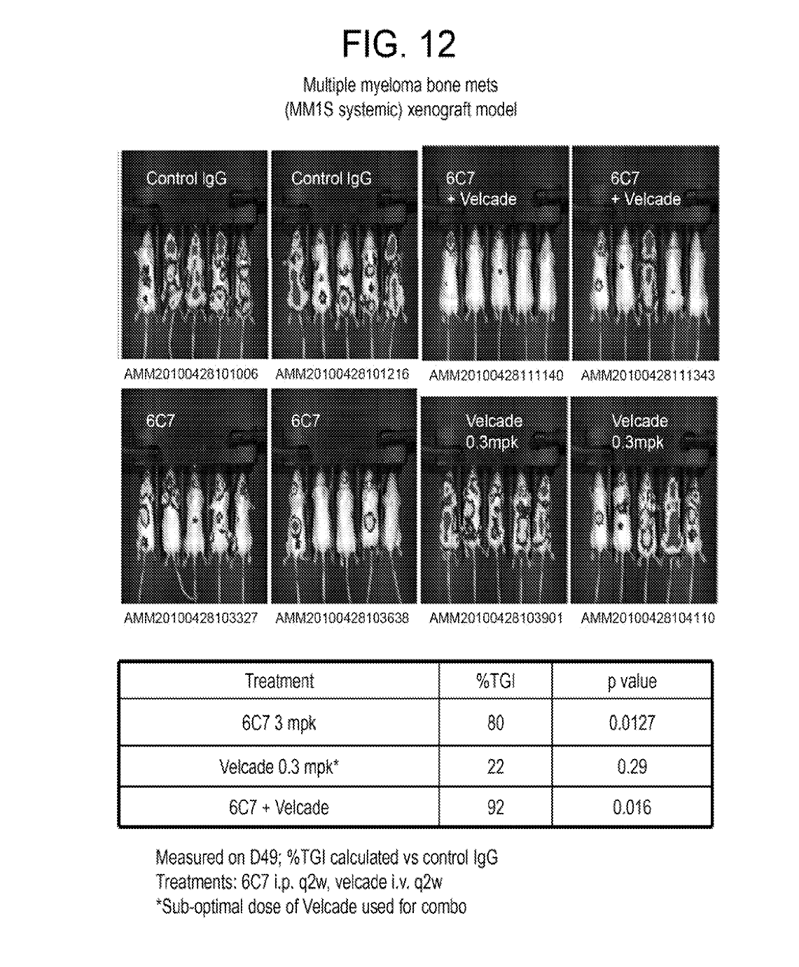

FIG. 12 depicts the results of this experiment. Decreased tumor burden is shown by a decrease in the level of bioluminescence observed. As indicated, treatment with 6C7 has significant single-agent activity with 80% TGI. When combined with a suboptimal dose of Velcade, 6C7 improves to 92% TGI.

This model is a good model for pre-established bone metastases in which efficacy in decreasing or eliminating existing metastases is evaluated.

### Example 15

### Efficacy in Burkitt's Lymphoma Xenograft Model

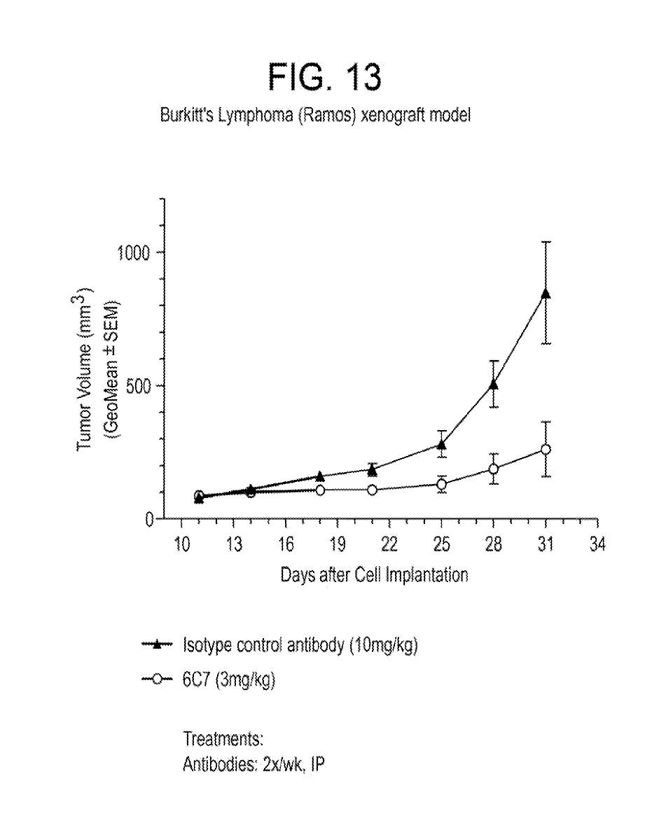

Anti-CXCR4 antibody 6C7 was also tested for its ability to inhibit human tumor growth in a xenograft model of Burkitt's lymphoma. A Ramos (human Burkitt's lymphoma) cell line was cultured at 37° C. in a CO₂ incubator in RPMI1640 media containing 10% Fetal Bovine Serum and 2% L-glutamine. 4-6 week old Nude female mice (Harlan Sprague Dawley, Indianapolis, Ind.) were injected subcutaneously. Tumors were allowed to grow to −100 mm³ and animals were randomized to control and treatment groups based on tumor size before the dosing was initiated. Tumor size was monitored by caliper measurement, and tumor volume was estimated using the formula volume=0.5×length×width². Antibody was administered intraperitoneally in a solution of sterile PBS twice per week at the indicated doses. As shown in FIG. 13, treatment of established tumors with 6C7 antibody resulted in a reduction in tumor growth.

### Example 16

### Efficacy in Ovarian Cancer Disseminated Intravenous MODEL TO LUNGS

Anti-CXCR4 antibody 6C7 was tested for its ability to inhibit human tumor growth in SCID mice using a disseminated model of ovarian cancer in the lungs. The human ovarian cancer line HeyA8Luc+Clone 4 was cultured at 37° C. in a CO₂ incubator in RPMI1640 media containing 10% fetal bovine serum and 1% L-glutamine. The results from this experiment are described in detail below.

4-6 week old SCID female mice (Harlan Sprague Dawley, Indianapolis, Ind.) were injected intravenously with HeyA8Luc+Clone 4 (1×10⁶ in PBS) in a total volume of 200 μl. Cohorts of 10 animals were randomized to control and treatment groups based on body weight before the dosing was initiated. Tumor development was monitored by weekly imaging on the IVIS® Spectrum. Mice were dosed intraperitoneally with sterile XenoLight™ D-Luciferin-K⁺ Salt at a concentration of 15 mg/mL 15 minutes prior to imaging.

6C7 was administered intraperitoneally in a solution of sterile PBS twice per week at the indicated doses. Mice were dosed either preventative or therapeutically. Treatment of disseminated tumors with 6C7 antibody blocked lung tumor growth using HeyA8 ovarian cancer cells (FIG. 14A). The preventative and therapeutic dosing were equally active. Mice treated with 6C7 using the therapeutic dosing schedule were only imaged at day 33. The scatter plot of individual lungs ex-vivo taken 33 days after initiation of treatment (FIG. 14B) and H&E staining of these lungs (dark brown=lung mets) (FIG. 14C) show that 6C7 blocks lung metastases using HeyA8 ovarian cell lines.

### Example 17

### Increase in Survival Using Chronic Lymphocytic Leukemia (CLL) Intravenous Tumor Models

Anti-CXCR4 antibody 6C7 was tested for its ability to increase survival of huCXCR4 SCID mice using a disseminated intravenous model of CLL. The human CLL cancer line JVM-2 was cultured at 37° C. in a CO₂ incubator in RPMI1640 media containing 20% fetal bovine serum and 1% L-glutamine. The results from these experiments are described in detail below.

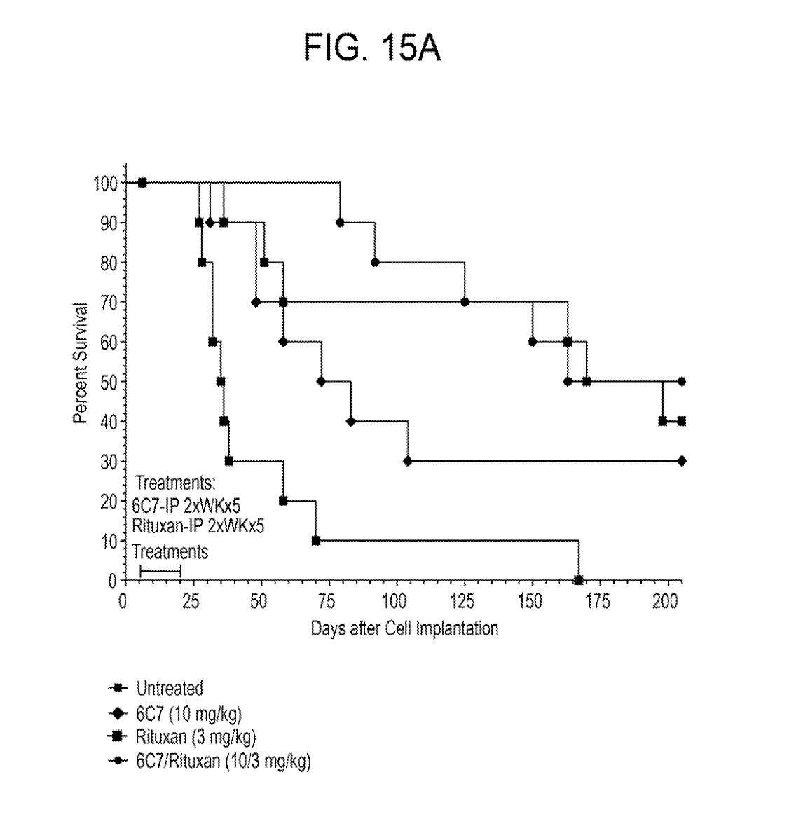

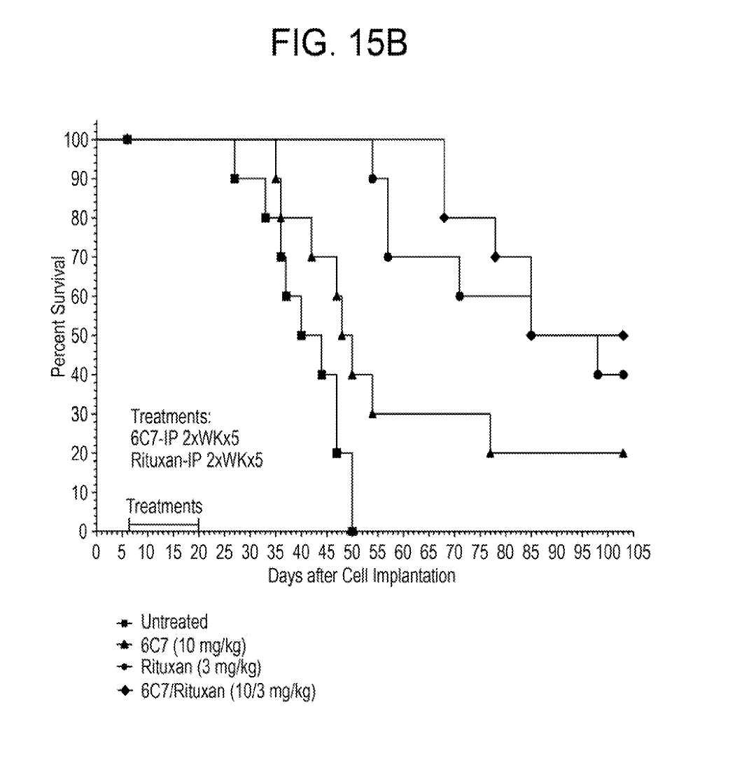

4-6 week old huCXCR4 SCID female mice (Taconic, Germantown, N.Y.) were injected intravenously with JVM-2 CLL cells (10×10⁶ in PBS) in a total volume of 200 μl. Cohorts of 10 animals were randomized to control and treatment groups based on body weight before the dosing was initiated. Antibody was administered intraperitoneally in a solution of sterile PBS twice per week at the indicated doses starting at day 6 and ending on day 21. Treatment with 6C7 antibody resulted in an increase survival over untreated animals (FIG. 15A). In a further exploration of the potential utility of CXCR4 antibody to induce tumor cell mobilization, 6C7 (10 mg/kg) antibody was administered in combination with Rituxan® (3.0 mg/kg) in the disseminated JVM-2 model using the same dosing scheme. 6C7 in combination with Rituxan® shows increase median survival of ˜135 days over untreated mice (FIG. 15A).

Anti-CXCR4 antibody 6C7 was also tested for its ability to increase survival of SCID mice using a second disseminated intravenous model of CLL. The human CLL cancer line JVM-13 was cultured at 37° C. in a CO₂ incubator in RPMI1640 media containing 20% fetal bovine serum and 1% L-glutamine. The results from these experiments are described in detail below. 4-6 week old SCID female mice (Harlan Sprague Dawley, Indianapolis, Ind.) were injected intravenously with JVM-13 CLL cells (10×10⁶ in PBS) in a total volume of 200 μl. Cohorts of 10 animals were randomized to control and treatment groups based on body weight before the dosing was initiated. Antibody was administered intraperitoneally in a solution of sterile PBS twice per week at the indicated doses starting at day 6 and ending on day 21. Treatment with 6C7 antibody resulted in a slight increase survival over untreated animals (FIG. 15B). In a further exploration of the potential utility of CXCR4 antibody to induce tumor cell mobilization, 6C7 (10 mg/kg) antibody was administered in combination with Rituxan® (3.0 mg/kg) in the disseminated JVM-13 model using the same dosing schedule. 6C7 in combination with Rituxan® shows increase median survival of −30 days over untreated mice (FIG. 15B).

### Example 18

### Epitope Mapping

Epitope mapping of CXCR4 was conducted to identify binding site for antibody 6C7.

### A. Construction and Expression of Human/Mouse Chimeric CXCR4 Variants

Swap mutants were constructed exchanging extracellular loops between human and mouse CXCR4. Mouse CXCR4 was not recognized by antibody 6C7, but shares high sequence identity with human CXCR4. Seven chimeric variants were constructed by replacing the following regions of human CXCR4 with the mouse counterparts: N-terminal peptide, 1st extracellular loop, 2nd extracellular loop, 3rd extracellular loop, N-terminal peptide and 2nd extracellular loop, N-terminal peptide and 3rd extracellular loop, 2nd and 3rd extracellular loops. The cDNAs encoding all variants were assembled and amplified by overlapping extension PCR using in-house full-length human and mouse CXCR4 plasmids as templates. The assembled cDNAs were cloned into a mammalian expression vector pcDNA3.1 (Invitrogen) and transiently expressed by transfecting the variants into CHO suspension cells using Lipofectamine LTX transfection reagent (Invitrogen) following the manufacturer's instructions.

B. Flow Cytometry Characterization of the Binding of Antibody 6C7 to these Chimeric Variants

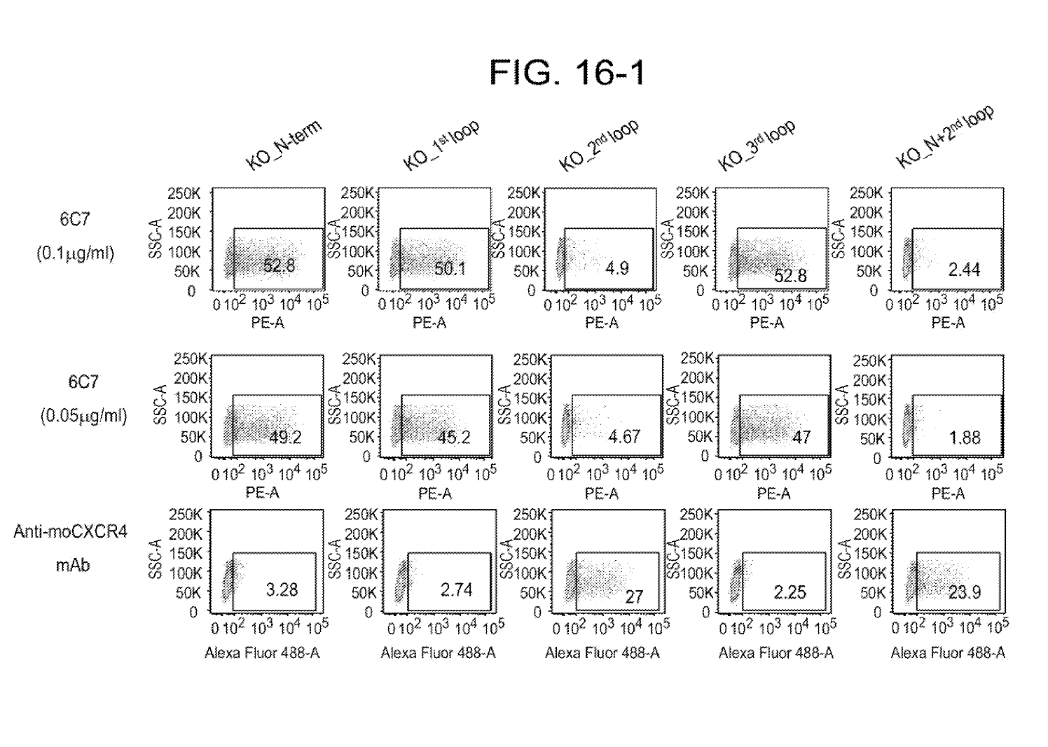

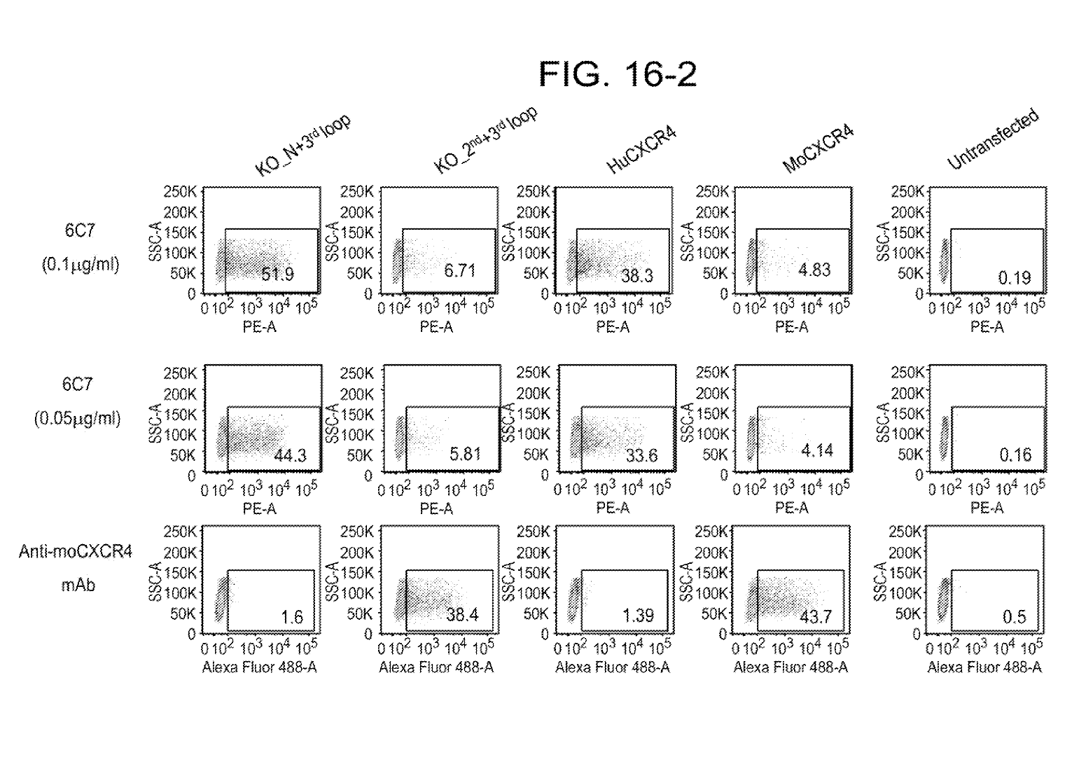

Cells transfected with human/mouse CXCR4 chimeric variant constructs were incubated with 0.5 μg/mL of antibody 6C7 for 1 hour on ice. For the detection of bound antibody 6C7, cells were washed three times with cold PBS, incubated with 1 μg/mL of anti-Human IgG antibody Alexa Fluor® 488 (Invitrogen) for 30 minutes on ice, and then analyzed using the LSRII flow cytometer (BD Biosciences). The protein expression of the variants containing a murine 2nd extracellular loop was monitored with a rat anti-mouse CXCR4 (R&D Systems) mAb followed by anti-rat IgG antibody Alexa Fluor® 488 (Invitrogen) for detection. The expression levels of all variants containing a human 2nd extracellular loop were monitored using PE conjugated anti-human CXCR4 clone 12G5 (Biolegend). Results of these experiments are shown in FIG. 16.

Domain swaps of human CXCR4 with mouse CXCR4 showed that antibody 6C7 binds the second loop of CXCR4. The second loop in human CXCR4 is shorter from mouse CXCR4 by 5 amino acids. Also, the second loop has 7 individual residue differences. The first of these single amino acid differences results in loss of an N-glycosylation consensus sequence that is present in human but not in mouse.

All publications, patents and patent applications mentioned in this specification are herein incorporated by reference into the specification to the same extent as if each individual publication, patent or patent application was specifically and individually indicated to be incorporated herein by reference.

SEQ ID NO: 1

| | |
|---|---|
| caggtgcagc tggtggagtc tgggggaggc gtggtccagc | 60 |
| ctgggaggtc cctgagactc | |
| tcctgtgcag cctctggatt caccttcagt agctatggca | 120 |
| tgcactgggt ccgccaggct | |
| ccaggcaagg ggctggagtg ggtggcagtt atatcatatg | 180 |
| atggaagtaa taaatactat | |
| gcagactccg tgaagggccg attcaccatc tccagagaca | 240 |
| attccaagaa cacgctgtat | |
| ctgcaaatga acagcctgag agctgaggac acggctgtgt | 300 |
| attactgtac gaggggaggt | |
| ttagcagctc gccggaatta ctactacagc tacggtatgg | 360 |
| acgtctgggg ccaagggacc | |
| acggtcaccg tctcctca | 378 |

SEQ ID NO: 2

Gln Val Gln Leu Val Glu Ser Gly Gly Gly Val
Val Gln Pro Gly Arg Ser Leu Arg Leu Ser Cys
Ala Ala Ser Gly Phe Thr Phe Ser Ser Tyr Gly
Met His Trp Val Arg Gln Ala Pro Gly Lys Gly
Leu Glu Trp Val Ala Val Ile Ser Tyr Asp Gly
Ser Asn Lys Tyr Tyr Ala Asp Ser Val Lys Gly
Arg Phe Thr Ile Ser Arg Asp Asn Ser Lys Asn
Thr Leu Tyr Leu Gln Met Asn Ser Leu Arg Ala
Glu Asp Thr Ala Val Tyr Tyr Cys Thr Arg Gly
Gly Leu Ala Ala Arg Arg Asn Tyr Tyr Tyr Ser
Tyr Gly Met Asp Val Trp Gly Gln Gly Thr Thr
Val Thr Val Ser Ser

SEQ ID NO: 3

| | |
|---|---|
| gacatccaga tgacccagtc tccatcctcc ctgtctgcat | 60 |
| ctgtaggaga cagagtcacc | |
| atcacttgcc gggcaagtca ggacattaga aatgatttag | 120 |
| gctggtatca gcagaaacca | |
| gggaaagccc ctaagcgcct gatctatgct gcatccagtt | 180 |
| tgcaaagtgg ggtcccatca | |
| aggttcagcg gcagtggatc tgggacacaa ttcactctca | 240 |
| caatcagcag cctgcagcct | |
| gaagattttg caacttatta ctgtctacag cataatagtt | 300 |
| accctctcac tttcggcgga | |
| gggaccaagg tgcagatcaa a | 321 |

SEQ ID NO: 4

Asp Ile Gln Met Thr Gln Ser Pro Ser Ser Leu
Ser Ala Ser Val Gly Asp Arg Val Thr Ile Thr
Cys Arg Ala Ser Gln Asp Ile Arg Asn Asp Leu
Gly Trp Tyr Gln Gln Lys Pro Gly Lys Ala Pro
Lys Arg Leu Ile Tyr Ala Ala Ser Ser Leu Gln
Ser Gly Val Pro Ser Arg Phe Ser Gly Ser Gly
Ser Gly Thr Gln Phe Thr Leu Thr Ile Ser Ser
Leu Gln Pro Glu Asp Phe Ala Thr Tyr Tyr Cys
Leu Gln His Asn Ser Tyr Pro Leu Thr Phe Gly
Gly Gly Thr Lys Val Gln Ile Lys

SEQ ID NO: 5

| | |
|---|---|
| caggtgcagc tggtggagtc tgggggaggc gtggtccagc | 60 |
| ctgggaggtc cctgagactc | |
| tcctgtgcag cgtctggatt caccttcagt aactatgtca | 120 |
| tgcactgggt ccgccaggct | |
| ccaggcaagg ggctggagtg ggtggcagtt atatggtatg | 180 |
| atggaagtaa taaatactat | |
| gcagactccg tgaagggccg attcaccatc tccagagaca | 240 |
| attccaagaa cacgctgtct | |
| ctgcaaatga acagcctgag agccgaggac acggctgtat | 300 |
| attactgtga gagaggggaa | |
| gggtactatg gctcggggag tcgttataga ggctactact | 360 |
| acggtatgga cgtctggggc | |
| caagggacca cggtcaccgt ctcctca | 387 |

SEQ ID NO: 6

Gln Val Gln Leu Val Glu Ser Gly Gly Gly Val
Val Gln Pro Gly Arg Ser Leu Arg Leu Ser Cys
Ala Ala Ser Gly Phe Thr Phe Ser Asn Tyr Val
Met His Trp Val Arg Gln Ala Pro Gly Lys Gly
Leu Glu Trp Val Ala Val Ile Trp Tyr Asp Gly
Ser Asn Lys Tyr Tyr Ala Asp Ser Val Lys Gly
Arg Phe Thr Ile Ser Arg Asp Asn Ser Lys Asn
Thr Leu Ser Leu Gln Met Asn Ser Leu Arg Ala
Glu Asp Thr Ala Val Tyr Tyr Cys Glu Arg Gly
Glu Gly Tyr Tyr Gly Ser Gly Ser Arg Tyr Arg
Gly Tyr Tyr Tyr Gly Met Asp Val Trp Gly Gln
Gly Thr Thr Val Thr Val Ser Ser

SEQ ID NO: 7

| | |
|---|---|
| gacatccaga tgacccagtc tccatcctcc ctgtctgcat | 60 |
| ctgtaggaga cagagtcacc | |
| atcacttgcc gggcaagtca gggcattaga actgatttag | 120 |
| gctggtatca gcagaaacca | |
| gggaaagccc ctaagcgcct gatctatgct gcatccagtt | 180 |
| tgcaaagtgg ggtcccatca | |
| aggttcagcg gcagtggatc tgggacagaa ttcactctca | 240 |
| caatcagcag cctgcagccc | |
| gaagattttg caacttatta ctgtctacag cataatagtt | 300 |
| accctcggac attcggccaa | |
| gggaccaagg tggaaatcaa a | 321 |

SEQ ID NO: 8

Asp Ile Gln Met Thr Gln Ser Pro Ser Ser Leu
Ser Ala Ser Val Gly Asp Arg Val Thr Ile Thr
Cys Arg Ala Ser Gln Gly Ile Arg Thr Asp Leu
Gly Trp Tyr Gln Gln Lys Pro Gly Lys Ala Pro
Lys Arg Leu Ile Tyr Ala Ala Ser Ser Leu Gln
Ser Gly Val Pro Ser Arg Phe Ser Gly Ser Gly
Ser Gly Thr Glu Phe Thr Leu Thr Ile Ser Ser
Leu Gln Pro Glu Asp Phe Ala Thr Tyr Tyr Cys
Leu Gln His Asn Ser Tyr Pro Arg Thr Phe Gly
Gln Gly Thr Lys Val Glu Ile Lys

SEQ ID NO: 9

| | |
|---|---|
| caggtgcagc tggtggagtc tgggggaggc gtggtccagc | 60 |
| ctgggaggtc cctgagactc | |
| tcctgtgcag cctctggatt caccttcagt agctatggca | 120 |
| tgcactgggt ccgccaggct | |
| ccaggcaagg ggctggagtg ggtggcagtt atatcatatg | 180 |
| atggaagtaa taaatactat | |
| gcagactccg tgaagggccg attcaccatc tccagagaca | 240 |
| attccaagaa cacgctgtat | |
| ctgcaaatga acagcctgag agctgaggac acggctgtgt | 300 |
| attattgtac gaggggaggt | |
| ttagcagctc gccggaatta ctactacagc tacggtacgg | 360 |
| acgtctgggg ccaagggacc | |
| acggtcaccg tctcctca | 378 |

SEQ ID NO: 10

Gln Val Gln Leu Val Glu Ser Gly Gly Gly Val
Val Gln Pro Gly Arg Ser Leu Arg Leu Ser Cys
Ala Ala Ser Gly Phe Thr Phe Ser Ser Tyr Gly
Met His Trp Val Arg Gln Ala Pro Gly Lys Gly
Leu Glu Trp Val Ala Val Ile Ser Tyr Asp Gly
Ser Asn Lys Tyr Tyr Ala Asp Ser Val Lys Gly
Arg Phe Thr Ile Ser Arg Asp Asn Ser Lys Asn
Thr Leu Tyr Leu Gln Met Asn Ser Leu Arg Ala
Glu Asp Thr Ala Val Tyr Tyr Cys Thr Arg Gly
Gly Leu Ala Ala Arg Arg Asn Tyr Tyr Tyr Ser
Tyr Gly Thr Asp Val Trp Gly Gln Gly Thr Thr
Val Thr Val Ser Ser

SEQ ID NO: 11

| | |
|---|---|
| gacatccaga tgacccagtc tccatcctcc ctgtctgcat | 60 |
| ctgtaggaga cagagtcacc | |
| atcacttgcc gggcaagtca ggacattaga aatgatttag | 120 |
| gctggtatca gcagaaaccg | |
| gggaaagccc ctacgcgcct gatctatgct gcatccagtt | 180 |
| tgcaaagtgg ggtcccatca | |
| cggttcagcg gcagtggatc tgggacacaa ttcactctca | 240 |
| caatcagcag cctgcagcct | |
| gaagattttg caacttatta ctgtctacag cataatagtt | 300 |
| accctctcac tttcggcgga | |
| gggaccaagg tgcagatcaa a | 321 |

SEQ ID NO: 12

Asp Ile Gln Met Thr Gln Ser Pro Ser Ser Leu
Ser Ala Ser Val Gly Asp Arg Val Thr Ile Thr
Cys Arg Ala Ser Gln Asp Ile Arg Asn Asp Leu
Gly Trp Tyr Gln Gln Lys Pro Gly Lys Ala Pro
Thr Arg Leu Ile Tyr Ala Ala Ser Ser Leu Gln
Ser Gly Val Pro Ser Arg Phe Ser Gly Ser Gly
Ser Gly Thr Gln Phe Thr Leu Thr Ile Ser Ser
Leu Gln Pro Glu Asp Phe Ala Thr Tyr Tyr Cys
Leu Gln His Asn Ser Tyr Pro Leu Thr Phe Gly
Gly Gly Thr Lys Val Gln Ile Lys

SEQ ID NO: 13

| | |
|---|---|
| caggtgcagc tggtggagtc tgggggaggc gtggtccagc | 60 |
| ctgggaggtc cctgagactc | |
| tcctgtgcag cctctggatt caccttcagt agctatggct | 120 |
| tgcactgggt ccgccagtct | |
| ccaggcaagg ggctggagtg ggtggcagtt atatcatatg | 180 |
| atggaagtaa aaaatactat | |
| gcagactccg tgaagggccg attcagcatc tccagagaca | 240 |
| attccaagaa cacgctgtat | |
| ctgcaaatga acagcctgag agctgaggac acggctgtgt | 300 |
| attactgtgc gagagatcgc | |
| ccttcacgat attcctcctg tatggacgtc tggggccaag | 360 |
| ggaccacggt caccgtctcc | |
| tca | 363 |

SEQ ID NO: 14

Gln Val Gln Leu Val Glu Ser Gly Gly Gly Val
Val Gln Pro Gly Arg Ser Leu Arg Leu Ser Cys
Ala Ala Ser Gly Phe Thr Phe Ser Ser Tyr Gly
Leu His Trp Val Arg Gln Ser Pro Gly Lys Gly
Leu Glu Trp Val Ala Val Ile Ser Tyr Asp Gly
Ser Lys Lys Tyr Tyr Ala Asp Ser Val Lys Gly
Arg Phe Ser Ile Ser Arg Asp Asn Ser Lys Asn
Thr Leu Tyr Leu Gln Met Asn Ser Leu Arg Ala
Glu Asp Thr Ala Val Tyr Tyr Cys Ala Arg Asp
Arg Pro Ser Arg Tyr Ser Ser Cys Met Asp Val
Trp Gly Gln Gly Thr Thr Val Thr Val Ser Ser

SEQ ID NO: 15

| | |
|---|---|
| cagtctgccc tgactcagcc tgcctccgtg tctgggtctc | 60 |
| ctggacagtc gatcaccatc | |
| tcctgcactg gaaccagcag tgatgttggg agtaataact | 120 |
| ttgtctcctg gtaccaacag | |
| cacccaggca aagcccccaa actcatgatt tatgaggtca | 180 |
| gtaagcggcc ctcaggggtt | |
| tctaatcgct tctctggctc caagtctggc aacacggcct | 240 |
| ccctgacaat ctctgggctc | |
| caggctgagg acgaggctga ttattactgc tgctcatatg | 300 |
| caggtagtaa cactttggtg | |
| ttcggcggag ggaccaaact gaccgtccta | 330 |

SEQ ID NO: 16

Gln Ser Ala Leu Thr Gln Pro Ala Ser Val Ser
Gly Ser Pro Gly Gln Ser Ile Thr Ile Ser Cys
Thr Gly Thr Ser Ser Asp Val Gly Ser Asn Asn
Phe Val Ser Trp Tyr Gln Gln His Pro Gly Lys
Ala Pro Lys Leu Met Ile Tyr Glu Val Ser Lys
Arg Pro Ser Gly Val Ser Asn Arg Phe Ser Gly
Ser Lys Ser Gly Asn Thr Ala Ser Leu Thr Ile
Ser Gly Leu Gln Ala Glu Asp Glu Ala Asp Tyr
Tyr Cys Cys Ser Tyr Ala Gly Ser Asn Thr Leu
Val Phe Gly Gly Gly Thr Lys Leu Thr Val Leu

SEQ ID NO: 17

| | |
|---|---|
| gaggtacagc tgttggagtc tgggggaggc ttggtacagc | 60 |
| ctggggggtc cctgagactc | |
| tcctgtgcag cctctggatt cacctttagc agctttgcca | 120 |
| tgaattgggt ccgccaggct | |
| ccagggaagg ggctggagtg ggtctcagct attagtggta | 180 |
| gtggtggtaa tatatattac | |
| gcagactccg tgaggggccg gttcaccatc tccagagaca | 240 |
| attccaagaa cacgctgtat | |
| ctgcaaatga acagcctgag agccgaggac acggccgtat | 300 |
| attactgtgc gaaagtcgac | |
| aggaacttag gatactatca cggtatggac gtctggggcc | 360 |
| aagggaccac ggtcaccgtc | |
| tcctca | 366 |

SEQ ID NO: 18

Glu Val Gln Leu Leu Glu Ser Gly Gly Gly Leu
Val Gln Pro Gly Gly Ser Leu Arg Leu Ser Cys
Ala Ala Ser Gly Phe Thr Phe Ser Ser Phe Ala
Met Asn Trp Val Arg Gln Ala Pro Gly Lys Gly
Leu Glu Trp Val Ser Ala Ile Ser Gly Ser Gly
Gly Asn Ile Tyr Tyr Ala Asp Ser Val Arg Gly
Arg Phe Thr Ile Ser Arg Asp Asn Ser Lys Asn
Thr Leu Tyr Leu Gln Met Asn Ser Leu Arg Ala
Glu Asp Thr Ala Val Tyr Tyr Cys Ala Lys Val
Asp Arg Asn Leu Gly Tyr Tyr His Gly Met Asp
Val Trp Gly Gln Gly Thr Thr Val Thr Val Ser
Ser

SEQ ID NO: 19

| | |
|---|---|
| gatattgtga tgactcagtc tccactctcc ctgcccgtca | 60 |
| cccctggaga gccggcctcc | |
| atctcctgca ggtctagtca gagcctcctg catagtaatg | 120 |
| gatacaacta tttggattgg | |
| tacctgcaga agccagggca gtctccacaa ctcctgatct | 180 |
| atttgggttc taatcgggcc | |
| tccggggtcc ctgacaggtt cagtggcagt ggatctggct | 240 |
| cagattttac actgaaaatc | |
| agcagagtgg aggctgagga tgttggagtt tattactgca | 300 |
| tgcaagctct acaattcact | |
| ttcggccctg ggaccaaagt ggatatcaaa | 330 |

SEQ ID NO: 20

Asp Ile Val Met Thr Gln Ser Pro Leu Ser Leu
Pro Val Thr Pro Gly Glu Pro Ala Ser Ile Ser
Cys Arg Ser Ser Gln Ser Leu Leu His Ser Asn
Gly Tyr Asn Tyr Leu Asp Trp Tyr Leu Gln Lys
Pro Gly Gln Ser Pro Gln Leu Leu Ile Tyr Leu
Gly Ser Asn Arg Ala Ser Gly Val Pro Asp Arg
Phe Ser Gly Ser Gly Ser Gly Ser Asp Phe Thr
Leu Lys Ile Ser Arg Val Glu Ala Glu Asp Val
Gly Val Tyr Tyr Cys Met Gln Ala Leu Gln Phe
Thr Phe Gly Pro Gly Thr Lys Val Asp Ile Lys

SEQ ID NO: 21

| | |
|---|---|
| caggtgcagc tggtggagtc tgggggaggc gtggtccagc | 60 |
| ctgggaggtc cctgagactc | |
| tcctgtgcag cgtcaggatt caccttcagt agctatggca | 120 |
| tgcactgggt ccgccaggct | |
| ccaggcaagg gactggagtg ggtggcagtt atatggtatg | 180 |
| atggaactta taaatactat | |
| gcagactccg tgaggggccg attcaccatc tccagagaca | 240 |
| attccaagaa cacgctgtat | |
| ctgcaaatga acagcctgag agccgaggac acggctgtat | 300 |
| attactgtgc gagggggccc | |
| ctattacgat attttgactg gttatccgac tactggggcc | 360 |
| agggaaccct ggtcaccgtc | |
| tcctca | 366 |

SEQ ID NO: 22

Gln Val Gln Leu Val Glu Ser Gly Gly Gly Val
Val Gln Pro Gly Arg Ser Leu Arg Leu Ser Cys
Ala Ala Ser Gly Phe Thr Phe Ser Ser Tyr Gly
Met His Trp Val Arg Gln Ala Pro Gly Lys Gly
Leu Glu Trp Val Ala Val Ile Trp Tyr Asp Gly
Thr Tyr Lys Tyr Tyr Ala Asp Ser Val Arg Gly
Arg Phe Thr Ile Ser Arg Asp Asn Ser Lys Asn
Thr Leu Tyr Leu Gln Met Asn Ser Leu Arg Ala
Glu Asp Thr Ala Val Tyr Tyr Cys Ala Arg Gly
Pro Leu Leu Arg Tyr Phe Asp Trp Leu Ser Asp
Tyr Trp Gly Gln Gly Thr Leu Val Thr Val Ser
Ser

SEQ ID NO: 23

| | |
|---|---|
| gacatccaga tgacccagtc tccatcctcc ctgtctgcat | 60 |
| ctgtaggaga cagagtcacc | |
| atcacttgcc gggcaagtca gggcattaga aatgatttag | 120 |
| gctggtatca gcagaaacca | |
| gggaaagccc ctaagcgcct gatctatgct gcaaccagtt | 180 |
| tgcaaagtgg ggtcccatca | |
| cggttcagcg gcagtggatc tgggacagaa ttcactctca | 240 |
| caatcagcag cctgcagcct | |
| gaagattttg caacctatta ctgtctacag cataataatt | 300 |
| atccgcgcac tttcggcgga | |
| gggaccaagg tggagatcaa a | 321 |

SEQ ID NO: 24

Asp Ile Gln Met Thr Gln Ser Pro Ser Ser Leu
Ser Ala Ser Val Gly Asp Arg Val Thr Ile Thr
Cys Arg Ala Ser Gln Gly Ile Arg Asn Asp Leu
Gly Trp Tyr Gln Gln Lys Pro Gly Lys Ala Pro
Lys Arg Leu Ile Tyr Ala Ala Thr Ser Leu Gln
Ser Gly Val Pro Ser Arg Phe Ser Gly Ser Gly
Ser Gly Thr Glu Phe Thr Leu Thr Ile Ser Ser
Leu Gln Pro Glu Asp Phe Ala Thr Tyr Tyr Cys
Leu Gln His Asn Asn Tyr Pro Arg Thr Phe Gly
Gly Gly Thr Lys Val Glu Ile Lys

1-26. (canceled)

27. An isolated antibody or antigen binding fragment comprising an amino acid sequence comprising:
a heavy chain variable domain sequence comprising at least one, at least two, or at least three of the heavy chain CDRs encoded by the polynucleotide in plasmid designated Mab6C7VH which was deposited at the American Type Culture Collection (ATCC) under number PTA-9630 on Nov. 18, 2008;
a light chain variable domain sequence comprising at least one, at least two, or at least three of the light chain CDRs encoded by the polynucleotide in plasmid designated Mab6C7VH which was deposited at the American Type Culture Collection (ATCC) under number PTA-9631 on Nov. 18, 2008; or
a heavy chain variable domain sequence comprising at least one, at least two, or at least three of the heavy chain CDRs encoded by the polynucleotide in plasmid designated Mab6C7VH which was deposited at the American Type Culture Collection (ATCC) under number PTA-9630 on Nov. 18, 2008 and a light chain variable domain sequence comprising at least one, at least two, or at least three of the light chain CDRs of the antibody encoded by the polynucleotide in plasmid designated Mab6C7VL which was deposited at the American Type Culture Collection (ATCC) under number PTA-9631 on Nov. 18, 2008.

28. A composition comprising the antibody or antigen binding fragment of claim 27.

29. A pharmaceutical composition comprising the antibody or antigen binding fragment of claim 27.

30. (canceled)

31. A method of treating a malignant tumour in an animal, comprising: selecting an animal in need of treatment for a malignant tumour; and administering to the animal a therapeutically effective dose of the antibody or antigen binding fragment of claim 27.

32-35. (canceled)

36. The method of claim 31, further comprising administration of a platinum drug or a taxane as part of a therapeutic regimen

37. A CXCR4 antibody or antigen binding fragment thereof comprising:
a variable heavy (VH) domain complementarity determining region (CDR) 1 comprising amino acids 31-35 of SEQ ID NO. 6; a VH CDR2 comprising amino acids 50-66 of SEQ ID NO. 6; a VH CDR3 comprising amino acids 99-118 of SEQ ID NO. 6; a variable light (VL) CDR1 comprising amino acids 24-34 of SEQ ID NO. 8; a VL CDR2 comprising amino acids 50-56 of SEQ ID NO. 8; and a VL CDR3 comprising amino acids 89-97 of SEQ ID NO. 8.

38. The CXCR4 antibody or antigen binding fragment thereof according to claim 37, comprising a VH domain comprising the amino acid sequence as shown in SEQ ID NO. 6 and a VL domain comprising the amino acid sequence as shown in SEQ ID NO. 8.

39. The CXCR4 antibody or antigen binding fragment thereof according to claim 37, wherein the antibody or antigen binding fragment exhibits one or more of the following properties selected from the group consisting of:
binds human CXCR4 with a K_{D} of less than 2.5 nanomolar (nM) when measured by FACS binding kinexa analysis;
binds to the second extracellular loop of human CXCR4;
cross-reacts with cynomolgus monkey CXCR4 with a a K_{D} of less than 1 nM when measured by FACS binding kinexa analysis;
does not bind significantly to CXCR3 or CCR4;
inhibits SDF-1 binding to CXCR4;
inhibits SDF-1 induced pMAPK phosphorylation;
inhibits SDF-1 induced Jurkat chemotaxis with an 1050 of less than 0.5 nM;
inhibits SDF-1 induced HUVEC migration at an 1050 concentration of below 10 nM;
inhibits AKT phosphorylation in HSC-F cells or MDA-MB-231 cells;
induces apoptosis in Ramos cells; and
causes no more than a 60% reduction of B-cell counts when added to a peripheral blood leukocyte cell preparation at a concentration of 10 ug/ml over a period of 16-18 hours.

40. The CXCR4 antibody or antigen binding fragment thereof according to claim 37, wherein the antibody or antigen binding fragment binds CXCR4 with a Kd of less than 1 nM.

41. The CXCR4 antibody or antigen binding fragment thereof according to claim 37, wherein the antibody is a human or humanized antibody.

42. The CXCR4 antibody or antigen binding fragment thereof according to claim 37, wherein the antibody is conjugated to a label or a therapeutic agent.

43. The CXCR4 antibody or antigen binding fragment thereof according to claim 37, wherein the antibody or antigen binding fragment thereof is a Fab, Fab′, F(ab′)2, Fv or dAb.

44. A nucleic acid molecule encoding the antibody or antigen binding fragment of claim 37.

45. A nucleic acid molecule encoding the antibody or antigen binding fragment of claim 38.

46. A pharmaceutical composition comprising the CXCR4 antibody or antigen binding fragment thereof according to claim 37.

47. A pharmaceutical composition comprising the CXCR4 antibody or antigen binding fragment thereof according to claim 38.

48. A method of treating a malignant tumor in an animal, comprising: selecting an animal in need of treatment for a malignant tumor; and administering to the animal a therapeutically effective dose of the antibody or antigen binding fragment thereof according to claim 37.

49. A method of treating a malignant tumor in an animal, comprising: selecting an animal in need of treatment for a malignant tumor; and administering to the animal a therapeutically effective dose of the antibody or antigen binding fragment thereof according to claim 38.

50. The method of claim 48, wherein said antibody or antigen binding fragment is administered as part of a therapeutic regimen in combination with standard of care.

51. The method of claim 49, wherein said antibody or antigen binding fragment is administered as part of a therapeutic regimen in combination with standard of care.

52. The method of claim 50, further comprising administration of a platinum drug or a taxane as part of the therapeutic regimen.

53. The method of claim 51, further comprising administration of a platinum drug or a taxane as part of the therapeutic regimen.


SEQUENCE LISTING (60 sequences)

SEQ ID NO: 1 - DNA, 378 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtacgaggggaggtttagcagctcgccggaattactactacagctacggtatggacgtctggggccaagggaccacggtcaccgtctcctca

SEQ ID NO: 2 - protein, 126 aa, Artificial Sequence
  Description of Artificial Sequence Synthetic polypeptide
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCTRGGLAARRNYYYSYGMDVWGQGTTVTVSS

SEQ ID NO: 3 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcaggacattagaaatgatttaggctggtatcagcagaaaccagggaaagcccctaagcgcctgatctatgctgcatccagtttgcaaagtggggtcccatcaaggttcagcggcagtggatctgggacacaattcactctcacaatcagcagcctgcagcctgaagattttgcaacttattactgtctacagcataatagttaccctctcactttcggcggagggaccaaggtgcagatcaaa

SEQ ID NO: 4 - protein, 107 aa, Artificial Sequence
  Description of Artificial Sequence Synthetic polypeptide
DIQMTQSPSSLSASVGDRVTITCRASQDIRNDLGWYQQKPGKAPKRLIYAASSLQSGVPSRFSGSGSGTQFTLTISSLQPEDFATYYCLQHNSYPLTFGGGTKVQIK

SEQ ID NO: 5 - DNA, 387 bp, Artificial Sequence
  Description of Artificial Sequence Synthetic polynucleotide
caggtgcagctggtggagtctgggggaggcgtggtccagcctgggaggtccctgagactctcctgtgcagcgtctggattcaccttcagtaactatgtcatgcactgggtccgccaggctccaggcaaggggctggagtgggtggcagttatatggtatgatggaagtaataaatactatgcagactccgtgaagggccgattcaccatctccagagacaattccaagaacacgctgtctctgcaaatgaacagcctgagagccgaggacacggctgtatattactgtgagagaggggaagggtactatggctcggggagtcgttatagaggctactactacggtatggacgtctggggccaagggaccacggtcaccgtctcctca

SEQ ID NO: 6 - protein, 129 aa, Artificial Sequence
  Description of Artificial Sequence Synthetic polypeptide
QVQLVESGGGVVQPGRSLRLSCAASGFTFSNYVMHWVRQAPGKGLEWVAVIWYDGSNKYYADSVKGRFTISRDNSKNTLSLQMNSLRAEDTAVYYCERGEGYYGSGSRYRGYYYGMDVWGQGTTVTVSS

SEQ ID NO: 7 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagggcattagaactgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcccgaagattttgcaacttattactgtctacagcataatagttaccctcggacattcggccaagggaccaaggtggaaatcaaa

SEQ ID NO: 8 - protein, 107 aa, Artificial Sequence
  Description of Artificial Sequence Synthetic polypeptide
DIQMTQSPSSLSASVGDRVTITCRASQGIRTDLGWYQQKPGKAPKRLIYAASSLQSGVPSRFSGSGSGTEFTLTISSLQPEDFATYYCLQHNSYPRTFGQGTKVEIK

SEQ ID NO: 9 - DNA, 378 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattattgtacgaggggaggtttagcagctcgccggaattactactacagctacggtacggacgtctggggccaagggaccacggtcaccgtctcctca

SEQ ID NO: 10 - protein, 126 aa, Artificial Sequence
  Description of Artificial Sequence Synthetic polypeptide
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCTRGGLAARRNYYYSYGTDVWGQGTTVTVSS

SEQ ID NO: 11 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcaggacattagaaatgatttaggctggtatcagcagaaaccggggaaagcccctacgcgcctgatctatgctgcatccagtttgcaaagtggggtcccatcacggttcagcggcagtggatctgggacacaattcactctcacaatcagcagcctgcagcctgaagattttgcaacttattactgtctacagcataatagttaccctctcactttcggcggagggaccaaggtgcagatcaaa

SEQ ID NO: 12 - protein, 107 aa, Artificial Sequence
  Description of Artificial Sequence Synthetic polypeptide
DIQMTQSPSSLSASVGDRVTITCRASQDIRNDLGWYQQKPGKAPTRLIYAASSLQSGVPSRFSGSGSGTQFTLTISSLQPEDFATYYCLQHNSYPLTFGGGTKVQIK

SEQ ID NO: 13 - DNA, 363 bp, Artificial Sequence
  Description of Artificial Sequence Synthetic polynucleotide
caggtgcagctggtggagtctgggggaggcgtggtccagcctgggaggtccctgagactctcctgtgcagcctctggattcaccttcagtagctatggcttgcactgggtccgccagtctccaggcaaggggctggagtgggtggcagttatatcatatgatggaagtaaaaaatactatgcagactccgtgaagggccgattcagcatctccagagacaattccaagaacacgctgtatctgcaaatgaacagcctgagagctgaggacacggctgtgtattactgtgcgagagatcgcccttcacgatattcctcctgtatggacgtctggggccaagggaccacggtcaccgtctcctca

SEQ ID NO: 14 - protein, 121 aa, Artificial Sequence
  Description of Artificial Sequence Synthetic polypeptide
QVQLVESGGGVVQPGRSLRLSCAASGFTFSSYGLHWVRQSPGKGLEWVAVISYDGSKKYYADSVKGRFSISRDNSKNTLYLQMNSLRAEDTAVYYCARDRPSRYSSCMDVWGQGTTVTVSS

SEQ ID NO: 15 - DNA, 330 bp, Artificial Sequence
  Description of Artificial Sequence Synthetic polynucleotide
cagtctgccctgactcagcctgcctccgtgtctgggtctcctggacagtcgatcaccatctcctgcactggaaccagcagtgatgttgggagtaataactttgtctcctggtaccaacagcacccaggcaaagcccccaaactcatgatttatgaggtcagtaagcggccctcaggggtttctaatcgcttctctggctccaagtctggcaacacggcctccctgacaatctctgggctccaggctgaggacgaggctgattattactgctgctcatatgcaggtagtaacactttggtgttcggcggagggaccaaactgaccgtccta

SEQ ID NO: 16 - protein, 110 aa, Artificial Sequence
  Description of Artificial Sequence Synthetic polypeptide
QSALTQPASVSGSPGQSITISCTGTSSDVGSNNFVSWYQQHPGKAPKLMIYEVSKRPSGVSNRFSGSKSGNTASLTISGLQAEDEADYYCCSYAGSNTLVFGGGTKLTVL

SEQ ID NO: 17 - DNA, 366 bp, Artificial Sequence
  Description of Artificial Sequence Synthetic polynucleotide
gaggtacagctgttggagtctgggggaggcttggtacagcctggggggtccctgagactctcctgtgcagcctctggattcacctttagcagctttgccatgaattgggtccgccaggctccagggaaggggctggagtgggtctcagctattagtggtagtggtggtaatatatattacgcagactccgtgaggggccggttcaccatctccagagacaattccaagaacacgctgtatctgcaaatgaacagcctgagagccgaggacacggccgtatattactgtgcgaaagtcgacaggaacttaggatactatcacggtatggacgtctggggccaagggaccacggtcaccgtctcctca

SEQ ID NO: 18 - protein, 122 aa, Artificial Sequence
  Description of Artificial Sequence Synthetic polypeptide
EVQLLESGGGLVQPGGSLRLSCAASGFTFSSFAMNWVRQAPGKGLEWVSAISGSGGNIYYADSVRGRFTISRDNSKNTLYLQMNSLRAEDTAVYYCAKVDRNLGYYHGMDVWGQGTTVTVSS

SEQ ID NO: 19 - DNA, 330 bp, Artificial Sequence
  Description of Artificial Sequence Synthetic polynucleotide
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaactcctgatctatttgggttctaatcgggcctccggggtccctgacaggttcagtggcagtggatctggctcagattttacactgaaaatcagcagagtggaggctgaggatgttggagtttattactgcatgcaagctctacaattcactttcggccctgggaccaaagtggatatcaaa

SEQ ID NO: 20 - protein, 110 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGSDFTLKISRVEAEDVGVYYCMQALQFTFGPGTKVDIK

SEQ ID NO: 21 - DNA, 366 bp, Artificial Sequence
  Description of Artificial Sequence Synthetic polynucleotide
caggtgcagctggtggagtctgggggaggcgtggtccagcctgggaggtccctgagactctcctgtgcagcgtcaggattcaccttcagtagctatggcatgcactgggtccgccaggctccaggcaagggactggagtgggtggcagttatatggtatgatggaacttataaatactatgcagactccgtgaggggccgattcaccatctccagagacaattccaagaacacgctgtatctgcaaatgaacagcctgagagccgaggacacggctgtatattactgtgcgagggggcccctattacgatattttgactggttatccgactactggggccagggaaccctggtcaccgtctcctca

SEQ ID NO: 22 - protein, 122 aa, Artificial Sequence
  Description of Artificial Sequence Synthetic polypeptide
QVQLVESGGGVVQPGRSLRLSCAASGFTFSSYGMHWVRQAPGKGLEWVAVIWYDGTYKYYADSVRGRFTISRDNSKNTLYLQMNSLRAEDTAVYYCARGPLLRYFDWLSDYWGQGTLVTVSS

SEQ ID NO: 23 - DNA, 321 bp, Artificial Sequence
  Description of Artificial Sequence Synthetic polynucleotide
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcaaccagtttgcaaagtggggtcccatcacggttcagcggcagtggatctgggacagaattcactctcacaatcagcagcctgcagcctgaagattttgcaacctattactgtctacagcataataattatccgcgcactttcggcggagggaccaaggtggagatcaaa

SEQ ID NO: 24 - protein, 107 aa, Artificial Sequence
  Description of Artificial Sequence Synthetic polypeptide
DIQMTQSPSSLSASVGDRVTITCRASQGIRNDLGWYQQKPGKAPKRLIYAATSLQSGVPSRFSGSGSGTEFTLTISSLQPEDFATYYCLQHNNYPRTFGGGTKVEIK

SEQ ID NO: 25 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 26 - protein, 359 aa, Mus sp.
MEPISVSIYTSDNYSEEVGSGDYDSNKEPCFRDENVHFNRIFLPTIYFIIFLTGIVGNGLVILVMGYQKKLRSMTDKYRLHLSVADLLFVITLPFWAVDAMADWYFGKFLCKAVHIIYTVNLYSSVLILAFISLDRYLAIVHATNSQRPRKLLAEKAVYVGVWIPALLLTIPDFIFADVSQGDISQGDDRYICDRLYPDSLWMVVFQFQHIMVGLILPGIVILSCYCIIISKLSHSKGHQKRKALKTTVILILAFFACWLPYYVGISIDSFILLGVIKQGCDFESIVHKWISITEALAFFHCCLNPILYAFLGAKFKSSAQHALNSMSRGSSLKILSKGKRGGHSSVSTESESSSFHSS

SEQ ID NO: 27 - protein, 459 aa, Artificial Sequence
  Description of Artificial Sequence Synthetic polypeptide
QVQLVESGGGVVQPGRSLRLSCAASGFTFSNYVMHWVRQAPGKGLEWVAVIWYDGSNKYYADSVKGRFTISRDNSKNTLSLQMNSLRAEDTAVYYCERGEGYYGSGSRYRGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 214 aa, Artificial Sequence
  Description of Artificial Sequence Synthetic polypeptide
DIQMTQSPSSLSASVGDRVTITCRASQGIRTDLGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 122 aa, Artificial Sequence
  Description of Artificial Sequence Synthetic polypeptide
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARGIAAYYYYYGMDVWGQGTTVTVSS

SEQ ID NO: 30 - protein, 124 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARYYGSGSYYYYYGMDVWGQGTTVTVSS

SEQ ID NO: 31 - protein, 120 aa, Artificial Sequence
  Description of Artificial Sequence Synthetic polypeptide
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDCYSYYYGMDVWGQGTTVTVSS

SEQ ID NO: 32 - protein, 118 aa, Artificial Sequence
  Description of Artificial Sequence Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRNYYYGMDVWGQGTTVTVSS

SEQ ID NO: 33 - protein, 118 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARLRYFDWLDYWGQGTLVTVSS

SEQ ID NO: 34 - protein, 107 aa, Artificial Sequence
  Description of Artificial Sequence Synthetic polypeptide
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPLTFGGGTKVEIK

SEQ ID NO: 35 - protein, 107 aa, Artificial Sequence
  Description of Artificial Sequence Synthetic polypeptide
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIK

SEQ ID NO: 36 - protein, 111 aa, Artificial Sequence
  Description of Artificial Sequence Synthetic polypeptide
QSALTQPASVSGSPGQSITISCTGTSSDVGSYNLVSWYQQHPGKAPKLMIYEVSKRPSGVSNRFSGSKSGNTASLTISGLQAEDEADYYCCSYAGSSTFVVFGGGTKLTVL

SEQ ID NO: 37 - protein, 112 aa, Artificial Sequence
  Description of Artificial Sequence Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIK

SEQ ID NO: 38 - protein, 107 aa, Artificial Sequence
  Description of Artificial Sequence Synthetic polypeptide
DIQMTQSPSSLSASVGDRVTITCRASQDIRNDLGWYQQKPGKAPKRLIYAASSLQSGVPSRFSGSGSGTEFTLTISSLQPEDFATYYCLQHNSYPLTFGGGTKVQIK

SEQ ID NO: 39 - protein, 107 aa, Artificial Sequence
  Description of Artificial Sequence Synthetic polypeptide
DIQMTQSPSSLSASVGDRVTITCRASQDIRNDLGWYQQKPGKAPKRLIYAASSLQSGVPSRFSGSGSGTQFTLTISSLQPEDFATYYCLQHNSYPLTFGGGTKVEIK

SEQ ID NO: 40 - protein, 107 aa, Artificial Sequence
  Description of Artificial Sequence Synthetic polypeptide
DIQMTQSPSSLSASVGDRVTITCRASQDIRNDLGWYQQKPGKAPKRLIYAASSLQSGVPSRFSGSGSGTEFTLTISSLQPEDFATYYCLQHNSYPLTFGGGTKVEIK

SEQ ID NO: 41 - protein, 129 aa, Artificial Sequence
  Description of Artificial Sequence Synthetic polypeptide
QVQLVESGGGVVQPGRSLRLSCAASGFTFSSYVMHWVRQAPGKGLEWVAVIWYDGSNKYYADSVKGRFTISRDNSKNTLSLQMNSLRAEDTAVYYCERGEGYYGSGSRYRGYYYGMDVWGQGTTVTVSS

SEQ ID NO: 42 - protein, 129 aa, Artificial Sequence
  Description of Artificial Sequence Synthetic polypeptide
QVQLVESGGGVVQPGRSLRLSCAASGFTFSNYGMHWVRQAPGKGLEWVAVIWYDGSNKYYADSVKGRFTISRDNSKNTLSLQMNSLRAEDTAVYYCERGEGYYGSGSRYRGYYYGMDVWGQGTTVTVSS

SEQ ID NO: 43 - protein, 129 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLSLQMNSLRAEDTAVYYCERGEGYYGSGSRYRGYYYGMDVWGQGTTVTVSS

SEQ ID NO: 44 - protein, 129 aa, Artificial Sequence
  Description of Artificial Sequence Synthetic polypeptide
QVQLVESGGGVVQPGRSLRLSCAASGFTFSNYVMHWVRQAPGKGLEWVAVIWYDGSNKYYADSVKGRFTISRDNSKNTLYLQMNSLRAEDTAVYYCERGEGYYGSGSRYRGYYYGMDVWGQGTTVTVSS

SEQ ID NO: 45 - protein, 129 aa, Artificial Sequence
  Description of Artificial Sequence Synthetic polypeptide
QVQLVESGGGVVQPGRSLRLSCAASGFTFSSYVMHWVRQAPGKGLEWVAVIWYDGSNKYYADSVKGRFTISRDNSKNTLYLQMNSLRAEDTAVYYCERGEGYYGSGSRYRGYYYGMDVWGQGTTVTVSS

SEQ ID NO: 46 - protein, 129 aa, Artificial Sequence
  Description of Artificial Sequence Synthetic polypeptide
QVQLVESGGGVVQPGRSLRLSCAASGFTFSNYGMHWVRQAPGKGLEWVAVIWYDGSNKYYADSVKGRFTISRDNSKNTLYLQMNSLRAEDTAVYYCERGEGYYGSGSRYRGYYYGMDVWGQGTTVTVSS

SEQ ID NO: 47 - protein, 129 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCERGEGYYGSGSRYRGYYYGMDVWGQGTTVTVSS

SEQ ID NO: 48 - protein, 129 aa, Artificial Sequence
  Description of Artificial Sequence Synthetic polypeptide
QVQLVESGGGVVQPGRSLRLSCAASGFTFSNYVMHWVRQAPGKGLEWVAVIWYDGSNKYYADSVKGRFTISRDNSKNTLSLQMNSLRAEDTAVYYCARGEGYYGSGSRYRGYYYGMDVWGQGTTVTVSS

SEQ ID NO: 49 - protein, 129 aa, Artificial Sequence
  Description of Artificial Sequence Synthetic polypeptide
QVQLVESGGGVVQPGRSLRLSCAASGFTFSSYVMHWVRQAPGKGLEWVAVIWYDGSNKYYADSVKGRFTISRDNSKNTLSLQMNSLRAEDTAVYYCARGEGYYGSGSRYRGYYYGMDVWGQGTTVTVSS

SEQ ID NO: 50 - protein, 129 aa, Artificial Sequence
  Description of Artificial Sequence Synthetic polypeptide
QVQLVESGGGVVQPGRSLRLSCAASGFTFSNYGMHWVRQAPGKGLEWVAVIWYDGSNKYYADSVKGRFTISRDNSKNTLSLQMNSLRAEDTAVYYCARGEGYYGSGSRYRGYYYGMDVWGQGTTVTVSS

SEQ ID NO: 51 - protein, 129 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLSLQMNSLRAEDTAVYYCARGEGYYGSGSRYRGYYYGMDVWGQGTTVTVSS

SEQ ID NO: 52 - protein, 129 aa, Artificial Sequence
  Description of Artificial Sequence Synthetic polypeptide
QVQLVESGGGVVQPGRSLRLSCAASGFTFSNYVMHWVRQAPGKGLEWVAVIWYDGSNKYYADSVKGRFTISRDNSKNTLYLQMNSLRAEDTAVYYCARGEGYYGSGSRYRGYYYGMDVWGQGTTVTVSS

SEQ ID NO: 53 - protein, 129 aa, Artificial Sequence
  Description of Artificial Sequence Synthetic polypeptide
QVQLVESGGGVVQPGRSLRLSCAASGFTFSSYVMHWVRQAPGKGLEWVAVIWYDGSNKYYADSVKGRFTISRDNSKNTLYLQMNSLRAEDTAVYYCARGEGYYGSGSRYRGYYYGMDVWGQGTTVTVSS

SEQ ID NO: 54 - protein, 129 aa, Artificial Sequence
  Description of Artificial Sequence Synthetic polypeptide
QVQLVESGGGVVQPGRSLRLSCAASGFTFSNYGMHWVRQAPGKGLEWVAVIWYDGSNKYYADSVKGRFTISRDNSKNTLYLQMNSLRAEDTAVYYCARGEGYYGSGSRYRGYYYGMDVWGQGTTVTVSS

SEQ ID NO: 55 - protein, 129 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARGEGYYGSGSRYRGYYYGMDVWGQGTTVTVSS

SEQ ID NO: 56 - protein, 107 aa, Artificial Sequence
  Description of Artificial Sequence Synthetic polypeptide
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPRTFGQGTKVEIK

SEQ ID NO: 57 - protein, 107 aa, Artificial Sequence
  Description of Artificial Sequence Synthetic polypeptide
DIQMTQSPSSLSASVGDRVTITCRASQGIRTDLGWYQQKPGKAPKRLIYAASSLQSGVPSRFSGSGSGTEFTLTISSLQPEDFATYYCLQHNSYPWTFGQGTKVEIK

SEQ ID NO: 58 - protein, 107 aa, Artificial Sequence
  Description of Artificial Sequence Synthetic polypeptide
DIQMTQSPSSLSASVGDRVTITCRASQDIRNDLGWYQQKPGKAPTRLIYAASSLQSGVPSRFSGSGSGTEFTLTISSLQPEDFATYYCLQHNSYPLTFGGGTKVQIK

SEQ ID NO: 59 - protein, 107 aa, Artificial Sequence
  Description of Artificial Sequence Synthetic polypeptide
DIQMTQSPSSLSASVGDRVTITCRASQDIRNDLGWYQQKPGKAPTRLIYAASSLQSGVPSRFSGSGSGTQFTLTISSLQPEDFATYYCLQHNSYPLTFGGGTKVEIK

SEQ ID NO: 60 - protein, 107 aa, Artificial Sequence
  Description of Artificial Sequence Synthetic polypeptide
DIQMTQSPSSLSASVGDRVTITCRASQDIRNDLGWYQQKPGKAPTRLIYAASSLQSGVPSRFSGSGSGTEFTLTISSLQPEDFATYYCLQHNSYPLTFGGGTKVEIK